SEQUENCE LISTING (165 sequences)

SEQ ID NO: 1 - protein, 116 aa, Artificial Sequence
  anti-VEGF dAb DOM15-26-593
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 2 - protein, 94 aa, Artificial Sequence
  Anti-TNF-alpha adnectin
VSDVPRDLEVVAATPTSLLISWDTHNAYNGYYRITYGETGGNSPVREFTVPHPEVTATISGLKPGVDDTITVYAVTNHHMPLRIFGPISINHRT

SEQ ID NO: 3 - protein, 5 aa, Artificial Sequence
  G4S Linker
GGGGS

SEQ ID NO: 4 - protein, 6 aa, Artificial Sequence
  Linker
TVAAPS

SEQ ID NO: 5 - protein, 7 aa, Artificial Sequence
  Linker
ASTKGPT

SEQ ID NO: 6 - protein, 7 aa, Artificial Sequence
  Linker
ASTKGPS

SEQ ID NO: 7 - protein, 2 aa, Artificial Sequence
  Linker
GS

SEQ ID NO: 8 - protein, 8 aa, Artificial Sequence
  Linker
TVAAPSGS

SEQ ID NO: 9 - protein, 19 aa, Artificial Sequence
  Signal peptide sequence
MGWSCIILFLVATATGVHS

SEQ ID NO: 10 - protein, 445 aa, Homo sapiens
  Anti-TNF-alpha mAb (adalimumab) Heavy Chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 11 - DNA, 642 bp, Homo sapiens
  Anti-TNF-alpha mAb (adalimumab) Light Chain
gatatccagatgacccagagccccagcagcctgagcgcctctgtgggcgatagagtgaccatcacctgccgggccagccagggcatcagaaactacctggcctggtatcagcagaagcctggcaaggcccctaagctgctgatctacgccgccagcaccctgcagagcggcgtgcccagcagattcagcggcagcggctccggcaccgacttcaccctgaccatcagcagcctgcagcccgaggacgtggccacctactactgccagcggtacaacagagccccttacaccttcggccagggcaccaaggtggagatcaagcgtacggtggccgcccccagcgtgttcatcttcccccccagcgatgagcagctcaagagcggcaccgccagcgtggtgtgtctgctgaacaacttctacccccgggaggccaaagtgcagtggaaagtggacaacgccctgcagagcggcaacagccaggagagcgtgaccgagcaggacagcaaggactccacctacagcctgagcagcaccctgaccctgagcaaggccgactacgagaagcacaaagtgtacgcctgcgaagtgacccaccagggcctgtccagccccgtgaccaagagcttcaaccggggcgagtgc

SEQ ID NO: 12 - protein, 214 aa, Homo sapiens
  Anti-TNF-alpha mAb (adalimumab) Light Chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1710 bp, Artificial Sequence
  Anti-TNF-alpha mAb (adalimumab)-DOM15-26-593 Heavy Chain (DMS4000 mAbdAb heavy chain)
gaggtgcagctggtggagtctggcggcggactggtgcagcccggcagaagcctgagactgagctgtgccgccagcggcttcaccttcgacgactacgccatgcactgggtgaggcaggcccctggcaagggcctggagtgggtgtccgccatcacctggaatagcggccacatcgactacgccgacagcgtggagggcagattcaccatcagccgggacaacgccaagaacagcctgtacctgcagatgaacagcctgagagccgaggacaccgccgtgtactactgtgccaaggtgtcctacctgagcaccgccagcagcctggactactggggccagggcaccctggtgacagtctcgagcgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggagcccaagagctgcgataagacccacacctgccccccctgccctgcccccgagctgctgggcggacctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagcctagagagccccaggtctacaccctgcctccctccagagatgagctgaccaagaaccaggtgtccctgacctgtctggtgaagggcttctaccccagcgacatcgccgtggagtgggagagcaacggccagcccgagaacaactacaagaccaccccccctgtgctggacagcgatggcagcttcttcctgtactccaagctgaccgtggacaagagcagatggcagcagggcaacgtgttcagctgcagcgtgatgcacgaggccctgcacaatcactacacccagaagagtctgagcctgtcccctggcaagtcgaccggtgaggtgcagctgttggtgtctgggggaggcttggtacagcctggggggtccctgcgtctctcctgtgcagcctccggattcacctttaaggcttatccgatgatgtgggtccgccaggctccagggaagggtctagagtgggtttcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagttagactactggggtcagggaaccctggtcaccgtctcgagc

SEQ ID NO: 14 - protein, 564 aa, Artificial Sequence
  Anti-TNF-alpha mAb (adalimumab)-DOM15-26-593 Heavy Chain (DMS4000 mAbdAb heavy chain)
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKSTGEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 15 - protein, 568 aa, Artificial Sequence
  DOM 15-26-anti-TNF-alpha mAb (adalimumab) Heavy Chain
EVQLLESGGGLVQPGGSLRLSCAASGFTFGAYPMMWVRQAPGKGLEWVSEISPSGSYTYYADSVKGRFTISRDNSKNTLYLQMNSLRAEDTAVYYCAKDPRKFDYWGQGTLVTVSSASTKGPSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 16 - protein, 556 aa, Artificial Sequence
  Anti-TNF-alpha mAb (adalimumab)-DOM15-10-11 Heavy Chain (DMS4031 mAbdAb heavy chain)
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKSTGDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 17 - protein, 119 aa, Artificial Sequence
  Anti-TNFR1 dAb (DOM1h-131-206)
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSS

SEQ ID NO: 18 - protein, 86 aa, Artificial Sequence
  Anti-VEGFR2 adnectin
EVVAATPTSLLISWRHPHFPTRYYRITYGETGGNSPVQEFTVPLQPPTATISGLKPGVDYTITVYAVTDGRNGRLLSIPISINYRT

SEQ ID NO: 19 - protein, 150 aa, Artificial Sequence
  Anti-VEGF anticalin
DGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 20 - protein, 447 aa, Artificial Sequence
  Alternative Anti-VEGF antibody Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 21 - protein, 214 aa, Artificial Sequence
  Anti-VEGF antibody (bevacizumab) Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 447 aa, Artificial Sequence
  Alternative Anti-VEGF antibody (bevacizumab) Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 23 - protein, 116 aa, Artificial Sequence
  anti-VEGF dAb DOM15-26
EVQLLESGGGLVQPGGSLRLSCAASGFTFGAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 24 - protein, 568 aa, Artificial Sequence
  DOM15-26-593-Anti-TNF-alpha mAb (adalimumab) Heavy Chain
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTKGPSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 25 - protein, 3 aa, Artificial Sequence
  Linker
STG

SEQ ID NO: 26 - DNA, 1335 bp, Artificial Sequence
  BPC1821 (CTLA4-Ig fused to anti-VEGFR2 adnectin via a GS linker)
atgcatgtcgcccagccagcggtggtgctggccagctcccgcggcattgcctccttcgtgtgcgagtacgccagccccggcaaggccaccgaggtgcgcgtcacggtgctccgccaggccgatagccaggtgaccgaagtgtgtgccgctacgtacatgatggggaacgagctgaccttcctggacgactctatctgcaccgggacctcgagcgggaaccaggtgaacctgaccatccagggcctgcgcgcgatggacacgggcctgtacatctgcaaggtggagttgatgtacccccccccgtactacctggggatcggcaacggcacgcagatctacgtcatcgaccccgaaccttgccctgacagcgaccaggagcccaagtctagtgacaagacccatacctctccccccagccccgctccagagctgctggggggctccagcgtgttcctgtttccccccaagcctaaggacaccctgatgatctccagaacccccgaggtgacctgcgtggtcgtggatgtgagtcacgaggaccctgaggtgaagttcaactggtacgtggacggggtggaggtgcataacgccaagaccaagcctcgcgaggagcagtacaacagtacctaccgcgtggtgtccgtgctcactgtgctgcatcaggactggctgaacggcaaggagtataagtgcaaggtgtctaacaaggccttgcccgcccccatcgagaaaacaatctccaaggccaaagggcagcccagggaacctcaggtgtacaccctccctccaagccgtgacgagctgaccaagaaccaggtctctctgacctgcttggtgaagggcttctaccctagcgacatcgctgtggagtgggagtccaacgggcagcccgagaacaactacaaaaccaccccgcccgtgctggactctgacggctccttcttcctgtacagcaaactgaccgtggacaagtccaggtggcagcagggaaacgtgttcagctgcagcgtcatgcatgaggccctgcataaccattacacacagaagagcctgtccctgagccccggcaagggatccgaggtggtggccgccacccccaccagcctgctgatttcctggaggcacccccacttccccacacgctactacaggatcacctacggcgagaccggcggcaacagccccgtgcaggagttcaccgtgcccctgcagcctcccactgccaccatcagcggcctcaagcccggcgtggactacaccatcaccgtgtacgccgtcaccgacggaaggaacggcaggctgctgagcatccccatcagcatcaactacaggacc

SEQ ID NO: 27 - protein, 437 aa, Artificial Sequence
  BPC1821 (CTLA4-Ig fused to anti-VEGFR2 adnectin via a GS linker)
MHVAQPAVVLASSRGIASFVCEYDGKATEVRVTVLRQADSQVTEVCAATYMMGNELTFLDDSICTGTSSGNQVNLTIQGLRAMDTGICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKGSEVVAATPTSLLISWRHPHFPTRYYRITYGETGGNSPVQEFTVPLQPPTATISGLKPGVDYTITVYAVTDGRNGRLLSIPISINYRT

SEQ ID NO: 28 - DNA, 1425 bp, Artificial Sequence
  BPC1825 (CTLA4-Ig fused to an anti-VEGF dAb via a GS linker)
atgcatgtcgcccagccagcggtggtgctggccagctcccgcggcattgcctccttcgtgtgcgagtacgccagccccggcaaggccaccgaggtgcgcgtcacggtgctccgccaggccgatagccaggtgaccgaagtgtgtgccgctacgtacatgatggggaacgagctgaccttcctggacgactctatctgcaccgggacctcgagcgggaaccaggtgaacctgaccatccagggcctgcgcgcgatggacacgggcctgtacatctgcaaggtggagttgatgtacccccccccgtactacctggggatcggcaacggcacgcagatctacgtcatcgaccccgaaccttgccctgacagcgaccaggagcccaagtctagtgacaagacccatacctctccccccagccccgctccagagctgctggggggctccagcgtgttcctgtttccccccaagcctaaggacaccctgatgatctccagaacccccgaggtgacctgcgtggtcgtggatgtgagtcacgaggaccctgaggtgaagttcaactggtacgtggacggggtggaggtgcataacgccaagaccaagcctcgcgaggagcagtacaacagtacctaccgcgtggtgtccgtgctcactgtgctgcatcaggactggctgaacggcaaggagtataagtgcaaggtgtctaacaaggccttgcccgcccccatcgagaaaacaatctccaaggccaaagggcagcccagggaacctcaggtgtacaccctccctccaagccgtgacgagctgaccaagaaccaggtctctctgacctgcttggtgaagggcttctaccctagcgacatcgctgtggagtgggagtccaacgggcagcccgagaacaactacaaaaccaccccgcccgtgctggactctgacggctccttcttcctgtacagcaaactgaccgtggacaagtccaggtggcagcagggaaacgtgttcagctgcagcgtcatgcatgaggccctgcataaccattacacacagaagagcctgtccctgagccccggcaagggatccgaggtgcagctcctggtcagcggcggcggcctggtccagcccggaggctcactgaggctgagctgcgccgctagcggcttcaccttcaaggcctaccccatgatgtgggtcaggcaggcccccggcaaaggcctggagtgggtgtctgagatcagccccagcggcagctacacctactacgccgacagcgtgaagggcaggttcaccatcagcagggacaacagcaagaacaccctgtacctgcagatgaactctctgagggccgaggacaccgccgtgtactactgcgccaaggaccccaggaagctggactattggggccagggcactctggtgaccgtgagcagc

SEQ ID NO: 29 - protein, 467 aa, Artificial Sequence
  BPC1825 (CTLA4-Ig fused to an anti-VEGF dAb via a GS linker)
MHVAQPAVVLASSRGIASFVCEYDGKATEVRVTVLRQADSQVTEVCAATYMMGNELTFLDDSICTGTSSGNQVNLTIQGLRAMDTGICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKGSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 30 - protein, 126 aa, Artificial Sequence
  Anti-TNF-alpha mAb heavy chain
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 31 - protein, 107 aa, Artificial Sequence
  Anti-TNF-alpha mAb light chain
EIVLTQSPATLSLSPGERATLSCRASQSVYSYLAWYQQKPGQAPRLLIYDNRATGIPARFSGSGSGTDFTLTISSLEPEDFAVYYCQQRSNWPPFTFGPGTKVDIKR

SEQ ID NO: 32 - protein, 444 aa, Artificial Sequence
  Anti-TNF-alpha mAb (Infliximab) Heavy chain
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 33 - protein, 214 aa, Artificial Sequence
  Anti-TNF-alpha mAb (Infliximab) Light chain
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 463 aa, Artificial Sequence
  TNFR-Fc fusion (Etanercept)
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 35 - protein, 108 aa, Artificial Sequence
  Anti-TNF-alpha Vk dAb (PEP1-5-19)
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 36 - protein, 106 aa, Artificial Sequence
  Anti-TNF-alpha Vk dAb (PEP1-5-490)
DIQMTQSPSSLSASVGDRVTITCRASQSIDSYLHWYQQKPGKAPKLLIYSNLETGVPSRFSGSGSGTDFTLTISSLQPEDFATYYCQQVVWRPFTFGQGTKVEIKR

SEQ ID NO: 37 - protein, 107 aa, Artificial Sequence
  Anti-TNF-alpha Vk dAb (PEP1-5-493)
DIQMTQSPSSLSASVGDRVTITCRASQAIDSYLHWYQQKPGKAPKLLIYSNLETGVPSRFSGSGSGTDFTLTISSLLIPEDFATYYCQQVVWRPFTFGQGTKVEIKR

SEQ ID NO: 38 - protein, 245 aa, Artificial Sequence
  Anti-TNF-alpha scFv (ESBA105)
DIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSS

SEQ ID NO: 39 - protein, 229 aa, Artificial Sequence
  Anti-VEGF Fab (ranibizumab) Heavy Chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHL

SEQ ID NO: 40 - protein, 214 aa, Artificial Sequence
  Anti-VEGF Fab (ranibizumab) Light Chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 126 aa, Artificial Sequence
  Anti-VEGF antibody (R84) Heavy chain
QVQLVQSGAEVKKPGASVKVSCKASGGTFSSYAISWVRQAPGQGLEWMGGFDPEDGETIYAQKFQGRVTMTEDTSTDTAYMELSSLRSEDTAVYYCATGRSMFRGVIIPFNGMDVWGQGTTVTVSS

SEQ ID NO: 42 - protein, 108 aa, Artificial Sequence
  Anti-VEGF antibody (R84) light chain
DIRMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 43 - protein, 424 aa, Artificial Sequence
  VEGFR1/2 hybrid - Fc fusion (aflibercept - VEGF-Trap)
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTQTNTIIDVVLSPSHGIELSVGEKLVLNCTARTELNVGIDFNWEYPSSKHQHKKLVNRDLKTQSGSEMKKFLSTLTIDGVTRSDQGTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 44 - protein, 108 aa, Artificial Sequence
  Anti-VEGF Vk dAb (DOM15-10-11)
DIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIRG

SEQ ID NO: 45 - protein, 86 aa, Artificial Sequence; identical to SEQ ID NO: 18
  CT01

SEQ ID NO: 46 - DNA, 1767 bp, Artificial Sequence
  Anti-TNF-alpha mAb (adalimumab)-DOM15-26-593 Heavy Chain FC disabled (DMS4000 mAbdAb heavy chain Fc disabled)
atgggctggtcctgcatcatcctgtttctggtggccaccgccaccggcgtgcacagcgaggtgcagctggtggagtctggcggcggactggtgcagcccggcagaagcctgagactgagctgtgccgccagcggcttcaccttcgacgactacgccatgcactgggtgaggcaggcccctggcaagggcctggagtgggtgtccgccatcacctggaatagcggccacatcgactacgccgacagcgtggagggcagattcaccatcagccgggacaacgccaagaacagcctgtacctgcagatgaacagcctgagagccgaggacaccgccgtgtactactgtgccaaggtgtcctacctgagcaccgccagcagcctggactactggggccagggcaccctggtgacagtctcgagcgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggagcccaagagctgcgataagacccacacctgccccccctgccctgcccccgagctggccggcgcccctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagcctagagagccccaggtctacaccctgcctccctccagagatgagctgaccaagaaccaggtgtccctgacctgtctggtgaagggcttctaccccagcgacatcgccgtggagtgggagagcaacggccagcccgagaacaactacaagaccaccccccctgtgctggacagcgatggcagcttcttcctgtactccaagctgaccgtggacaagagcagatggcagcagggcaacgtgttcagctgcagcgtgatgcacgaggccctgcacaatcactacacccagaagagtctgagcctgtcccctggcaagtcgaccggtgaggtgcagctgctggtgtctggcggcggactggtgcagcctggcggcagcctgagactgagctgcgccgccagcggcttcaccttcaaggcctaccccatgatgtgggtgcggcaggcccctggcaagggcctggaatgggtgtccgagatcagccccagcggcagctacacctactacgccgacagcgtgaagggccggttcaccatcagccgggacaacagcaagaacaccctgtacctgcagatgaacagcctgcgggccgaggacaccgccgtgtactactgcgccaaggacccccggaagctggactactggggccagggcaccctggtgaccgtgagcagc

SEQ ID NO: 47 - protein, 564 aa, Artificial Sequence
  Anti-TNF-alpha mAb (adalimumab)-DOM15-26-593 Heavy Chain FC disabled (DMS4000 mAbdAb heavy chain Fc disabled)
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKSTGEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 48 - DNA, 1758 bp, Artificial Sequence
  EtanSTG593
ctgcccgctcaggtggccttcactccctacgccccagagcccggctctacctgcaggctgagggagtactacgaccagaccgcccagatgtgctgcagcaagtgcagccccggccagcacgccaaagtgttctgcaccaagaccagcgacaccgtgtgcgatagctgcgaggacagcacctacacccagctgtggaactgggtccccgagtgcctgagctgcggctctaggtgtagcagcgaccaggtcgagacccaggcctgcaccagggaacagaaccggatctgcacatgcaggcccggctggtactgcgccctcagcaaacaggagggctgcaggctgtgtgcccccctcaggaagtgcaggcccgggtttggcgtggccaggcccggaaccgagactagcgacgtggtgtgcaaaccctgcgcccccggcaccttcagcaataccactagcagcaccgacatctgcaggcctcaccagatctgcaacgtggtggccattcccggcaacgcaagcatggacgccgtgtgcaccagcaccagccccaccaggtcaatggcccctggagccgtgcatctgccccagcccgtgagcaccagaagccagcacacccagcctacccccgagcccagcaccgcccctagcaccagcttcctgctgcctatgggcccctcccctcccgccgagggctcaaccggcgacgaacccaagagctgcgacaagacccacacctgccccccctgccccgcaccagaactcctgggcggacccagcgtgttcctgttcccccccaagcccaaggacaccctgatgatcagcaggacccccgaggtgacctgtgtggtggtggacgtgagccacgaggaccccgaggtgaagttcaactggtacgtggacggcgtggaggtgcacaacgccaagaccaagcccagggaggagcagtacaacagcacctacagggtggtgagcgtcctgaccgtgctgcaccaggactggctgaacggcaaggagtacaagtgcaaggtgagcaacaaggccctgcccgcccccatcgagaagaccatcagcaaggccaaaggccagcccagggagccacaggtgtacacactgccccccagcagggaggagatgaccaagaaccaggtgagcctgacctgcctggtgaagggcttctatcccagcgatatcgccgtggagtgggagagcaacggccagcccgagaacaactacaagaccaccccccccgtcctggactccgacgggagcttcttcctgtacagcaagctgaccgtggacaagagcaggtggcagcagggcaacgtgttcagctgcagcgtgatgcacgaggccctgcacaaccactacacccagaagtccctgagcctgagccccggcaagtcgaccggtgaggtgcagctgctggtgtctggcggcggactggtgcagcctggcggcagcctgagactgagctgcgccgccagcggcttcaccttcaaggcctaccccatgatgtgggtgcggcaggcccctggcaagggcctggaatgggtgtccgagatcagccccagcggcagctacacctactacgccgacagcgtgaagggccggttcaccatcagccgggacaacagcaagaacaccctgtacctgcagatgaacagcctgcgggccgaggacaccgccgtgtactactgcgccaaggacccccggaagctggactactggggccagggcaccctggtgaccgtgagcagc

SEQ ID NO: 49 - protein, 582 aa, Artificial Sequence
  EtanSTG593
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGKSTGEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 50 - DNA, 1827 bp, Artificial Sequence
  EtanTV4593
ctgcccgctcaggtggccttcactccctacgccccagagcccggctctacctgcaggctgagggagtactacgaccagaccgcccagatgtgctgcagcaagtgcagccccggccagcacgccaaagtgttctgcaccaagaccagcgacaccgtgtgcgatagctgcgaggacagcacctacacccagctgtggaactgggtccccgagtgcctgagctgcggctctaggtgtagcagcgaccaggtcgagacccaggcctgcaccagggaacagaaccggatctgcacatgcaggcccggctggtactgcgccctcagcaaacaggagggctgcaggctgtgtgcccccctcaggaagtgcaggcccgggtttggcgtggccaggcccggaaccgagactagcgacgtggtgtgcaaaccctgcgcccccggcaccttcagcaataccactagcagcaccgacatctgcaggcctcaccagatctgcaacgtggtggccattcccggcaacgcaagcatggacgccgtgtgcaccagcaccagccccaccaggtcaatggcccctggagccgtgcatctgccccagcccgtgagcaccagaagccagcacacccagcctacccccgagcccagcaccgcccctagcaccagcttcctgctgcctatgggcccctcccctcccgccgagggctcaaccggcgacgaacccaagagctgcgacaagacccacacctgccccccctgccccgcaccagaactcctgggcggacccagcgtgttcctgttcccccccaagcccaaggacaccctgatgatcagcaggacccccgaggtgacctgtgtggtggtggacgtgagccacgaggaccccgaggtgaagttcaactggtacgtggacggcgtggaggtgcacaacgccaagaccaagcccagggaggagcagtacaacagcacctacagggtggtgagcgtcctgaccgtgctgcaccaggactggctgaacggcaaggagtacaagtgcaaggtgagcaacaaggccctgcccgcccccatcgagaagaccatcagcaaggccaaaggccagcccagggagccacaggtgtacacactgccccccagcagggaggagatgaccaagaaccaggtgagcctgacctgcctggtgaagggcttctatcccagcgatatcgccgtggagtgggagagcaacggccagcccgagaacaactacaagaccaccccccccgtcctggactccgacgggagcttcttcctgtacagcaagctgaccgtggacaagagcaggtggcagcagggcaacgtgttcagctgcagcgtgatgcacgaggccctgcacaaccactacacccagaagtccctgagcctgagccccggcaagaccgtggcggcgcccagcacggtggccgccccctccaccgtcgccgcgccaagcaccgtggctgctccgtcgaccggtgaggtgcagctgctggtgtctggcggcggactggtgcagcctggcggcagcctgagactgagctgcgccgccagcggcttcaccttcaaggcctaccccatgatgtgggtgcggcaggcccctggcaagggcctggaatgggtgtccgagatcagccccagcggcagctacacctactacgccgacagcgtgaagggccggttcaccatcagccgggacaacagcaagaacaccctgtacctgcagatgaacagcctgcgggccgaggacaccgccgtgtactactgcgccaaggacccccggaagctggactactggggccagggcaccctggtgaccgtgagcagc

SEQ ID NO: 51 - protein, 605 aa, Artificial Sequence
  EtanTV4593
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSTVAAPSTVAAPSTVAAPSTGEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 52 - DNA, 37 bp, Artificial Sequence
  AVG18 primer
attatgggatccaccggcgaggtgcagctgttggtgt

SEQ ID NO: 53 - DNA, 40 bp, Artificial Sequence
  AVG19 primer
gctggggcccttggtgctagcgctcgagacggtgaccagg

SEQ ID NO: 54 - DNA, 44 bp, Artificial Sequence
  AVG26 primer
ctcgagcgctagcaccaagggccccagcgacatccagatgaccc

SEQ ID NO: 55 - DNA, 37 bp, Artificial Sequence
  AVG21 primer
ttatgtcaagcttttaccgtttgatttccaccttggt

SEQ ID NO: 56 - DNA, 41 bp, Artificial Sequence
  AVG22 primer
attatgggatccaccggcgacatccagatgacccagtctcc

SEQ ID NO: 57 - DNA, 37 bp, Artificial Sequence
  AVG36 primer
gcgccgccaccgtacgtttgatttccaccttggtccc

SEQ ID NO: 58 - DNA, 45 bp, Artificial Sequence
  AVG37 primer
caaacgtacggtggcggcgccgagcgaggtgcagctgttggtgtc

SEQ ID NO: 59 - DNA, 34 bp, Artificial Sequence
  AVG25 primer
ttatgtcaagcttttagctcgagacggtgaccag

SEQ ID NO: 60 - DNA, 36 bp, Artificial Sequence
  AVG24 primer
ggtggaaatcaaacgtacggtggcggcgccgagcga

SEQ ID NO: 61 - DNA, 693 bp, Artificial Sequence
  DOM15-26-593 - PEP1-5-19 in-line fusion
gaggtgcagctgttggtgtctgggggaggcttggtacagcctggggggtccctgcgtctctcctgtgcagcctccggattcacctttaaggcttatccgatgatgtgggtccgccaggctccagggaagggtctagagtgggtttcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagttagactactggggtcagggaaccctggtcaccgtctcgagcgctagcaccaagggccccagcgacatccagatgacccagtctccatcctctctgtctgcatctgtaggagaccgtgtcaccatcacttgccgggcaagtcagagcattgatagttatttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgg

SEQ ID NO: 62 - protein, 231 aa, Artificial Sequence
  DOM15-26-593 - PEP1-5-19 in-line fusion
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTKGPSDIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 63 - DNA, 690 bp, Artificial Sequence
  PEP1-5-19-DOM15-26-593 in-line fusion
gacatccagatgacccagtctccatcctctctgtctgcatctgtaggagaccgtgtcaccatcacttgccgggcaagtcagagcattgatagttatttacattggtaccagcagaaaccagggaaagcccctaagctcctgatctatagtgcatccgagttgcaaagtggggtcccatcacgtttcagtggcagtggatctgggacagatttcactctcaccatcagcagtctgcaacctgaagattttgctacgtactactgtcaacaggttgtgtggcgtccttttacgttcggccaagggaccaaggtggaaatcaaacgtacggtggcggcgccgagcgaggtgcagctgttggtgtctgggggaggcttggtacagcctggggggtccctgcgtctctcctgtgcagcctccggattcacctttaaggcttatccgatgatgtgggtccgccaggctccagggaagggtctagagtgggtttcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagttagactactggggtcagggaaccctggtcaccgtctcgagc

SEQ ID NO: 64 - protein, 230 aa, Artificial Sequence
  PEP1-5-19-DOM15-26-593 in-line fusion
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKRTVAAPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 65 - protein, 352 aa, Artificial Sequence
  DMS1571 - a myc tagged Fc formatted version of the DOM 15-26-593 anti-VEGF dAb (exists as a dimer of this sequence)
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKGSEQKLISEEDLN

SEQ ID NO: 66 - protein, 18 aa, Artificial Sequence
  Linker
GSTVAAPSGSTVAAPSGS

SEQ ID NO: 67 - protein, 26 aa, Artificial Sequence
  Linker
GSTVAAPSGSTVAAPSGSTVAAPSGS

SEQ ID NO: 68 - protein, 34 aa, Artificial Sequence
  Linker
GSTVAAPSGSTVAAPSGSTVAAPSGSTVAAPSGS

SEQ ID NO: 69 - protein, 571 aa, Artificial Sequence
  Anti-TNF-alpha mAb (adalimumab) Fc disabled-DOM15-26-593 Heavy Chain with GSTVAAPSGS linker
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKGSTVAAPSGSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 70 - protein, 579 aa, Artificial Sequence
  Anti-TNF-alpha mAb (adalimumab) Fc disabled -DOM15-26-593 Heavy Chain with GS(TVAAPSGS) x2 linker
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKGSTVAAPSGSTVAAPSGSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 71 - protein, 587 aa, Artificial Sequence
  Anti-TNF-alpha mAb (adalimumab) Fc disabled -DOM15-26-593 Heavy Chain with GS(TVAAPSGS) x3 linker
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKGSTVAAPSGSTVAAPSGSTVAAPSGSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 72 - protein, 595 aa, Artificial Sequence
  Anti-TNF-alpha mAb (adalimumab) Fc disabled -DOM15-26-593 Heavy Chain with GS(TVAAPSGS) x4 linker
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKGSTVAAPSGSTVAAPSGSTVAAPSGSTVAAPSGSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 73 - protein, 585 aa, Artificial Sequence
  Etanercept-DOM15-26-593
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 74 - protein, 577 aa, Artificial Sequence [sequence omitted]

SEQ ID NO: 75 - protein, 619 aa, Artificial Sequence [sequence omitted]

SEQ ID NO: 76 - protein, 554 aa, Artificial Sequence
  Infliximab-bevacizumab DVD-Ig heavy chain
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSV

SEQ ID NO: 77 - protein, 328 aa, Artificial Sequence
  Infliximab-bevacizumab DVD-Ig light chain
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 577 aa, Artificial Sequence
  Infliximab-r84 DVD-Ig heavy chain
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSQVQLVQSGAEVKKPGASVKVSCKASGGTFSSYAISWVRQAPGQGLEWMGGFDPEDGETIYAQKFQGRVTMTEDTSTDTAYMELSSLRSEDTAVYYCATGRSMFRGVIIPFNGMDVWGQGTTVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 79 - protein, 328 aa, Artificial Sequence
  Infliximab-r84 DVD-Ig light chain
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSDIRMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 356 aa, Artificial Sequence [sequence omitted]

SEQ ID NO: 81 - protein, 328 aa, Artificial Sequence
  Infliximab-ranibizumab DVD-Fab
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 566 aa, Artificial Sequence
  Infliximab-DOM15-26-593 mAb-dAb heavy chain
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 83 - protein, 558 aa, Artificial Sequence
  Infliximab-DOM15-10-11 mAb-dAb heavy chain
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 84 - protein, 600 aa, Artificial Sequence [sequence omitted]

SEQ ID NO: 85 - protein, 336 aa, Artificial Sequence
  Infliximab-DOM15-26-593 mAb-dAb light chain
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 86 - protein, 328 aa, Artificial Sequence
  Infliximab-DOM15-10-11 mAb-dAb light chain
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 87 - protein, 370 aa, Artificial Sequence
  Infliximab-VEGF anticalin light chain
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSDGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 88 - protein, 575 aa, Artificial Sequence
  Adalimumab-bevacizumab DVD-Ig heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 89 - protein, 328 aa, Artificial Sequence
  Adalimumab-bevacizumab DVD-Ig light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 578 aa, Artificial Sequence
  Adalimumab-r84 DVD-Ig heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSQVQLVQSGAEVKKPGASVKVSCKASGGTFSSYAISWVRQAPGQGLEWMGGFDPEDGETIYAQKFQGRVTMTEDTSTDTAYMELSSLRSEDTAVYYCATGRSMFRGVIIPFNGMDVWGQGTTVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 91 - protein, 328 aa, Artificial Sequence
  Adalimumab-r84 DVD-Ig light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSDIRMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 357 aa, Artificial Sequence
  Adalimumab-ranibizumab DVD-Fab
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHL

SEQ ID NO: 93 - protein, 328 aa, Artificial Sequence
  Adalimumab-ranibizumab DVD-Fab
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 601 aa, Artificial Sequence
  Adalimumab-VEGF anticalin heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSDGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 95 - protein, 336 aa, Artificial Sequence
  Adalimumab-DOM15-26-593 mAb-dAb light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 96 - protein, 328 aa, Artificial Sequence
  Adalimumab-DOM15-10-11 mAb-dAb light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 97 - protein, 370 aa, Artificial Sequence
  Adalimumab-VEGF anticalin light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSDGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 98 - protein, 580 aa, Artificial Sequence
  anti- TNF-alpha mAb -bevacizumab DVD-Ig heavy chain
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 99 - protein, 327 aa, Artificial Sequence
  anti- TNF-alpha mAb -bevacizumab DVD-Ig light chain
EIVLTQSPATLSLSPGERATLSCRASQSVYSYLAWYQQKPGQAPRLLIYDNRATGIPARFSGSGSGTDFTLTISSLEPEDFAVYYCQQRSNWPPFTFGPGTKVDIKRTVAAPSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 583 aa, Artificial Sequence [sequence omitted]

SEQ ID NO: 101 - protein, 327 aa, Artificial Sequence
  anti- TNF-alpha mAb -r84 DVD-Ig light chain
EIVLTQSPATLSLSPGERATLSCRASQSVYSYLAWYQQKPGQAPRLLIYDNRATGIPARFSGSGSGTDFTLTISSLEPEDFAVYYCQQRSNWPPFTFGPGTKVDIKRTVAAPSDIRMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 362 aa, Artificial Sequence
  anti- TNF-alpha mAb -ranibizumab DVD-Fab heavy chain
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHL

SEQ ID NO: 103 - protein, 327 aa, Artificial Sequence
  anti- TNF-alpha mAb -ranibizumab DVD-Fab light chain
EIVLTQSPATLSLSPGERATLSCRASQSVYSYLAWYQQKPGQAPRLLIYDNRATGIPARFSGSGSGTDFTLTISSLEPEDFAVYYCQQRSNWPPFTFGPGTKVDIKRTVAAPSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 572 aa, Artificial Sequence
  anti- TNF-alpha mAb -DOM15-26-593 mAb-dAb heavy chain
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 105 - protein, 564 aa, Artificial Sequence
  anti- TNF-alpha mAb -DOM15-10-11 mAb-dAb heavy chain
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 106 - protein, 606 aa, Artificial Sequence
  anti- TNF-alpha mAb -VEGF anticalin heavy chain
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSDGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 107 - protein, 335 aa, Artificial Sequence
  anti- TNF-alpha mAb -DOM15-26-593 mAb-dAb light chain
EIVLTQSPATLSLSPGERATLSCRASQSVYSYLAWYQQKPGQAPRLLIYDNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 108 - protein, 327 aa, Artificial Sequence
  anti- TNF-alpha mAb -DOM15-10-11 mAb-dAb light chain
EIVLTQSPATLSLSPGERATLSCRASQSVYSYLAWYQQKPGQAPRLLIYDNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 109 - protein, 369 aa, Artificial Sequence
  anti- TNF-alpha mAb -VEGF anticalin light chain
EIVLTQSPATLSLSPGERATLSCRASQSVYSYLAWYQQKPGQAPRLLIYDNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSDGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 110 - protein, 571 aa, Artificial Sequence
  ESBA105-bevacizumab DVD-Ig heavy chain
QVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSSASTKGPSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 111 - protein, 328 aa, Artificial Sequence
  ESBA105-bevacizumab DVD-Ig light chain
DIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRTVAAPSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 574 aa, Artificial Sequence
  ESBA105-r84 DVD-Ig heavy chain
QVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSSASTKGPSQVQLVQSGAEVKKPGASVKVSCKASGGTFSSYAISWVRQAPGQGLEWMGGFDPEDGETIYAQKFQGRVTMTEDTSTDTAYMELSSLRSEDTAVYYCATGRSMFRGVIIPFNGMDVWGQGTTVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 113 - protein, 441 aa, Artificial Sequence [sequence omitted]

SEQ ID NO: 114 - protein, 353 aa, Artificial Sequence
  ESBA105-ranibizumab DVD-Fab heavy chain
QVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSSASTKGPSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHL

SEQ ID NO: 115 - protein, 328 aa, Artificial Sequence
  ESBA105-ranibizumab DVD-Fab light chain
DIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRTVAAPSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 368 aa, Artificial Sequence
  ESBA105-DOM15-26-593 scFv-VH dAb
DIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSSASTKGPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 117 - protein, 360 aa, Artificial Sequence
  ESBA105-DOM15-10-11 scFv-Vk dAb
DIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSSASTKGPSDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 118 - protein, 402 aa, Artificial Sequence
  ESBA105-VEGF anticalin
DIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSSASTKGPSDGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 119 - protein, 222 aa, Artificial Sequence
  PEP1-5-19-DOM15-10-11 dAb-dAb
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKRTVAAPSDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 120 - protein, 264 aa, Artificial Sequence
  PEP1-5-19-VEGF anticalin
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKRTVAAPSDGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 121 - protein, 216 aa, Artificial Sequence
  Anti-TNF adnectin-DOM15-26-593
VSDVPRDLEVVAATPTSLLISWDTHNAYNGYYRITYGETGGNSPVREFTVPHPEVTATISGLKPGVDDTITVYAVTNHHMPLRIFGPISINHRTTVAAPSEVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 122 - protein, 208 aa, Artificial Sequence
  Anti-TNF adnectin-DOM15-10-11
VSDVPRDLEVVAATPTSLLISWDTHNAYNGYYRITYGETGGNSPVREFTVPHPEVTATISGLKPGVDDTITVYAVTNHHMPLRIFGPISINHRTTVAAPSDIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKR

SEQ ID NO: 123 - protein, 250 aa, Artificial Sequence
  Anti-TNF adnectin-VEGF anticalin
VSDVPRDLEVVAATPTSLLISWDTHNAYNGYYRITYGETGGNSPVREFTVPHPEVTATISGLKPGVDDTITVYAVTNHHMPLRIFGPISINHRTTVAAPSDGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 124 - protein, 698 aa, Artificial Sequence
  Bevacizumab-ESBA105 mAb-scFv, heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSDIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSS

SEQ ID NO: 125 - protein, 465 aa, Artificial Sequence
  Bevacizumab-ESBA105 mAb-scFv, light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSDIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSS

SEQ ID NO: 126 - protein, 561 aa, Artificial Sequence
  Bevacizumab-PEP1-5-19 mAb-dAb heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSDIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 127 - protein, 328 aa, Artificial Sequence
  Bevacizumab-PEP1-5-19 mAb-dAb light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSDIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 128 - protein, 547 aa, Artificial Sequence
  Bevacizumab-TNF adnectin heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSVSDVPRDLEVVAATPTSLLISWDTHNAYNGYYRITYGETGGNSPVREFTVPHPEVTATISGLKPGVDDTITVYAVTNHHMPLRIFGPISINHRT

SEQ ID NO: 129 - protein, 314 aa, Artificial Sequence
  Bevacizumab-TNF adnectin light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSVSDVPRDLEVVAATPTSLLISWDTHNAYNGYYRITYGETGGNSPVREFTVPHPEVTATISGLKPGVDDTITVYAVTNHHMPLRIFGPISINHRT

SEQ ID NO: 130 - protein, 675 aa, Artificial Sequence [sequence omitted]

SEQ ID NO: 131 - protein, 538 aa, Artificial Sequence
  Aflibercept-PEP1-5-19
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTQTNTIIDVVLSPSHGIELSVGEKLVLNCTARTELNVGIDFNWEYPSSKHQHKKLVNRDLKTQSGSEMKKFLSTLTIDGVTRSDQGTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSDIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 132 - protein, 524 aa, Artificial Sequence
  Aflibercept-TNF adnectin
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTQTNTIIDVVLSPSHGIELSVGEKLVLNCTARTELNVGIDFNWEYPSSKHQHKKLVNRDLKTQSGSEMKKFLSTLTIDGVTRSDQGTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKTVAAPSVSDVPRDLEVVAATPTSLLISWDTHNAYNGYYRITYGETGGNSPVREFTVPHPEVTATISGLKPGVDDTITVYAVTNHHMPLRIFGPISINHRT

SEQ ID NO: 133 - protein, 368 aa, Artificial Sequence
  DOM15-26-593-ESBA105 dAb-scFv
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTKGPSDIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSS

SEQ ID NO: 134 - protein, 217 aa, Artificial Sequence
  DOM15-26-593-TNF adnectin
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTKGSPVSDVPRDLEVVAATPTSLLISWDTHNAYNGYYRITYGETGGNSPVREFTVPHPEVTATISGLKPGVDDTITVYAVTNHHMPLRIFGPISINHRT

SEQ ID NO: 135 - protein, 359 aa, Artificial Sequence
  DOM15-10-11-ESBA105 dAb-scFv
DIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKRTVAAPSDIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSS

SEQ ID NO: 136 - protein, 222 aa, Artificial Sequence
  DOM15-10-11-PEP1-5-19 dAb-dAb
DIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKRTVAAPSDIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 137 - protein, 208 aa, Artificial Sequence
  DOM15-10-11-TNF adnectin
DIQMTQSPSSLSASVGDRVTITCRASQWIGPELRWYQQKPGKAPKLLIYHTSILQSGVPSRFSGSGSGTDFTLTISSLQPEDFATYYCQQYMFQPMTFGQGTKVEIKRTVAAPSVSDVPRDLEVVAATPTSLLISWDTHNAYNGYYRITYGETGGNSPVREFTVPHPEVTATISGLKPGVDDTITVYAVTNHHMPLRIFGPISINHRT

SEQ ID NO: 138 - protein, 401 aa, Artificial Sequence
  VEGF anticalin-ESBA105
DGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPGTVAAPSDIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSS

SEQ ID NO: 139 - protein, 264 aa, Artificial Sequence
  VEGF anticalin-PEP1-5-19
DGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPGTVAAPSDIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 140 - protein, 250 aa, Artificial Sequence
  VEGF anticalin-TNF adnectin
DGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPGTVAAPSVSDVPRDLEVVAATPTSLLISWDTHNAYNGYYRITYGETGGNSPVREFTVPHPEVTATISGLKPGVDDTITVYAVTNHHMPLRIFGPISINHRT

SEQ ID NO: 141 - DNA, 1731 bp, Artificial Sequence
  Anti-TNF-alpha mAb (adalimumab) Fc disabled-DOM15-26-593 Heavy Chain with GSTVAAPSGS linker
gaggtgcagctggtggagtctggcggcggactggtgcagcccggcagaagcctgagactgagctgtgccgccagcggcttcaccttcgacgactacgccatgcactgggtgaggcaggcccctggcaagggcctggagtgggtgtccgccatcacctggaatagcggccacatcgactacgccgacagcgtggagggcagattcaccatcagccgggacaacgccaagaacagcctgtacctgcagatgaacagcctgagagccgaggacaccgccgtgtactactgtgccaaggtgtcctacctgagcaccgccagcagcctggactactggggccagggcaccctggtgacagtctcgagcgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggagcccaagagctgcgataagacccacacctgccccccctgccctgcccccgagctggccggcgcccctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagcctagagagccccaggtctacaccctgcctccctccagagatgagctgaccaagaaccaggtgtccctgacctgtctggtgaagggcttctaccccagcgacatcgccgtggagtgggagagcaacggccagcccgagaacaactacaagaccaccccccctgtgctggacagcgatggcagcttcttcctgtactccaagctgaccgtggacaagagcagatggcagcagggcaacgtgttcagctgcagcgtgatgcacgaggccctgcacaatcactacacccagaagagtctgagcctgtcccctggcaagggatccaccgtggccgctcccagcggatcagaggtgcagctgctggtgtctggcggcggactggtgcagcctggcggcagcctgagactgagctgcgccgccagcggcttcaccttcaaggcctaccccatgatgtgggtgcggcaggcccctggcaagggcctggaatgggtgtccgagatcagccccagcggcagctacacctactacgccgacagcgtgaagggccggttcaccatcagccgggacaacagcaagaacaccctgtacctgcagatgaacagcctgcgggccgaggacaccgccgtgtactactgcgccaaggacccccggaagctggactactggggccagggcaccctggtgaccgtgagcagc

SEQ ID NO: 142 - DNA, 1755 bp, Artificial Sequence
  Anti-TNF-alpha mAb (adalimumab) Fc disabled -DOM15-26-593 Heavy Chain with GS(TVAAPSGS) x2 linker
gaggtgcagctggtggagtctggcggcggactggtgcagcccggcagaagcctgagactgagctgtgccgccagcggcttcaccttcgacgactacgccatgcactgggtgaggcaggcccctggcaagggcctggagtgggtgtccgccatcacctggaatagcggccacatcgactacgccgacagcgtggagggcagattcaccatcagccgggacaacgccaagaacagcctgtacctgcagatgaacagcctgagagccgaggacaccgccgtgtactactgtgccaaggtgtcctacctgagcaccgccagcagcctggactactggggccagggcaccctggtgacagtctcgagcgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggagcccaagagctgcgataagacccacacctgccccccctgccctgcccccgagctggccggcgcccctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagcctagagagccccaggtctacaccctgcctccctccagagatgagctgaccaagaaccaggtgtccctgacctgtctggtgaagggcttctaccccagcgacatcgccgtggagtgggagagcaacggccagcccgagaacaactacaagaccaccccccctgtgctggacagcgatggcagcttcttcctgtactccaagctgaccgtggacaagagcagatggcagcagggcaacgtgttcagctgcagcgtgatgcacgaggccctgcacaatcactacacccagaagagtctgagcctgtcccctggcaagggatccacagtggctgcaccttccgggtcaaccgtcgccgcccccagcggaagcgaggtgcagctgctggtgtctggcggcggactggtgcagcctggcggcagcctgagactgagctgcgccgccagcggcttcaccttcaaggcctaccccatgatgtgggtgcggcaggcccctggcaagggcctggaatgggtgtccgagatcagccccagcggcagctacacctactacgccgacagcgtgaagggccggttcaccatcagccgggacaacagcaagaacaccctgtacctgcagatgaacagcctgcgggccgaggacaccgccgtgtactactgcgccaaggacccccggaagctggactactggggccagggcaccctggtgaccgtgagcagc

SEQ ID NO: 143 - DNA, 1779 bp, Artificial Sequence
  Anti-TNF-alpha mAb (adalimumab) Fc disabled -DOM15-26-593 Heavy Chain with GS(TVAAPSGS) x3 linker
gaggtgcagctggtggagtctggcggcggactggtgcagcccggcagaagcctgagactgagctgtgccgccagcggcttcaccttcgacgactacgccatgcactgggtgaggcaggcccctggcaagggcctggagtgggtgtccgccatcacctggaatagcggccacatcgactacgccgacagcgtggagggcagattcaccatcagccgggacaacgccaagaacagcctgtacctgcagatgaacagcctgagagccgaggacaccgccgtgtactactgtgccaaggtgtcctacctgagcaccgccagcagcctggactactggggccagggcaccctggtgacagtctcgagcgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggagcccaagagctgcgataagacccacacctgccccccctgccctgcccccgagctggccggcgcccctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagcctagagagccccaggtctacaccctgcctccctccagagatgagctgaccaagaaccaggtgtccctgacctgtctggtgaagggcttctaccccagcgacatcgccgtggagtgggagagcaacggccagcccgagaacaactacaagaccaccccccctgtgctggacagcgatggcagcttcttcctgtactccaagctgaccgtggacaagagcagatggcagcagggcaacgtgttcagctgcagcgtgatgcacgaggccctgcacaatcactacacccagaagagtctgagcctgtcccctggcaagggatccaccgtcgccgcaccaagcgggtcaacagtggccgctccctccggcagcactgtggctgcccccagcggaagcgaggtgcagctgctggtgtctggcggcggactggtgcagcctggcggcagcctgagactgagctgcgccgccagcggcttcaccttcaaggcctaccccatgatgtgggtgcggcaggcccctggcaagggcctggaatgggtgtccgagatcagccccagcggcagctacacctactacgccgacagcgtgaagggccggttcaccatcagccgggacaacagcaagaacaccctgtacctgcagatgaacagcctgcgggccgaggacaccgccgtgtactactgcgccaaggacccccggaagctggactactggggccagggcaccctggtgaccgtgagcagc

SEQ ID NO: 144 - DNA, 1803 bp, Artificial Sequence
  Anti-TNF-alpha mAb (adalimumab) Fc disabled -DOM15-26-593 Heavy Chain with GS(TVAAPSGS) x4 linker
gaggtgcagctggtggagtctggcggcggactggtgcagcccggcagaagcctgagactgagctgtgccgccagcggcttcaccttcgacgactacgccatgcactgggtgaggcaggcccctggcaagggcctggagtgggtgtccgccatcacctggaatagcggccacatcgactacgccgacagcgtggagggcagattcaccatcagccgggacaacgccaagaacagcctgtacctgcagatgaacagcctgagagccgaggacaccgccgtgtactactgtgccaaggtgtcctacctgagcaccgccagcagcctggactactggggccagggcaccctggtgacagtctcgagcgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggagcccaagagctgcgataagacccacacctgccccccctgccctgcccccgagctggccggcgcccctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagcctagagagccccaggtctacaccctgcctccctccagagatgagctgaccaagaaccaggtgtccctgacctgtctggtgaagggcttctaccccagcgacatcgccgtggagtgggagagcaacggccagcccgagaacaactacaagaccaccccccctgtgctggacagcgatggcagcttcttcctgtactccaagctgaccgtggacaagagcagatggcagcagggcaacgtgttcagctgcagcgtgatgcacgaggccctgcacaatcactacacccagaagagtctgagcctgtcccctggcaagggatccaccgtcgccgcaccaagcggatctaccgtcgcagccccttccgggtcaacagtggccgctccctccggcagcactgtggctgcccccagcggaagcgaggtgcagctgctggtgtctggcggcggactggtgcagcctggcggcagcctgagactgagctgcgccgccagcggcttcaccttcaaggcctaccccatgatgtgggtgcggcaggcccctggcaagggcctggaatgggtgtccgagatcagccccagcggcagctacacctactacgccgacagcgtgaagggccggttcaccatcagccgggacaacagcaagaacaccctgtacctgcagatgaacagcctgcgggccgaggacaccgccgtgtactactgcgccaaggacccccggaagctggactactggggccagggcaccctggtgaccgtgagcagc

SEQ ID NO: 145 - protein, 5 aa, Artificial Sequence
  Linker
PASGS

SEQ ID NO: 146 - protein, 6 aa, Artificial Sequence
  Linker
PDASGS

SEQ ID NO: 147 - protein, 7 aa, Artificial Sequence
  Linker
PDDASGS

SEQ ID NO: 148 - protein, 10 aa, Artificial Sequence
  Linker
GGGGSGGGGS

SEQ ID NO: 149 - protein, 15 aa, Artificial Sequence
  Linker
GGGGSGGGGSGGGGS

SEQ ID NO: 150 - protein, 14 aa, Artificial Sequence
  Linker
TVAAPSTVAAPSGS

SEQ ID NO: 151 - protein, 20 aa, Artificial Sequence
  Linker
TVAAPSTVAAPSTVAAPSGS

SEQ ID NO: 152 - protein, 42 aa, Artificial Sequence
  Linker
GSTVAAPSGSTVAAPSGSTVAAPSGSTVAAPSGSTVAAPSGS

SEQ ID NO: 153 - protein, 50 aa, Artificial Sequence
  Linker
GSTVAAPSGSTVAAPSGSTVAAPSGSTVAAPSGSTVAAPSGSTVAAPSGS

SEQ ID NO: 154 - protein, 8 aa, Artificial Sequence
  Linker
PAVPPPGS

SEQ ID NO: 155 - protein, 14 aa, Artificial Sequence
  Linker
PAVPPPPAVPPPGS

SEQ ID NO: 156 - protein, 20 aa, Artificial Sequence
  Linker
PAVPPPPAVPPPPAVPPPGS

SEQ ID NO: 157 - protein, 8 aa, Artificial Sequence
  Linker
TVSDVPGS

SEQ ID NO: 158 - protein, 14 aa, Artificial Sequence
  Linker
TVSDVPTVSDVPGS

SEQ ID NO: 159 - protein, 20 aa, Artificial Sequence
  Linker
TVSDVPTVSDVPTVSDVPGS

SEQ ID NO: 160 - protein, 8 aa, Artificial Sequence
  Linker
TGLDSPGS

SEQ ID NO: 161 - protein, 14 aa, Artificial Sequence
  Linker
TGLDSPTGLDSPGS

SEQ ID NO: 162 - protein, 20 aa, Artificial Sequence
  Linker
TGLDSPTGLDSPTGLDSPGS

SEQ ID NO: 163 - protein, 541 aa, Artificial Sequence
  BPC1801 (bispecific IGF1R-VEGFR2) heavy chain
QVQLVQSGAEVKKPGASVKVSCKASGYTFTDYYMNWVRQAPGQGLEWMGNINPNNGGTNYNQKFKDRVTMTTDTSTSTAYMELRSLRSDDTAVYYCARWILYYGRSKWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSEVVAATPTSLLISWRHPHFPTRYYRITYGETGGNSPVQEFTVPLQPPTATISGLKPGVDYTITVYAVTDGRNGRLLSIPISINYRT

SEQ ID NO: 164 - protein, 219 aa, Artificial Sequence
  BPC1801 (bispecific IGF1R-VEGFR2) light chain
DIVMTQSPLSLPVTPGEPASISCRSSQSIVQSNGDTYLEWYLQKPGQSPQLLIYRVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 477 aa, Artificial Sequence
  BPC1824 (CTLA4-Ig-anti-IL-13 dAb fusion)
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGVQLLESGGGLVQPGGSLRLSCAASGFVFPWYDMGWVRQAPGKGLEWVSSIDWHGKITYYADSVKGRFTISRDNSKNTLYLQMNSLRAEDTAVYYCATAEDEPGYDYWGQGTLVTVSS

(19) United States
(12) Patent Application Publication
(10) Pub. No.: US 2012/0076787 A1
(43) Pub. Date: Mar. 29, 2012

(54) COMBINATION OF A TNF-ALPHA ANTAGONIST AND A VEGF ANTAGONIST FOR USE IN THE TREATMENT OR PREVENTION OF DISEASES OF THE EYE

(76) Inventors: Peter Adamson, Hertfordshire (GB); Peter Franz Ertl, Hertfordshire (GB); Volker Germaschewski, Hertfordshire (GB); Gerald Wayne Gough, Hertfordshire (GB); Michael Steward, Hertfordshire (GB)

(21) Appl. No.: 13/322,213

(22) PCT Filed: May 26, 2010
(86) PCT No.: PCT/EP2010/057246

§ 371 Date: Nov. 23, 2011

Related U.S. Application Data
(60) Provisional application No. 61/181,887, filed on May 28, 2009.

(51) Int. Cl. A61K 39/395; A61P 27/02; C07K 16/46

(52) U.S. Cl. 424/136.1; 530/387.3

(57) ABSTRACT

The invention relates to combinations of TNFα antagonists with VEGF antagonists for use in treating diseases of the eye, and provides antigen-binding proteins which bind to TNFα or a TNFα receptor and/or VEGF or a VEGF receptor.

### BACKGROUND

Vision loss has become a major health problem for developed economies. Blindness or poor vision affects over 3 million US citizens over the age of 40 years and this increases significantly with age. For example, those aged 80 years old or greater comprise about 8% of the US population but nonetheless account for almost 70% of blindness. Eye diseases that are typically associated with age include age related macular degeneration (AMD), cataracts, diabetic macular edema, retinal vein occlusion (RVO) and glaucoma.

Age-related macular degeneration (AMD) is the leading cause of blindness in the developed world. There are two major clinical presentations of AMD. Atrophic (dry) AMD is characterised by the degeneration of retinal pigment epithelial (RPE) and neuroretina. The early stages of atrophic AMD are associated with the formation of drusen, under the RPE cell layer. Early atrophic AMD can progress to an end stage disease where the RPE degenerates completely and forms sharply demarcated areas of RPE atrophy in the region of the macula: “geographic atrophy”. In this form of the disease, the degeneration of RPE results in the secondary death of macular rods and cones and in these cases this leads to the severe age-related vision loss. A proportion of AMD patients develop what can either be regarded as a different form or a further complication of the disease. Approximately 10-20% of AMD patients develop choroidal neovascularisation (CNV). When this occurs the form of the disease is known as “wet AMD” and this can be associated with some of the most severe vision loss. In wet AMD, new choroidal vessels grow through breaks in Bruch's membrane and proliferate into and under the RPE and neuroretina. There are currently no definitive means of treatment for the very prevalent atrophic form of AMD nor to prevent the progression of early dry AMD either to geographic atrophy or to wet AMD, (Petrukhin K, Expert Opin Ther Targets (2007) 11: 625-639).

Diabetic macular edema (DME) is the most frequent cause of loss of reading vision in diabetic patients. The prevalence of DME in individuals who have had diabetes for 29 years or more is approximately 30% (Klein R et al Ophthalmology 1984: 91; 1464-1474). DME is associated with increased levels of IL-6, VEGF and other cytokines, with a generalised breakdown of the blood retinal barrier with leakage from abnormal retinal capillaries and microaneurysms developing in the sub retinal space. The goal of current DME treatment is to reduce the edema and leakage leading to improved visual acquity. Good glycemic control and laser photocoagulation or antiangiogenic treatment aim to prevent or delay further deterioration of the central macular region of the diabetic eye. Intravitreal injection of corticosteroids have also been used.

Retinal vein occlusion occurs subsequent to obstruction of the blood flow through a retinal vein. This might be due to clot formation or pressure increases in closely associated retinal arteries due to diabetes, glaucoma or high blood pressure. The reduced blood flow out of the retina leads to a generalised increase in blood pressure in ocular blood vessels and reduced oxygen levels in the eye. This in turn leads to abnormal blood vessel growth, hemorraging and edema, tissue damage and vision loss. There are two main forms of RVO, branch retinal vein occlusion (BRVO) and central retinal vein occlusion (CRVO). Sudden blurring or loss of vision is the common feature of RVO. Intraocular corticosteroids have been used to treat RVO, albeit with the associated risk of cataract development and raised intraocular pressure (Kiernan D F et al Exp Opinion in Pharmacotherapy 2009 10(15) 2511-2525). The prevalence of RVO ranges from ˜0.2% (CRVO) to ˜0.7% (BRVO).

Uveitis predominantly affects people of working age and comprises an inflammation of the uveal tract (iris, ciliary body and choroid). Anterior uveitis is the most common form of uveitis making up about 75% of uveitis cases and it and mainly affects the iris and ciliary body. Uveitis is regarded as an autoimmune disease and whilst the etiology remains unknown an association with HLA-B27 is present in about 50% of cases. Inflammation involving the posterior uveal tract (i.e. the choroid) is known as posterior uveitis and secondary involvement of the retina is common. Uveitis is predominantly an inflammatory disease with infiltration of CD4 T-cells into the ocular compartment (Paroli M P et al 2007 17(6) 938-942 Eur J Ophthalmology). Corticosteriods are again the mainstay for treatment either given topically, periocularly or systemically.

TNF-α (Tumour Necrosis Factor-α) is a pro-inflammatory cytokine which has been associated with a number of ophthalmic inflammatory conditions (Theodossiadis et al., Am. J. Ophthalmol. (2009) 147: 825-830).

VEGF (Vascular Endothelial Growth Factor) and VEGF-receptors are known to stimulate both choroidal and retinal vessel angiogenesis and regulate the vascular permeability of such vessels. (Gragoudas et al., N. Engl. J. Med (2004) 351: 2805) Neovascularisation and leakage are prominent features of the wet form of age-related macular degeneration. An aptamer, pegaptanib (Macugen™), which neutralises the VEGF-A isoform 165, and ranibizumab (Lucentis™) which blocks all isoforms of VEGF-A, have now been approved for use.

The inflammatory response also plays a significant pathophysiological role in neovascularisation (Sakuri et al., Invest Ophthalmol V is Sci (2003) 44: 5349-5354; Oh et al. Invest Ophthalmol V is Sci (1999) 40: 1891-1898; Shi et al., Exp Eye Res (2006) 83: 1325-1334.

Literature references relating to TNFα antagonists include Olson et al., Arch Opthalmol (2007) 125: 1221-1224; Shi et al., Exp Eye Res (2006) 83: 1325-1334 Kociok et al., Invest Ophthalmol V is Sci (2006) 11: 5057-5065Markomichelakis et al. Am J Ophthalmol (2005) 139: 537-540.

Studies indicate that intravitreal injections of infliximab may elicit a severe intracocular inflammatory reaction that appears to be dose related. Such adverse events were not seen with adalimumab (Program 4247, Poster D913, Intravitreal TNF inhibitors in the Treatment of Refractory Diabetic Macular Edema: A Pilot Study from the Pan American Collaborative Retina Study Group and Program 4749, Poster D1087, Ocular and Systemic Safety of Intravitreal TNF Inhibitors: A Pilot Study From the Pan American Collaborative Retina Study Group, The Association for Research in Vision and Ophthalmology (ARVO) May 2-6 2010. Ft. Lauderdale USA).

There is a need for treatment regimes which are effective at preventing ophthalmic disease progression and provide improved vision for a wider group of patients.

### SUMMARY OF INVENTION

The present invention relates to the combination of a TNFα antagonist and a VEGF antagonist, specifically for use in treating diseases of the eye.

Both anti-VEGF and anti-TNF approaches have a basis in treating AMD, and mechanistically these modalities may not overlap, such that a patient who does not respond successfully to an anti-VEGF approach therapy may respond to an anti-TNF treatment and vice versa.

The anti-inflammatory benefit of an anti-TNF combined with the anti-angiogenic activity of an anti-VEGF molecule will provide improved efficacy in treating such eye diseases.

The administration of a combination of an individual TNFα antagonist and an individual VEGF antagonist (i.e. separate TNFα and VEGF antagonist molecules) is covered by the present invention. In addition, the administration of a single construct with dual targeting functionality that acts as both a TNFα antagonist and a VEGF antagonist (i.e. able to bind to and inhibit, preferably block, the function of TNFα or a TNFα receptor, and bind to and inhibit, preferably block, the function of VEGF or a VEGF receptor) is covered by the present invention. The single construct may be based on an antibody scaffold or other such suitable scaffold. Receptor-Fc fusions are also considered part of the invention.

The present invention relates in particular to antigen binding proteins.

In particular, the present invention relates to a TNFα/VEGF dual targeting single construct wherein the TNFα antagonist portion is or is derived from a human anti-TNFα antibody. The TNFα antibody may be adalimumab or golimumab.

The present invention in particular relates to an antigen-binding protein comprising a protein scaffold which is linked to one or more epitope-binding domains wherein the antigen-binding protein has at least two antigen-binding sites at least one of which is from an epitope binding domain and at least one of which is from a paired V_{H}/V_{L} domain, and wherein at least one of the antigen-binding sites is capable of binding to TNFα or a TNFα Receptor e.g. TNFR1, and at least one of the antigen binding sites is capable of binding to VEGF or a VEGF Receptor, e.g. VEGFR2, for use in treating diseases of the eye.

A receptor-Fc fusion which is linked to one or more epitope-binding domains is also part of the invention e.g. a TNFα receptor-Fc fusion linked to a VEGF or VEGF receptor-binding domain, or a VEGF receptor-Fc fusion linked to a TNFα or a TNFα receptor-binding domain.

The present invention provides a dual targeting antigen binding molecule comprising a TNFα antagonist portion, a VEGF antagonist portion and a linker connecting said TNFα antagonist portion to said VEGF antagonist portion, wherein the TNFα antagonist portion comprises an amino acid sequence of any one of the TNFα antagonists listed in table 1; the VEGF antagonist portion comprises an amino acid sequence of any one of the VEGF antagonists listed in table 2; the linker is an amino acid sequence from 1-150 amino acids in length; and the dual targeting molecule is not DMS4000 or DMS4031. The linker may also be a non-peptide based linker, including, for example, polyethylene glycol (PEG) and PEG based linkers.

The invention also provides a polynucleotide sequence encoding an antigen binding protein of the invention e.g. a polynucleotide sequence encoding a heavy chain of any of the antigen-binding proteins described herein, and a polynucleotide encoding a light chain of any of the antigen-binding proteins described herein. Such polynucleotides represent the coding sequence which corresponds to the equivalent polypeptide sequences. However it will be understood that such polynucleotide sequences could be cloned into an expression vector along with a start codon, an appropriate signal sequence and a stop codon.

The invention also provides a recombinant transformed or transfected host cell comprising one or more polynucleotides encoding an antigen binding protein of the invention e.g. a heavy chain and a light chain of an antigen-binding protein described herein.

The invention further provides a method for the production of any of the antigen-binding proteins described herein which method comprises the step of culturing a host cell comprising at least one vector comprising a polynucleotide encoding an antigen binding protein of the invention, e.g. a first and second vector, said first vector comprising a polynucleotide encoding a heavy chain of an antigen-binding protein described herein and said second vector comprising a polynucleotide encoding a light chain of an antigen-binding protein described herein, in a suitable culture media, for example serum-free culture media.

The invention provides a pharmaceutical composition suitable for systemic delivery or topical delivery to the eye comprising an antigen-binding protein as described herein and a pharmaceutically acceptable carrier. The pharmaceutical composition of the invention may additionally comprise a further active agent.

The invention provides a TNFα antagonist selected from the group consisting of adalimumab, infliximab, etanercept, ESBA105, PEP1-5-19, PEP1-5-490, PEP1-5-493, an adnectin of SEQ ID NO:2, golimumab, certolizumab, ALK-6931, and an antibody comprising a heavy chain of SEQ ID NO:30 and a light chain of SEQ ID NO:31, for use in preventing or treating an eye disease, wherein the TNFα antagonist is to be administered in combination with a VEGF antagonist selected from the group consisting of bevacizumab, ranibizumab, r84, aflibercept, CT01, DOM15-10-11, DOM15-26-593, PRS-050, PRS-051, MP0012, CT-322, ESBA903, EPI-0030, EPI-0010 and DMS1571.

The invention also provides a VEGF antagonist selected from the group consisting of bevacizumab, ranibizumab, r84, aflibercept, CT01, DOM15-10-11, DOM15-26-593, PRS-050, PRS-051, MP0012, CT-322, ESBA903, EPI-0030, EPI-0010 and DMS1571, for use in preventing or treating an eye disease, wherein the VEGF antagonist is to be administered in combination with a TNFα antagonist selected from the group consisting of adalimumab, infliximab, etanercept, ESBA105, PEP1-5-19, PEP1-5-490, PEP1-5-493, an adnectin of SEQ ID NO:2, golimumab, certolizumab, ALK-6931, and an antibody comprising a heavy chain of SEQ ID NO:30 and a light chain of SEQ ID NO:31.

The invention also provides a dual targeting antigen binding molecule comprising a TNFα antagonist portion, a VEGF antagonist portion and a linker connecting said TNFα antagonist portion to said VEGF antagonist portion, wherein:
the TNFα antagonist portion comprises an amino acid sequence of any one of the TNFα antagonists listed in table 1;
the VEGF antagonist portion comprises an amino acid sequence of any one of the VEGF antagonists listed in table 2;
the linker is an amino acid sequence from 1-150 amino acids in length; and
the dual targeting molecule is not DMS4000 or DMS4031.

The invention also provides a dual targeting antigen binding molecule comprising a TNFα antagonist portion, a VEGF antagonist portion and a linker connecting said TNFα antagonist portion to said VEGF antagonist portion, wherein:
the TNFα antagonist portion comprises an amino acid sequence of any one of the TNFα antagonists listed in table 1;
the VEGF antagonist portion comprises an amino acid sequence of any one of the VEGF antagonists listed in table 2;
the linker is an amino acid sequence from 1-150 amino acids in length; and wherein the dual targeting antigen binding molecule is for use in preventing or treating a disease of the eye and is to be administered intravitreally every 4-6 weeks.

The invention also provides an antigen binding protein comprising the heavy chain sequence of SEQ ID NO:69, 70, 71 or 72 and the light chain sequence of SEQ ID NO:12.

A method of preventing or treating a patient afflicted with an eye disease comprising administering a prophylactically or therapeutically effective amount of a composition or dual targeting protein as disclosed herein systemically or topically to the eye of the patient is also provided.

### BRIEF DESCRIPTION OF THE FIGURES

FIG. 1 shows SDS-PAGE analysis of the anti-TNFα/anti-VEGF mAb-dAb, DMS4000.

FIG. 2 shows SEC profile of the anti-TNFα/anti-VEGF mAb-dAb, DMS4000.

FIG. 3 shows Anti-VEGF activity of DMS4000.

FIG. 4 shows Anti-TNFα activity of DMS4000.

FIG. 5 shows (PK) properties of DMS4000.

FIG. 6 shows the results of an ELISA and confirms that bispecific BPC1821 binds to both VEGFR2 and B7-1.

FIG. 7 shows the results of an ELISA and confirms that bispecific BPC1825 shows binding to both VEGF and B7-1.

FIG. 8 depicts a matrix for constructing dual-targeting antigen binding molecules of the invention.

FIG. 9 shows BIAcore analysis for the PEP-DOM construct

FIG. 10 shows BIAcore analysis for the PEP-DOM construct (close up of TNF/VEGF binding region of FIG. 9 binding curve)

FIG. 11 is a graphical representation of data presented in Table 10.

All compounds were administered by intravitreal injection in a volume of 2 μl. Black bars represent day 7 results. White bars represent day 14 results.

FIG. 12 is a graphical representation of data presented in Table 11.

All compounds were administered by intravitreal injection in a volume of 2 μl. Black bars represent day 7 results. White bars represent day 14 results.

FIG. 13 shows infrared (IR, upper left panel), autofluorescence (AF, lower left panel) and fluorescien angiography (FS, large panel) at 7 (FS 1st) and 14 days (FS 2nd) after laser PC—showing example images. 1. Vehicle treated eyes, 2. eyes treated with 2 μg DMS1571 and 8. eyes treated with 30 μg Enbrel™. It is notable that the CNV lesions appear more punctuate and less diffuse than lesions responding to treatment with DMS1571.

FIG. 14 is a graphical representation of data presented in Table 12.

All compounds were administered by intravitreal injection in a volume of 2 μl

FIG. 15 shows example photomicrographs of flat-mounted retinae stained with ED1 mab. Panels 1A-1B and panel Enbrel 8.4 show flat-mounts of retinas from eyes treated with anti-VEGF (DMS1571) (1A), Vehicle only (1B) or Enbrel (Enbrel 8.4). Macrophages, associated with laser burn site, visualised with ED1 (CD 68, black) X20. Panel 1D shows a Cryostat section (20 μm) of retina showing macrophages (ED1+, black) associated with laser burn site which has penetrated to the inner nuclear layer (INL) of the retina. RGC, retinal ganglion cell layer; BV, blood vessel. x20.

### DEFINITIONS

The term ‘Protein Scaffold’ as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. Such protein scaffolds may comprise antigen-binding sites in addition to the one or more constant regions, for example where the protein scaffold comprises a full IgG. Such protein scaffolds will be capable of being linked to other protein domains, for example protein domains which have antigen-binding sites, for example epitope-binding domains or ScFv domains.

The term ‘receptor-Fc fusion’ as used herein refers to a soluble ligand or extracellular domain of a receptor or cell surface protein linked to the Fc region of an antibody. Fragments of such soluble ligands or extracellular domains of a receptor or cell surface protein are included within this definition providing they retain the biological function of the full length protein, i.e. providing they retain antigen-binding ability. A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. An “antibody single variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

A “humanised antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl. Acad Sci USA, 86:10029-10032 (1989), Hodgson et al. Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951. In an embodiment, an antibody of the invention is a humanised antibody.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

A “CDR variant” includes an amino acid sequence modified by at least one amino acid, wherein said modification can be chemical or a partial alteration of the amino acid sequence (for example by no more than 10 amino acids), which modification permits the variant to retain the biological characteristics of the unmodified sequence. For example, the variant is a functional variant which binds to and neutralises IL-18. A partial alteration of the CDR amino acid sequence may be by deletion or substitution of one to several amino acids, or by addition or insertion of one to several amino acids, or by a combination thereof (for example by no more than 10 amino acids). The CDR variant may contain 1, 2, 3, 4, 5 or 6 amino acid substitutions, additions or deletions, in any combination, in the amino acid sequence. The CDR variant or binding unit variant may contain 1, 2 or 3 amino acid substitutions, insertions or deletions, in any combination, in the amino acid sequence. The substitutions in amino acid residues may be conservative substitutions, for example, substituting one hydrophobic amino acid for an alternative hydrophobic amino acid. For example leucine may be substituted with valine, or isoleucine.

The term “human antibody” refers to an antibody derived from human immunoglobulin gene sequences. These fully human antibodies provide an alternative to re-engineered, or de-immunized, rodent monoclonal antibodies (e.g. humanised antibodies) as a source of low immunogenicity therapeutic antibodies and they are normally generated using either phage display or transgenic mouse platforms In an embodiment, an antibody of the invention is a human antibody.

The phrase “immunoglobulin single variable domain” refers to an antibody variable domain (V_{H}, V_{HH}, V_{L}) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are still considered to be “domain antibodies” according to the invention. As used herein “V_{H} includes camelid V_{HH} domains. NARV are another type of immunoglobulin single variable domain which were identified in cartilaginous fish including the nurse shark. These domains are also known as Novel Antigen Receptor variable region (commonly abbreviated to V(NAR) or NARV). For further details see Mol. Immunol. (2006) 44: 656-665 and US20050043519A.

The term “Epitope-binding domain” refers to a domain that specifically binds an antigen or epitope independently of a different V region or domain, this may be a domain antibody (dAb), for example a human, camelid or shark immunoglobulin single variable domain or it may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which have been subjected to protein engineering in order to obtain binding to a ligand other than the natural ligand.

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods (2001) 248 (1-2): 31-45.

Lipocalins are a family of extracellular proteins which transport small hydrophobic molecules such as steroids, bilins, retinoids and lipids. They have a rigid (3-sheet secondary structure with a number of loops at the open end of the conical structure which can be engineered to bind to different target antigens. Anticalins are between 160-180 amino acids in size, and are derived from lipocalins. For further details see Biochim Biophys Acta (2000) 1482: 337-350, U.S. Pat. No. 7,250,297B1 and US20070224633.

An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. (2004) 17: 455-462 and EP1641818A1.

Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology (2205) 23(12): 1556-1561 and Expert Opinion on Investigational Drugs (June 2007) 16(6): 909-917.

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem. (1999) 274: 24066-24073.

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomising residues in the first α-helix and a β-turn of each repeat. Their binding interface can be increased by increasing the number of modules (a method of affinity maturation). For further details see J. Mol. Biol. (2003) 332: 489-503; PNAS (2003) 100(4): 1700-1705; and J. Mol. Biol. (2007) 369: 1015-1028 and US20040132028A1.

Fibronectin is a scaffold which can be engineered to bind to antigen. Adnectins consists of a backbone of the natural amino acid sequence of the 10th domain of the repeating units of human fibronectin type III (FN3). Three loops at one end of the β-sandwich can be engineered to enable an Adnectin to specifically recognize a therapeutic target of interest. For further details see Protein Eng. Des. Sel. (2005) 18: 435-444, US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. (2005) 5: 783-797.

Microbodies are derived from naturally occurring microproteins of 25-50 amino acids in length which contain 3-4 cysteine bridges—examples of microproteins include KalataB1 and conotoxin and knottins. The microproteins have a loop which can be engineered to include up to 25 amino acids without affecting the overall fold of the microprotein. For further details of engineered knottin domains, see WO2008098796.

Other epitope binding domains include proteins which have been used as a scaffold to engineer different target antigen binding properties including human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins), as reviewed in Chapter 7—Non-Antibody Scaffolds from Handbook of Therapeutic Antibodies (2007, edited by Stefan Dubel) and Protein Science (2006) 15:14-27. Epitope binding domains of the present invention could be derived from any of these alternative protein domains.

A “dual variable domain immunoglobulin (DVD-Ig)” is a dual-specific, tetravalent immunoglobulin G (IgG)-like molecule (Wu et al. Nature Biotechnology (2007) 25: 1290-1297). A DVD-Ig can be defined as a binding protein comprising a polypeptide chain, wherein said polypeptide chain comprises VDI-(XI)n-VD2-C—(X2)n, wherein VDI is a first variable domain, VD2 is a second variable domain, C is a constant domain, X1 represents an amino acid or polypeptide (linker), X2 represents an Fc region and n is 0 or 1 (WO 2007024715). In the context of the present invention VDI binds to TNFα or a TNFα receptor, and VD2 binds to VEGF or a VEGF receptor, or vice versa.

As used herein, the terms “paired V_{H} domain”, “paired V_{L} domain”, and “paired V_{H}/V_{L} domain(s)” refer to antibody variable domains which specifically bind antigen only when paired with their partner variable domain. There is always one V_{H} and one V_{L} in any pairing, and the term “paired V_{H} domain” refers to the V_{H} partner, the term “paired V_{L} domain” refers to the V_{L} partner, and the term “paired V_{H}/V_{L} domain(s)” refers to the two domains together.

The term “antigen binding protein” as used herein refers to antibodies, antibody fragments, for example a domain antibody (dAb), ScFv, FAb, FAb₂, and other protein constructs, such as receptor-Fc fusions, which are capable of binding to TNFα and/or VEGF. Antigen binding molecules may comprise at least one Ig variable domain, for example antibodies, domain antibodies, multiples of domain antibodies e.g. dumbbells, dAb-dAb in-line fusions, Fab, Fab′, F(ab′)2, Fv, ScFv, diabodies, mAbdAbs, DVD-Igs, affibodies, heteroconjugate antibodies or bispecifics, including a bispecific antibody with a first specificity for TNFα or a TNFα receptor and a second specificity for VEGF or a VEGF receptor. In one embodiment the antigen binding molecule is an antibody. In another embodiment the antigen binding molecule is a dAb, i.e. an immunoglobulin single variable domain such as a V_{H}, V_{HH} or V_{L} that specifically binds an antigen or epitope independently of a different V region or domain. Antigen binding molecules may be capable of binding to two targets, i.e. they may be dual targeting proteins. Antigen binding molecules may be a combination of antibodies and antigen binding fragments such as for example, one or more domain antibodies and/or one or more ScFvs linked to a monoclonal antibody. Antigen binding molecules may also comprise a non-Ig domain for example a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which have been subjected to protein engineering in order to obtain binding to TNFα and/or VEGF. As used herein “antigen binding protein” will be capable of antagonising and/or neutralising human TNFα and/or VEGF. In addition, an antigen binding protein may block TNFα and/or VEGF activity by binding to TNFα and/or VEGF and preventing a natural ligand from binding and/or activating the receptor.

As used herein “VEGF antagonist” includes any compound capable of reducing and/or eliminating at least one activity of VEGF. By way of example, a VEGF antagonist may bind to VEGF and that binding may directly reduce or eliminate VEGF activity or it may work indirectly by blocking at least one ligand from binding the receptor.

As used herein “TNFα antagonist” includes any compound capable of reducing and/or eliminating at least one activity of TNFα. By way of example, a TNFα antagonist may bind to TNFα and that binding may directly reduce or eliminate TNFα activity or it may work indirectly by blocking at least one ligand from binding the receptor.

The term “specifically binds” as used in relation to antigen binding proteins means that the antigen binding protein binds to it's target protein(s) (e.g. TNFα, TNFR, BEGF, VEGFR) with no or insignificant binding to other (for example, unrelated) proteins. The term, however, does not exclude the fact that an antibody to a target protein in a given species (e.g. human) may also be cross-reactive with other forms of the target protein in other species (e.g. a non-human primate).

The term “KD” refers to the equilibrium dissociation constant. In one embodiment of the invention the antigen-binding site binds to antigen with a KD of at most 1 mM, for example a KD of 10 nM, 1 nM, 500 pM, 200 pM, 100 pM, to each antigen as measured by Biacore™. In one embodiment of the invention the antigen-binding site binds to antigen with a KD 10 nM or less, 1 nM or less, 500 pM or less, 200 pM or less, 100 pM or less, to each antigen as measured by Biacore™.

As used herein, the term “antigen-binding site” refers to a site on a construct which is capable of specifically binding to antigen, this may be a single domain, for example an epitope-binding domain, or it may be paired V_{H}/V_{L} domains as can be found on a standard antibody. In some aspects of the invention single-chain Fv (ScFv) domains can provide antigen-binding sites.

The terms “mAb/dAb” and dAb/mAb” are used herein to refer to antigen-binding proteins of the present invention. The two terms can be used interchangeably, and are intended to have the same meaning as used herein.

The term “constant heavy chain 1” is used herein to refer to the constant domain of an immunoglobulin heavy chain, C_{H}1.

The term “constant light chain” is used herein to refer to the constant domain of an immunoglobulin light chain, C_{L}.

The term “library” refers to a mixture of heterogeneous polypeptides or nucleic acids. The library is composed of members, each of which has a single polypeptide or nucleic acid sequence. To this extent, “library” is synonymous with “repertoire.”

A “universal framework” is a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, J. Mol. Biol. (1987) 196: 910-917.

### DETAILED DESCRIPTION OF INVENTION

The present invention provides compositions comprising a TNFα antagonist and/or a VEGF antagonist suitable for use in the eye. The present invention also provides the combination of a TNFα antagonist and a VEGF antagonist, for use in preventing or treating diseases of the eye. The present invention also provides a method of preventing or treating diseases of the eye by administering a TNFα antagonist in combination with a VEGF antagonist. The TNFα antagonist and the VEGF antagonist may be administered separately, sequentially or simultaneously.

The administration of a combination of an individual TNFα antagonist and an individual VEGF antagonist (i.e. separate TNFα and VEGF antagonist molecules) is covered by the present invention. In addition, the administration of a single molecule or construct capable of binding to two or more antigens is covered by the present invention e.g. a molecule with dual targeting functionality (i.e. able to bind to and inhibit, preferably block, the function of TNFα or a receptor for TNFα, and bind to and inhibit, preferably block, the function of VEGF or a receptor for VEGF) that acts as both a TNFα antagonist and a VEGF antagonist, is covered by the present invention. For example, the invention provides a dual targeting molecule which is capable of binding to TNFα and VEGFR2, and so forth. In an embodiment the dual targeting molecule is capable of binding to a TNF receptor and a VEGF receptor.

The TNFα antagonist of the invention may inhibit signalling through a TNF receptor by 30%, 40%, 50%, 60%, 70%, 80%, 85%, 90%, 95%, 98% or 100%. The VEGF antagonist of the invention may inhibit signalling through a VEGF receptor by 30%, 40%, 50%, 60%, 70%, 80%, 85%, 90%, 95%, 98% or 100%.

In an embodiment the TNFα antagonist is a human antigen binding protein, in particular a human anti-TNFα antibody or fragment thereof, or a human anti-TNFR antibody or fragment thereof. In an embodiment the VEGF antagonist is a human antigen binding protein, in particular a human anti-VEGF antibody or fragment thereof, or a human anti-VEGFR antibody or fragment thereof. In an embodiment the antigen binding protein is a TNFα/VEGF dual targeting single construct wherein the TNFα antagonist portion is human. In a particular embodiment, the TNF antagonist is or is derived from adalimumab or golimumab.

The antagonists may be based on an antibody scaffold or other such suitable scaffold as described herein. Such antagonists may be antibodies or epitope binding domains for example dAbs. Receptor-Fc fusions are considered part of the invention.

The antagonists of the invention may be co-administered as a mixture of separate molecules which are administered at the same time (simultaneously), or are administered within a specified period of each other (sequentially), for example within a month, a week or within 24 hours of each other, for example within 20 hours, or within 15 hours or within 12 hours, or within 10 hours, or within 8 hours, or within 6 hours, or within 4 hours, or within 2 hours, or within 1 hour, or within 30 minutes of each other. The antagonists of the invention may be co-administered as separate formulations or as a single formulation, e.g. liposomes containing both antagonists.

TNFα antagonists within the scope of the invention, which may be administered in combination with a VEGF antagonist of the invention, or which may be used in generating dual targeting molecules of the invention, include those listed below in table 1.

TABLE 1
TNFα antagonists

| Name | Format | SEQ ID NO |
|---|---|---|
| Adalimumab (Humira ™) | Human mAb | 10 (heavy chain) |
| | | 12 (light chain) |
| Infliximab (Remicade ™) | Chimaeric mAb | 32 (heavy chain) |
| | | 33 (light chain) |
| Etanercept (Enbrel ™) | TNF Receptor-Fc | 34 |
| | fusion | |
| ESBA105 | Humanised scFv | 38 |
| PEP1-5-19 | Human Vκ dAb | 35 |
| PEP1-5-490 | Human Vk dAb | 36 |
| PEP1-5-493 | Human Vk dAb | 37 |
| — | Adnectin | 2 |
| Golimumab (Simponi ™) | Human mAb | — |
| Certolizumab (Cimiza ™) | Humanised Fab | — |
| | (PEGylated) | |
| ALK-6931 | TNF Receptor- | — |
| | Fc(IgG1) fusion | |

In addition to the TNFα antagonists identified by name in Table 1, a TNFα antagonist according to the invention includes an antibody comprising a heavy chain of SEQ ID NO:30 and a light chain of SEQ ID NO: 31.

VEGF antagonists within the scope of the invention, which may be administered in combination with a TNFα antagonist of the invention, or which may be used in generating dual targeting molecules of the invention, include those listed below in table 2.

TABLE 2
VEGF antagonists

| Name | Format | SEQ ID NO |
|---|---|---|
| Bevacizumab | Humanised mAb | 22 (heavy chain) |
| (Avastin ™) | | 21 (light chain) |
| Ranibizumab | Humanised Fab | 39 (heavy chain) |
| (Lucentis ™) | | 40 (light chain) |
| r84 | Humanised mAb | 41 (VH) |
| | | 42 (VL) |
| Aflibercept | Receptor-Fc fusion | 43 |
| (VEGF-Trap) | | |
| CT01 | Adnectin | 45 |
| DOM15-10-11 | Human Vκ dAb | 44 |
| DOM15-26-593 | Human Vκ dAb | 1 |
| PRS-050 | Anticalin | — |
| PRS-051 | Anticalin | — |
| MP0112 | Darpin | — |
| CT-322 | Humanised scFv | — |
| ESBA903 | Humanised scFv | — |
| EPI-0030 | Humanised mAb | — |
| EPI-0010 | Humanised mAb | — |
| DMS1571 | Fc formatted version of | 65 |
| | DOM15-26-593 human Vκ | |
| | dAb (exists as a dimer | |
| | of this sequence) | |

The present invention provides an antigen-binding protein for use in treating diseases of the eye comprising a protein scaffold which is linked to one or more epitope-binding domains wherein the antigen-binding protein has at least two antigen-binding sites at least one of which is from an epitope binding domain and at least one of which is from a paired V_{H}/V_{L} domain and wherein at least one of the antigen-binding sites binds to TNFα, or a receptor for TNFα, and at least one of the antigen-binding sites binds to VEGF, or a receptor for VEGF.

Such antigen-binding proteins comprise a protein scaffold, for example an Ig scaffold such as IgG, for example a monoclonal antibody, which is linked to one or more epitope-binding domains, for example a domain antibody, wherein the binding protein has at least two antigen-binding sites, at least one of which is from an epitope binding domain, and wherein at least one of the antigen-binding sites binds to TNFα, or a receptor for TNFα, at least one of the antigen-binding sites binds to VEGF, or a receptor for VEGF, and to methods of producing and uses thereof, particularly uses in ocular therapy.

Such antigen-binding proteins of the present invention are also referred to as mAbdAbs.

In one embodiment the protein scaffold of the antigen-binding protein of the present invention is an Ig scaffold, for example an IgG scaffold or IgA scaffold. The IgG scaffold may comprise all the domains of an antibody (i.e. C_{H}1, C_{H}2, C_{H}3, V_{H}, V_{L}, C_{L}). The antigen-binding protein of the present invention may comprise an IgG scaffold selected from IgG1, IgG2, IgG3, IgG4 or IgG4PE.

The antigen-binding protein of the present invention has at least two antigen-binding sites, for example it has two binding sites, for example where the first binding site has specificity for a first epitope on an antigen and the second binding site has specificity for a second epitope on the same antigen. In a further embodiment there are 4 antigen-binding sites, or 6 antigen-binding sites, or 8 antigen-binding sites, or 10 or more antigen-binding sites. In one embodiment the antigen-binding protein has specificity for more than one antigen, for example two antigens, or for three antigens, or for four antigens.

In another aspect, the invention relates to an antigen-binding protein which is capable of binding to TNFα, or a TNFα receptor, and VEGF, or a VEGF receptor, comprising at least one homodimer comprising two or more structures of formula I:

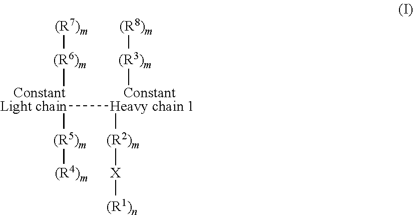

wherein
X represents a constant antibody region comprising constant heavy domain 2 (C_{H}2) and constant heavy domain 3 (C_{H}3);
R¹, R⁴, R⁷ and R⁸ each represent an epitope-binding domain;
R² represents a domain selected from the group consisting of constant heavy chain 1 (C_{H}1), and an epitope-binding domain;
R³ represents a domain selected from the group consisting of a paired V_{H} and an epitope-binding domain;
R⁵ represents a domain selected from the group consisting of constant light chain (CO, and an epitope-binding domain;
R⁶ represents a domain selected from the group consisting of a paired V_{L} and an epitope-binding domain;
n represents an integer independently selected from: 0, 1, 2, 3 and 4;
m represents an integer independently selected from: 0 and 1,
wherein the Constant Heavy chain 1 (C_{H}1) and the Constant Light chain (CO domains are associated;
wherein at least one epitope binding domain is present;
and when R³ represents a paired V_{H} domain, R⁶ represents a paired V_{L} domain, so that the two domains are together capable of binding antigen.
In one embodiment R⁶ represents a paired V_{L} and R³ represents a paired V_{H}.
In a further embodiment either one or both of R⁷ and R⁸ represent an epitope binding domain.
In yet a further embodiment either one or both of R¹ and R⁴ represent an epitope binding domain.
In one embodiment R⁴ is present.
In one embodiment R¹, R⁷ and R⁸ represent an epitope binding domain.
In one embodiment R¹, R⁷ and R⁸, and R⁴ represent an epitope binding domain.
In one embodiment (R¹)ₙ, (R²)ₘ, (R⁴)ₘ and (R⁵)ₘ=0, i.e. are not present, R³ is a paired V_{H} domain, R⁶ is a paired V_{L} domain, R⁸ is a V_{H} dAb, and R⁷ is a V_{L} dAb.
In another embodiment (R¹)ₙ, (R²)ₘ, (R⁴)ₘ and (R⁶)ₘ are 0, i.e. are not present, R³ is a paired V_{H} domain, R⁶ is a paired V_{L} domain, R⁸ is a V_{H} dAb, and (R⁷)ₘ=0 i.e. not present.
In another embodiment (R²)ₘ, and (R⁵)ₘ are 0, i.e. are not present, R¹ is a dAb, R⁴ is a dAb, R³ is a paired V_{H} domain, R⁶ is a paired V_{L} domain, (R⁸)ₘ and (R⁷)ₘ=0 i.e. not present.

In one embodiment of the present invention the epitope binding domain is a dAb.

In another aspect of the invention, the antigen binding protein is a bispecific antibody having a first specificity for TNFα or a TNFα receptor, and a second specificity for VEGF or a VEGF receptor.

In a further aspect of the invention, the antigen binding protein is a dual variable domain immunoglobulin (DVD-Ig).

In another aspect of the invention, the antigen binding protein is a dAb-dAb in-line fusion.

In another aspect of the invention, the antigen binding protein is a Receptor-Fc fusion, which may be linked to one or more epitope-binding domains. Receptor-Fc fusions comprise an immunoglobulin scaffold i.e. they comprise the Fc portion of an antibody, which is linked to a soluble ligand or extracellular domain of a receptor or cell surface protein and one or more epitope binding domains. Such receptor-Fc-epitope binding domain fusions may also be referred to as receptor-Ig-epitope binding domain fusions. The Fc portion may be selected from antibodies of any isotype, for example IgG1, IgG2, IgG3, IgG4 or IgG4PE.

In one embodiment the antigen-binding proteins of the invention have specificity for VEGF, for example they comprise a receptor-Fc fusion linked to an epitope binding domain which binds to VEGF, for example a dAb, an anticalin, or an adnectin which binds to VEGF.

In one embodiment the antigen-binding proteins of the invention have specificity for VEGFR2, for example they comprise a receptor-Fc fusion linked to an epitope binding domain which binds to VEGFR2, for example a dAb or an adnectin which binds to VEGFR2.

In one embodiment the antigen-binding proteins of the invention have specificity for TNFα, for example they comprise a receptor-Fc fusion linked to an epitope binding domain which binds to TNFα, for example a dAb or an adnectin which binds to TNFα.

In an embodiment the antigen binding proteins of the invention have specificity for both TNFα or a TNFα receptor, and VEGF or a VEGF receptor, for example they comprise a TNFα receptor-Fc fusion linked to an epitope binding domain which binds to VEGF or a VEGF receptor. Another example, is an antigen binding protein that comprises a VEGF receptor-Fc fusion linked to an epitope binding domain which binds to TNFα or a TNFα receptor.

It will be understood that any of the antigen-binding proteins described herein will be capable of neutralising one or more antigens, for example they will be capable of neutralising TNFα and/or they will also be capable of neutralising VEGF.

The term “neutralises” and grammatical variations thereof as used throughout the present specification in relation to antigen-binding proteins of the invention means that a biological activity of the target is reduced, either totally or partially, in the presence of the antigen-binding proteins of the present invention in comparison to the activity of the target in the absence of such antigen-binding proteins.

Neutralisation may be due to but not limited to one or more of blocking ligand binding, preventing the ligand activating the receptor, down regulating the receptor or affecting effector functionality.

Levels of neutralisation can be measured in several ways, for example in an IL-8 secretion assay in MRC-5 cells which may be carried out for example as described in Example 1.3. The neutralisation of TNFα in this assay is measured by assessing the inhibition of IL-8 secretion in the presence of neutralising antigen-binding protein. Levels of neutralisation could also be measured in an assay which measures inhibition of ligand binding to receptor which may be carried out for example as described in Example 1.3. The neutralisation of VEGF, in this assay is measured by assessing the decreased binding between the ligand and its receptor in the presence of neutralising antigen-binding protein.

Other methods of assessing neutralisation, for example, by assessing the decreased binding between the ligand and its receptor in the presence of neutralising antigen-binding protein are known in the art, and include, for example, Biacore™ assays.

In an alternative aspect of the present invention there is provided antigen-binding proteins which have at least substantially equivalent neutralising activity to the antigen binding proteins exemplified herein.

The antigen-binding proteins of the invention have specificity for TNFα or TNFα receptor, for example they comprise an epitope-binding domain which is capable of binding to TNFα, and/or they comprise a paired V_{H}/V_{L} which binds to TNFα. The antigen-binding protein may comprise an antibody which is capable of binding to TNFα. The antigen-binding protein may comprise a dAb which is capable of binding to TNFα.

The antigen-binding protein of the present invention also has specificity for VEGF or a receptor for VEGF. In one embodiment the antigen-binding protein of the present invention is capable of binding TNFα and VEGF simultaneously.

It will be understood that any of the antigen-binding proteins described herein may be capable of binding two or more antigens simultaneously, for example, as determined by stochiometry analysis by using a suitable assay such as that described in Example 3.

Examples of such antigen-binding proteins include VEGF antibodies which have an epitope binding domain which is a TNFα antagonist, for example an anti-TNFα adnectin, attached to the c-terminus or the n-terminus of the heavy chain or the c-terminus or n-terminus of the light chain. Examples include an antigen binding protein comprising the heavy chain sequence set out in SEQ ID NO: 20 or 22 and the light chain sequence set out in SEQ ID NO: 21, wherein one or both of the Heavy and Light chain further comprise one or more epitope-binding domains which bind to TNFα, for example an epitope binding domain selected from those set out in SEQ ID NO: 2 and SEQ ID NO: 17.

In one embodiment the antigen-binding protein will comprise an anti-VEGF antibody linked to an epitope binding domain which is a TNFα antagonist, wherein the anti-VEGF antibody has the same CDRs as the antibody which has the heavy chain sequence of SEQ ID NO:20 or 22, and the light chain sequence of SEQ ID NO: 21.

Examples of such antigen-binding proteins include TNFα antibodies which have an epitope binding domain which is a VEGF antagonist attached to the c-terminus or the n-terminus of the heavy chain or the c-terminus. Examples include an antigen binding protein comprising the heavy chain sequence set out in SEQ ID NO: 10 and the light chain sequence set out in SEQ ID NO: 12 wherein one or both of the Heavy and Light chain further comprise one or more epitope-binding domains which is capable of antagonising VEGF, for example by binding to VEGF or to a VEGF receptor for example VEGFR2. Such epitope-binding domains can be selected from those set out in SEQ ID NO: 1, 18, 19, 23 or 44.

In one embodiment the antigen binding constructs of the present invention comprise the heavy chain sequence of SEQ ID NO: 14 and the light chain sequence of SEQ ID NO: 12, or the heavy chain sequence of SEQ ID NO: 15 and the light chain sequence of SEQ ID NO: 12, or the heavy chain sequence of SEQ ID NO: 24 and the light chain sequence of SEQ ID NO: 12.

In an embodiment, the antigen binding constructs of the present invention comprise an anti-TNFα binding protein as disclosed in WO0212502, US2007/0003548, U.S. Pat. No. 7,250,165, EP01309691, or WO0212500, all of which are herein incorporated by reference in their entirety.

In one embodiment the antigen-binding protein will comprise an anti-TNFα antibody linked to an epitope binding domain which is a VEGF antagonist, wherein the anti-TNFα antibody has the same CDRs as the antibody which has the heavy chain sequence of SEQ ID NO:10, and the light chain sequence of SEQ ID NO: 12.

Other examples of such antigen-binding proteins include anti-TNFα antibodies which have an anti-VEGF epitope binding domain, attached to the c-terminus or the n-terminus of the heavy chain or the c-terminus or n-terminus of the light chain wherein the VEGF epitope binding domain is a VEGF dAb which is selected from any of the VEGF dAb sequences which are set out in WO2007080392 (which is incorporated herein by reference), in particular the dAbs which are set out in SEQ ID NO:117, 119, 123, 127-198, 539 and 540; or a VEGF dAb which is selected from any of the VEGF dAb sequences which are set out in WO2008149146 (which is incorporated herein by reference), in particular the dAbs which are described as DOM15-26-501, DOM15-26-555, DOM15-26-558, DOM15-26-589, DOM15-26-591, DOM15-26-594 and DOM15-26-595, or a VEGF dAb which is selected from any of the VEGF dAb sequences which are set out in WO2007066106 (which is incorporated herein by reference), or a VEGF dab which is selected from any of the VEGF dAb sequences which are set out in WO 2008149147 (which is incorporated herein by reference) or a VEGF dab which is selected from any of the VEGF dAb sequences which are set out in WO 2008149150 (which is incorporated herein by reference).

These specific sequences and related disclosures in WO2007080392, WO2008149146, WO2007066106, WO2008149147 and WO 2008149150 are incorporated herein by reference as though explicitly written herein with the express intention of providing disclosure for incorporation into claims herein and as examples of variable domains and antagonists for application in the context of the present invention.

Other examples of such antigen-binding constructs include anti-VEGF antibodies which have one or more anti-TNFalpha epitope binding domains, attached to the c-terminus or the n-terminus of the heavy chain or the c-terminus or n-terminus of the light chain wherein the TNFalpha epitope binding domain is a TNF-alpha dAb which is selected from any of the TNFalpha dAbs disclosed in WO04003019 (which is incorporated herein by reference), in particular the dAbs which are described as TAR1-5-19, TAR1-5, and TAR1-27. These specific sequences and related disclosures in WO04003019 are incorporated herein by reference as though explicitly written herein with the express intention of providing disclosure for incorporation into claims herein and as examples of variable domains and antagonists for application in the context of the present invention.

Other examples of such antigen-binding constructs include anti-VEGF antibodies which have one or more anti-TNFR1 epitope binding domains, attached to the c-terminus or the n-terminus of the heavy chain or the c-terminus or n-terminus of the light chain wherein the TNFR1 epitope binding domain is a TNFR1 dAb which is selected from any of the TNFR1 dAb sequences in WO04003019 (which is incorporated herein by reference), in particular the dAbs which are described as TAR2-10, and TAR2-5; or a TNFR1 dAb which is selected from any of the TNFR1 dAb sequences in WO2006038027 (which is incorporated herein by reference), in particular the dAbs which are set out in SEQ ID NO: 32-98, 167-179, 373-401, 431, 433-517 and 627; or a TNFR1 dAb which is selected from any of the TNFR1 dAb sequences in WO2008149144 (which is incorporated herein by reference), in particular the dAbs which are described as DOM1h-131-511, DOM1h-131-201, DOM1h-131-202, DOM1h-131-203, DOM1h-131-204, DOM1h-131-205; or a TNFR1 dAb which is selected from any of the TNFR1 dAb sequences in WO2008149148 (which is incorporated herein by reference), in particular the dAb which is described as DOM1h-131-206.

These specific sequences and related disclosures in WO2006038027 and WO2008149144 are incorporated herein by reference as though explicitly written herein with the express intention of providing disclosure for incorporation into claims herein and as examples of variable domains and antagonists for application in the context of the present invention.

Further examples of antigen-binding proteins include TNFR2-Ig fusions linked to an epitope binding domain with a specificity for VEGFR2, for example an anti-VEGFR2 adnectin, linked to the c-terminus or the n-terminus of the TNFR2-Ig fusion, for example an antigen-binding protein comprising the TNFR2-Ig sequence set out in SEQ ID NO:34 which further comprises one or more epitope-binding domains which bind to VEGFR2, for example the adnectin set out in SEQ ID NO:18.

Other examples of such antigen-binding proteins include TNFR2-Ig fusions linked to an epitope binding domain with a specificity for VEGF for example an anti-VEGF dAb or anti-VEGF anticalin, linked to the c-terminus or the n-terminus of the TNFR2-Ig fusion, for example a Receptor-Fc-epitope binding domain fusion comprising the TNFR2-Ig sequence set out in SEQ ID NO:34, which further comprises one or more epitope-binding domains which bind to VEGF, for example the dAb set out in SEQ ID NO:1, or the anticalin set out in SEQ ID NO:19.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987).

However, although we use the Kabat numbering convention for amino acid residues in variable domain sequences and full length antibody sequences throughout this specification, it will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Antigen binding proteins with CDR variants are also considered part of the invention. Such antigen-binding proteins may also have one or more further epitope binding domains with the same or different antigen-specificity attached to the c-terminus and/or the n-terminus of the heavy chain and/or the c-terminus and/or n-terminus of the light chain and/or the n-terminus or c-terminus of the receptor-Fc or receptor-Fc-dAb fusion.

In one embodiment of the present invention there is provided an antigen-binding protein according to the invention described herein and comprising a constant region such that the antibody or receptor-Fc fusion has reduced ADCC and/or complement activation or effector functionality. In one such embodiment the heavy chain constant region may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering, Kabat et al., (1983) “Sequences of Proteins of Immunological Interest”, US Dept. Health and Human Services).

In an embodiment, the Fc portion of the antigen binding protein is functionally disabled. Such Fc disablement may provide the antigen binding protein with an improved safety profile.

The invention also provides a method of reducing CDC function of antigen-binding proteins by positioning of the epitope binding domain on the heavy chain of the antibody, in particular, by positioning the epitope binding domain on the c-terminus of the heavy chain.

In one embodiment the antigen-binding proteins of the present invention will retain Fc functionality for example will be capable of one or both of ADCC and CDC activity.

The antigen-binding proteins of the invention may have some effector function. For example if the Immunoglobulin scaffold contains an Fc region derived from an antibody with effector function, for example if the Immunoglobulin scaffold comprises CH2 and CH3 from IgG1. Levels of effector function can be varied according to known techniques, for example by mutations in the CH2 domain, for example wherein the IgG1 CH2 domain has one or more mutations at positions selected from 239 and 332 and 330, for example the mutations are selected from S239D and I332E and A330L such that the antibody has enhanced effector function, and/or for example altering the glycosylation profile of the antigen-binding protein of the invention such that there is a reduction in fucosylation of the Fc region.

In one embodiment, the antigen-binding proteins comprise an epitope-binding domain which is a domain antibody (dAb), for example the epitope binding domain may be a human V_{H} or human V_{L}, or a camelid V_{HH} or a shark dAb (NARV).

In one embodiment the antigen-binding proteins comprise an epitope-binding domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which have been subjected to protein engineering in order to obtain binding to a ligand other than the natural ligand.

The antigen-binding proteins of the present invention may comprise a protein scaffold attached to an epitope binding domain which is an adnectin, for example an IgG scaffold with an adnectin attached to the c-terminus of the heavy chain, or it may comprise a protein scaffold attached to an adnectin, for example an IgG scaffold with an adnectin attached to the n-terminus of the heavy chain, or it may comprise a protein scaffold attached to an adnectin, for example an IgG scaffold with an adnectin attached to the c-terminus of the light chain, or it may comprise a protein scaffold attached to an adnectin, for example an IgG scaffold with an adnectin attached to the n-terminus of the light chain.

In other embodiments it may comprise a protein scaffold, for example an IgG scaffold, attached to an epitope binding domain which is a CTLA-4, for example an IgG scaffold with a CTLA-4 attached to the n-terminus of the heavy chain, or it may comprise for example an IgG scaffold with a CTLA-4 attached to the c-terminus of the heavy chain, or it may comprise for example an IgG scaffold with CTLA-4 attached to the n-terminus of the light chain, or it may comprise an IgG scaffold with CTLA-4 attached to the c-terminus of the light chain.

In other embodiments it may comprise a protein scaffold, for example an IgG scaffold, attached to an epitope binding domain which is a lipocalin, for example an IgG scaffold with a lipocalin attached to the n-terminus of the heavy chain, or it may comprise for example an IgG scaffold with a lipocalin attached to the c-terminus of the heavy chain, or it may comprise for example an IgG scaffold with a lipocalin attached to the n-terminus of the light chain, or it may comprise an IgG scaffold with a lipocalin attached to the c-terminus of the light chain.

In other embodiments it may comprise a protein scaffold, for example an IgG scaffold, attached to an epitope binding domain which is an SpA, for example an IgG scaffold with an SpA attached to the n-terminus of the heavy chain, or it may comprise for example an IgG scaffold with an SpA attached to the c-terminus of the heavy chain, or it may comprise for example an IgG scaffold with an SpA attached to the n-terminus of the light chain, or it may comprise an IgG scaffold with an SpA attached to the c-terminus of the light chain.

In other embodiments it may comprise a protein scaffold, for example an IgG scaffold, attached to an epitope binding domain which is an affibody, for example an IgG scaffold with an affibody attached to the n-terminus of the heavy chain, or it may comprise for example an IgG scaffold with an affibody attached to the c-terminus of the heavy chain, or it may comprise for example an IgG scaffold with an affibody attached to the n-terminus of the light chain, or it may comprise an IgG scaffold with an affibody attached to the c-terminus of the light chain.

In other embodiments it may comprise a protein scaffold, for example an IgG scaffold, attached to an epitope binding domain which is an affimer, for example an IgG scaffold with an affimer attached to the n-terminus of the heavy chain, or it may comprise for example an IgG scaffold with an affimer attached to the c-terminus of the heavy chain, or it may comprise for example an IgG scaffold with an affimer attached to the n-terminus of the light chain, or it may comprise an IgG scaffold with an affimer attached to the c-terminus of the light chain.

In other embodiments it may comprise a protein scaffold, for example an IgG scaffold, attached to an epitope binding domain which is a GroEI, for example an IgG scaffold with a GroEI attached to the n-terminus of the heavy chain, or it may comprise for example an IgG scaffold with a GroEI attached to the c-terminus of the heavy chain, or it may comprise for example an IgG scaffold with a GroEI attached to the n-terminus of the light chain, or it may comprise an IgG scaffold with a GroEI attached to the c-terminus of the light chain.

In other embodiments it may comprise a protein scaffold, for example an IgG scaffold, attached to an epitope binding domain which is a transferrin, for example an IgG scaffold with a transferrin attached to the n-terminus of the heavy chain, or it may comprise for example an IgG scaffold with a transferrin attached to the c-terminus of the heavy chain, or it may comprise for example an IgG scaffold with a transferrin attached to the n-terminus of the light chain, or it may comprise an IgG scaffold with a transferrin attached to the c-terminus of the light chain.

In other embodiments it may comprise a protein scaffold, for example an IgG scaffold, attached to an epitope binding domain which is a GroES, for example an IgG scaffold with a GroES attached to the n-terminus of the heavy chain, or it may comprise for example an IgG scaffold with a GroES attached to the c-terminus of the heavy chain, or it may comprise for example an IgG scaffold with a GroES attached to the n-terminus of the light chain, or it may comprise an IgG scaffold with a GroES attached to the c-terminus of the light chain.

In other embodiments it may comprise a protein scaffold, for example an IgG scaffold, attached to an epitope binding domain which is a DARPin, for example an IgG scaffold with a DARPin attached to the n-terminus of the heavy chain, or it may comprise for example an IgG scaffold with a DARPin attached to the c-terminus of the heavy chain, or it may comprise for example an IgG scaffold with a DARPin attached to the n-terminus of the light chain, or it may comprise an IgG scaffold with a DARPin attached to the c-terminus of the light chain.

In other embodiments it may comprise a protein scaffold, for example an IgG scaffold, attached to an epitope binding domain which is a peptide aptamer, for example an IgG scaffold with a peptide aptamer attached to the n-terminus of the heavy chain, or it may comprise for example an IgG scaffold with a peptide aptamer attached to the c-terminus of the heavy chain, or it may comprise for example an IgG scaffold with a peptide aptamer attached to the n-terminus of the light chain, or it may comprise an IgG scaffold with a peptide aptamer attached to the c-terminus of the light chain.

In one embodiment of the present invention there are four epitope binding domains, for example four domain antibodies, two of the epitope binding domains may have specificity for the same antigen, or all of the epitope binding domains present in the antigen-binding protein may have specificity for the same antigen.

Protein scaffolds of the present invention may be linked to epitope-binding domains by the use of linkers. Similarly receptor-Fc fusions of the present invention may be linked to epitope binding domains by the use of linkers. Also VD1 and VD2 domains of DVD-Igs may be linked together by means of linkers, and so forth. Examples of suitable linkers include amino acid sequences which may be from 1 amino acid to 150 amino acids in length, or from 1 amino acid to 140 amino acids, for example, from 1 amino acid to 130 amino acids, or from 1 to 120 amino acids, or from 1 to 80 amino acids, or from 1 to 50 amino acids, or from 1 to 20 amino acids, or from 1 to 10 amino acids, or from 5 to 18 amino acids. Such sequences may have their own tertiary structure, for example, a linker of the present invention may comprise a single variable domain. The size of a linker in one embodiment is equivalent to a single variable domain. Suitable linkers may be of a size from 1 to 20 angstroms, for example less than 15 angstroms, or less than 10 angstroms, or less than 5 angstroms.

In one embodiment of the present invention at least one of the epitope binding domains is directly attached to the Ig scaffold with a linker comprising from 1 to 150 amino acids, for example 1 to 20 amino acids, for example 1 to 10 amino acids.

Such linkers may be selected from any one of those set out in SEQ ID NO: 3-8, SEQ ID NO:25, or SEQ ID NO:66-68, or multiples of such linkers. For example, the linker may be ‘TVAAPS’, or the linker may be ‘GGGGS’, or multiples of such linkers.

In an embodiment of the invention the linker is ‘STG’ (SEQ ID NO:25).

A linker can be any linker as herein described with one or two amino acid changes. Linkers of use in the antigen-binding proteins of the present invention may comprise alone or in addition to other linkers, one or more sets of GS residues, for example ‘GSTVAAPS’ or ‘TVAAPSGS’ or ‘GSTVAAPSGS’, or multiples of such linkers. In an embodiment the linker comprises or consists of ‘GSTVAAPSGS’.

In an embodiment the linker comprises or consists of GS(TVAAPSGS)×2 (e.g. ‘GSTVAAPSGSTVAAPSGS’ SEQ ID NO:66). In an embodiment the linker comprises or consists of GS(TVAAPSGS)×3 (e.g. ‘GSTVAAPSGSTVAAPSGSTVAAPSGS’ SEQ ID NO:67). In an embodiment the linker comprises or consists of GS(TVAAPSGS)×4 (e.g. ‘GSTVAAPSGSTVAAPSGS TVAAPSGSTVAAPSGS’ SEQ ID NO:68).

In one embodiment the epitope binding domain is linked to the Ig scaffold by the linker ‘(PAS)ₙ(GS)ₘ’. In another embodiment the epitope binding domain is linked to the Ig scaffold by the linker ‘(GGGGS)_{n or p}(GS)ₘ’. In another embodiment the epitope binding domain is linked to the Ig scaffold by the linker ‘(TVAAPS)_{n or p}(GS)ₘ’. In another embodiment the epitope binding domain is linked to the Ig scaffold by the linker ‘(GS)ₘ(TVAAPSGS)_{n or p}’. In another embodiment the epitope binding domain is linked to the Ig scaffold by the linker ‘(GS)ₘ(TVAAPS)ₚ(GS)ₘ’. In another embodiment the epitope binding domain is linked to the Ig scaffold by the linker ‘(PAVPPP)ₙ(GS)ₘ’. In another embodiment the epitope binding domain is linked to the Ig scaffold by the linker ‘(TVSDVP)ₙ(GS)ₘ’. In another embodiment the epitope binding domain is linked to the Ig scaffold by the linker ‘(TGLDSP)ₙ(GS)ₘ’. In all such embodiments, n=1-10, and m=0-4, and p=2-10.

Examples of such linkers include (PAS)ₙ(GS)ₘ wherein n=1 and m=1 (SEQ ID NO:145), (PAS)ₙ(GS)ₘ wherein n=2 and m=1 (SEQ ID NO:146), (PAS)ₙ(GS)ₘ wherein n=3 and m=1 (SEQ ID NO:147), (PAS)ₙ(GS)ₘ wherein n=4 and m=1, (PAS)ₙ(GS)ₘ wherein n=2 and m=0, (PAS)ₙ(GS)ₘ wherein n=3 and m=0, (PAS)ₙ(GS)ₘ wherein n=4 and m=0.

Examples of such linkers include (GGGGS)ₙ(GS)ₘ wherein n=1 and m=1, (GGGGS)ₙ(GS)ₘ wherein n=2 and m=1, (GGGGS)ₙ(GS)ₘ wherein n=3 and m=1, (GGGGS)ₙ(GS)ₘ wherein n=4 and m=1, (GGGGS)ₙ(GS)ₘ wherein n=2 and m=0 (SEQ ID NO:148), (GGGGS)ₙ(GS)ₘ wherein n=3 and m=0 (SEQ ID NO:149), (GGGGS)ₙ(GS)ₘ wherein n=4 and m=0.

Examples of such linkers include (GS)ₘ(TVAAPS)ₚ wherein p=1 and m=1, (GS)ₘ(TVAAPS)ₚ wherein p=2 and m=1, (GS)ₘ(TVAAPS)ₚ wherein p=3 and m=1, (GS)ₘ(TVAAPS)ₚ wherein p=4 and m=1), (GS)ₘ(TVAAPS)ₚ wherein p=5 and m=1, or (GS)ₘ(TVAAPS)ₚ wherein p=6 and m=1.

Examples of such linkers include (TVAAPS)ₙ(GS)ₘ wherein n=1 and m=1, (TVAAPS)ₙ(GS)ₘ wherein n=2 and m=1 (SEQ ID NO:150), (TVAAPS)ₙ(GS)ₘ wherein n=3 and m=1 (SEQ ID NO:151), (TVAAPS)ₙ(GS)ₘ wherein n=4 and m=1, (TVAAPS)ₙ(GS)ₘ wherein n=2 and m=0, (TVAAPS)ₙ(GS)ₘ wherein n=3 and m=0, (TVAAPS)ₙ(GS)ₘ wherein n=4 and m=0.

Examples of such linkers include (GS)ₘ(TVAAPSGS)ₙ wherein n=1 and m=1, (GS)ₘ(TVAAPSGS)ₙ wherein n=2 and m=1 (SEQ ID NO:66), (GS)ₘ(TVAAPSGS)ₙ wherein n=3 and m=1 (SEQ ID NO:67), or (GS)ₘ(TVAAPSGS)ₙ wherein n=4 and m=1 (SEQ ID NO:68), (GS)ₘ(TVAAPSGS)ₙ wherein n=5 and m=1 (SEQ ID NO:152), (GS)ₘ(TVAAPSGS)ₙ wherein n=6 and m=1 (SEQ ID NO:153), (GS)ₘ(TVAAPSGS)ₙ wherein n=1 and m=0 (SEQ ID NO:8), (GS)ₘ(TVAAPSGS)ₙ wherein n=2 and m=10, (GS)ₘ(TVAAPSGS)ₙ wherein n=3 and m=0, or (GS)ₘ(TVAAPSGS)ₙ wherein n=0. Examples of such linkers include (TVAAPSGS)ₚ(GS)ₘ wherein p=2 and m=1, (TVAAPSGS)ₚ(GS)ₘ wherein p=3 and m=1, (TVAAPSGS)ₚ(GS)ₘ wherein p=4 and m=1, (TVAAPSGS)ₚ(GS)ₘ wherein p=2 and m=0, (TVAAPSGS)ₚ(GS)ₘ wherein p=3 and m=0, (TVAAPSGS)ₚ(GS)ₘ wherein p=4 and m=0.

Examples of such linkers include (PAVPPP)ₙ(GS)ₘ wherein n=1 and m=1 (SEQ ID NO:154), (PAVPPP)ₙ(GS)ₘ wherein n=2 and m=1 (SEQ ID NO:155), (PAVPPP)ₙ(GS)ₘ wherein n=3 and m=1 (SEQ ID NO:156), (PAVPPP)ₙ(GS)ₘ wherein n=4 and m=1, (PAVPPP)ₙ(GS)ₘ wherein n=2 and m=0, (PAVPPP)ₙ(GS)ₘ wherein n=3 and m=0, (PAVPPP)ₙ(GS)ₘ wherein n=4 and m=0.

Examples of such linkers include (TVSDVP)ₙ(GS)ₘ wherein n=1 and m=1 (SEQ ID NO:157), (TVSDVP)ₙ(GS)ₘ wherein n=2 and m=1 (SEQ ID NO:158), (TVSDVP)ₙ(GS)ₘ wherein n=3 and m=1 (SEQ ID NO:159), (TVSDVP)ₙ(GS)ₘ wherein n=4 and m=1, (TVSDVP)ₙ(GS)ₘ wherein n=2 and m=0, (TVSDVP)ₙ(GS)ₘ wherein n=3 and m=0, (TVSDVP)ₙ(GS)ₘ wherein n=4 and m=0.

Examples of such linkers include (TGLDSP)ₙ(GS)ₘ wherein n=1 and m=1 (SEQ ID NO:160), (TGLDSP)ₙ(GS)ₘ wherein n=2 and m=1 (SEQ ID NO:161), (TGLDSP)ₙ(GS)ₘ wherein n=3 and m=1 (SEQ ID NO:162), (TGLDSP)ₙ(GS)ₘ wherein n=4 and m=1, (TGLDSP)ₙ(GS)ₘ wherein n=2 and m=0, (TGLDSP)ₙ(GS)ₘ wherein n=3 and m=0, (TGLDSP)ₙ(GS)ₘ wherein n=4 and m=0.

In another embodiment there is no linker between the epitope binding domain and the Ig scaffold. In another embodiment the epitope binding domain is linked to the Ig scaffold by the linker ‘TVAAPS’. In another embodiment the epitope binding domain, is linked to the Ig scaffold by the linker ‘TVAAPSGS’. In another embodiment the epitope binding domain is linked to the Ig scaffold by the linker ‘GS’. In another embodiment the epitope binding domain is linked to the Ig scaffold by the linker ‘ASTKGPT’.

In one embodiment, the antigen-binding protein of the present invention comprises at least one antigen-binding site, for example at least one epitope binding domain, which is capable of binding human serum albumin.

In one embodiment, there are at least 3 antigen-binding sites, for example there are 4, or 5 or 6 or 8 or 10 antigen-binding sites and the antigen-binding protein is capable of binding at least 3 or 4 or 5 or 6 or 8 or 10 antigens, for example it is capable of binding 3 or 4 or 5 or 6 or 8 or 10 antigens simultaneously.

The invention also provides the antigen-binding proteins disclosed herein for use in medicine, for example for use in the manufacture of a medicament for treating a disease of the eye (alternatively referred to herein as an ‘eye disease’), for example diabetic macula edema (DME), cystoid macula edema, uveitis, AMD (Age related macular degeneration), choroidal neovascular AMD, geographic atrophy, diabetic retinopathy, retinal vein occlusion (BRVO and/or CRVO) and other maculopathies and ocular vasculopathies. In an embodiment, the disease to be treated is AMD. In another embodiment, the disease to be treated is DME.

The invention provides a method of treating a patient suffering from a disease of the eye, for example diabetic macula edema, cystoid macula edema, uveitis, AMD (Age related macular degeneration), choroidal neovascular AMD, geographic atrophy, diabetic retinopathy, retinal vein occlusion (BRVO and/or CRVO) and other maculopathies and ocular vasculopathies comprising administering a therapeutic amount of an antigen-binding protein of the invention.

The antigen-binding proteins of the invention may be used for the treatment of a disease of the eye, for example diabetic macula edema, cystoid macula edema, uveitis, AMD (Age related macular degeneration), choroidal neovascular AMD, geographic atrophy, diabetic retinopathy, retinal vein occlusion (BRVO and/or CRVO) and other maculopathies and ocular vasculopathies or any other disease associated with the over production of TNFα and/or VEGF.

In a particular embodiment the disease is AMD, specifically choroidal neovascular AMD.

Protein scaffolds of use in the present invention include full monoclonal antibody scaffolds comprising all the domains of an antibody, an Fc portion of a conventional antibody, or protein scaffolds of the present invention may comprise a non-conventional antibody structure, such as a monovalent antibody or an Fc portion of a non-conventional antibody structure. Such monovalent antibodies may comprise a paired heavy and light chain wherein the hinge region of the heavy chain is modified so that the heavy chain does not homodimerise, such as the monovalent antibody described in WO2007059782. Other monovalent antibodies may comprise a paired heavy and light chain which dimerises with a second heavy chain which is lacking a functional variable region and C_{H}1 region, wherein the first and second heavy chains are modified so that they will form heterodimers rather than homodimers, resulting in a monovalent antibody with two heavy chains and one light chain such as the monovalent antibody described in WO2006015371. Such monovalent antibodies can provide the protein scaffold of the present invention to which epitope binding domains can be linked. The Fc region of such monovalent antibodies can provide the Immunoglobulin scaffold of the present invention to which soluble ligands, extracellular domains of a receptor or cell surface protein and epitope binding domains can be linked. In such a monovalent structure it is possible to have a soluble ligand or extracellular domain of a receptor or cell surface protein linked to the first heavy chain and one or more epitope binding domains linked to the second heavy chain.

Epitope-binding domains of use in the present invention are domains that specifically bind an antigen or epitope independently of a different V region or domain, this may be a domain antibody or may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which have been subjected to protein engineering in order to obtain binding to a ligand other than the natural ligand. In one embodiment this may be an domain antibody or other suitable domains such as a domain selected from the group consisting of CTLA-4, lipocallin, SpA, an Affibody, an avimer, GroEI, transferrin, GroES and fibronectin. In one embodiment this may be selected from a dAb, an Affibody, an ankyrin repeat protein (DARPin) and an adnectin. In another embodiment this may be selected from an Affibody, an ankyrin repeat protein (DARPin) and an adnectin. In another embodiment this may be a domain antibody, for example a domain antibody selected from a human, camelid or shark (NARV) domain antibody.

Epitope-binding domains can be linked to the protein scaffold at one or more positions. These positions include the C-terminus and the N-terminus of the protein scaffold, for example at the C-terminus of the heavy chain and/or the C-terminus of the light chain of an IgG, or for example the N-terminus of the heavy chain and/or the N-terminus of the light chain of an IgG.

In one embodiment, a first epitope binding domain is linked to the protein scaffold and a second epitope binding domain is linked to the first epitope binding domain, for example where the protein scaffold is an IgG scaffold, a first epitope binding domain may be linked to the c-terminus of the heavy chain of the IgG scaffold, and that epitope binding domain can be linked at its c-terminus to a second epitope binding domain, or for example a first epitope binding domain may be linked to the c-terminus of the light chain of the IgG scaffold, and that first epitope binding domain may be further linked at its c-terminus to a second epitope binding domain, or for example a first epitope binding domain may be linked to the n-terminus of the light chain of the IgG scaffold, and that first epitope binding domain may be further linked at its n-terminus to a second epitope binding domain, or for example a first epitope binding domain may be linked to the n-terminus of the heavy chain of the IgG scaffold, and that first epitope binding domain may be further linked at its n-terminus to a second epitope binding domain.

When the epitope-binding domain is a domain antibody, some domain antibodies may be suited to particular positions within the scaffold.

Domain antibodies of use in the present invention can be linked at the C-terminal end of the heavy chain and/or the light chain of conventional IgGs. In addition some dAbs can be linked to the C-terminal ends of both the heavy chain and the light chain of conventional antibodies.

Epitope-binding domains can be linked to the Receptor-Fc fusion at one or more positions. These positions include the C-terminus and the N-terminus of the Receptor-Fc fusion. For example they may be linked directly to the Fc portion of the Receptor-Fc fusion, or they may be linked to the soluble ligand or extracellular domain of a receptor or cell surface protein portion of the Receptor-Fc fusion. Where the soluble ligand or extracellular domain of a receptor or cell surface protein is linked to the N-terminus of the Fc portion, the epitope-binding domain may be linked directly to the c-terminus of the Fc portion or to the N-terminus of the soluble ligand or extracellular domain of a receptor or cell surface protein.

In one embodiment, a first epitope binding domain is linked to the Receptor-Fc fusion and a second epitope binding domain is linked to the first epitope binding domain, for example a first epitope binding domain may be linked to the c-terminus of the Receptor-Fc fusion, and that epitope binding domain can be linked at its c-terminus to a second epitope binding domain, or for example a first epitope binding domain may be linked to the n-terminus of the Receptor-Fc fusion, and that first epitope binding domain may be further linked at its n-terminus to a second epitope binding domain, When the epitope-binding domain is a domain antibody, some domain antibodies may be suited to particular positions within the scaffold.

In constructs where the N-terminus of dAbs are fused to an antibody constant domain (either C_{H}3 or C_{L}), a peptide linker may help the dAb to bind to antigen. Indeed, the N-terminal end of a dAb is located closely to the complementarity-determining regions (CDRS) involved in antigen-binding activity. Thus a short peptide linker acts as a spacer between the epitope-binding, and the constant domain of the protein scaffold, which may allow the dAb CDRs to more easily reach the antigen, which may therefore bind with high affinity.

The surroundings in which dAbs are linked to the IgG will differ depending on which antibody chain they are fused to. When fused at the C-terminal end of the antibody light chain of an IgG scaffold, each dAb is expected to be located in the vicinity of the antibody hinge and the Fc portion. It is likely that such dAbs will be located far apart from each other. In conventional antibodies, the angle between Fab fragments and the angle between each Fab fragment and the Fc portion can vary quite significantly. It is likely that—with mAbdAbs—the angle between the Fab fragments will not be widely different, whilst some angular restrictions may be observed with the angle between each Fab fragment and the Fc portion.

When fused at the C-terminal end of the antibody heavy chain of an IgG scaffold, each dAb is expected to be located in the vicinity of the C_{H}3 domains of the Fc portion. This is not expected to impact on the Fc binding properties to Fc receptors (e.g. FcγRI, II, III an FcRn) as these receptors engage with the C_{H}2 domains (for the FcγRI, II and III class of receptors) or with the hinge between the C_{H}2 and C_{H}3 domains (e.g. FcRn receptor). Another feature of such antigen-binding proteins is that both dAbs are expected to be spatially close to each other and provided that flexibility is provided by provision of appropriate linkers, these dAbs may even form homodimeric species, hence propagating the ‘zipped’ quaternary structure of the Fc portion, which may enhance stability of the construct.

Such structural considerations can aid in the choice of the most suitable position to link an epitope-binding domain, for example a dAb, on to a protein scaffold, for example an antibody or on to a Receptor-Fc fusion.

The size of the antigen, its localization (in blood or on a cell surface), its quaternary structure (monomeric or multimeric) can vary. Conventional antibodies are naturally designed to function as adaptor constructs due to the presence of the hinge region, wherein the orientation of the two antigen-binding sites at the tip of the Fab fragments can vary widely and hence adapt to the molecular feature of the antigen and its surroundings. In contrast dAbs linked to an antibody or other protein scaffold, for example a protein scaffold which comprises an antibody with no hinge region, may have less structural flexibility either directly or indirectly.

Understanding the solution state and mode of binding at the dAb is also helpful. Evidence has accumulated that in vitro dAbs can predominantly exist in monomeric, homo-dimeric or multimeric forms in solution (Reiter et al., J Mol Biol (1999) 290: 685-698; Ewert et al., J Mol Biol (2003) 325: 531-553, Jespers et al., J Mol Biol (2004) 337: 893-903; Jespers et al., Nat Biotechnol (2004) 22: 1161-1165; Martin et al., Protein Eng. (1997) 10: 607-614; Sepulvada et al., J Mol Biol (2003) 333: 355-365). This is fairly reminiscent to multimerisation events observed in vivo with Ig domains such as Bence-Jones proteins (which are dimers of immunoglobulin light chains (Epp et al., Biochemistry (1975) 14: 4943-4952; Huan et al., Biochemistry (1994) 33: 14848-14857; Huang et al., Mol immunol (1997) 34: 1291-1301) and amyloid fibers (James et al. J Mol. Biol. (2007) 367: 603-8).

For example, it may be desirable to link domain antibodies that tend to dimerise in solution to the C-terminal end of the Fc portion in preference to the C-terminal end of the light chain or the N-terminal end of the Receptor-Fc fusion as linking to the C-terminal end of the Fc will allow those dAbs to dimerise in the context of the antigen-binding protein of the invention.

The antigen-binding proteins of the present invention may comprise antigen-binding sites specific for a single antigen, or may have antigen-binding sites specific for two or more antigens, or for two or more epitopes on a single antigen, or there may be antigen-binding sites each of which is specific for a different epitope on the same or different antigens.

In particular, the antigen-binding proteins of the present invention may be useful in treating diseases associated with TNFα and VEGF for example diseases of the eye, for example diabetic macula edema, cystoid macula edema, uveitis, AMD (Age related macular degeneration), choroidal neovascular AMD, geographic atrophy, diabetic retinopathy, retinal vein occlusion (BRVO and/or CRVO) and other maculopathies and ocular vasculopathies.

Particular TNFα antagonists and VEGF antagonists which may be administered in combination for the treatment of any of the aforementioned diseases of the eye, in particular AMD, are as follows.

In an embodiment, the TNFα antagonist is adalimumab and the VEGF antagonist is bevacizumab. In an embodiment, the TNFα antagonist is adalimumab and the VEGF antagonist is ranibizumab. In an embodiment, the TNFα antagonist is adalimumab and the VEGF antagonist is r84. In an embodiment, the TNFα antagonist is adalimumab and the VEGF antagonist is aflibercept. In an embodiment, the TNFα antagonist is adalimumab and the VEGF antagonist is CT01. In an embodiment, the TNFα antagonist is adalimumab and the VEGF antagonist is DOM15-10-11. In an embodiment, the TNFα antagonist is adalimumab and the VEGF antagonist is DOM15-26-593. In an embodiment, the TNFα antagonist is adalimumab and the VEGF antagonist is PRS-050. In an embodiment, the TNFα antagonist is adalimumab and the VEGF antagonist is PRS-051. In an embodiment, the TNFα antagonist is adalimumab and the VEGF antagonist is MP0112. In an embodiment, the TNFα antagonist is adalimumab and the VEGF antagonist is CT-322. In an embodiment, the TNFα antagonist is adalimumab and the VEGF antagonist is ESBA903. In an embodiment, the TNFα antagonist is adalimumab and the VEGF antagonist is EPI-0030. In an embodiment, the TNFα antagonist is adalimumab and the VEGF antagonist is EPI-0010. In an embodiment, the TNFα antagonist is adalimumab and the VEGF antagonist is DMS1571.

In an embodiment, the TNFα antagonist is infliximab and the VEGF antagonist is bevacizumab. In an embodiment, the TNFα antagonist is infliximab and the VEGF antagonist is ranibizumab. In an embodiment, the TNFα antagonist is infliximab and the VEGF antagonist is r84. In an embodiment, the TNFα antagonist is infliximab and the VEGF antagonist is aflibercept. In an embodiment, the TNFα antagonist is infliximab and the VEGF antagonist is CT01. In an embodiment, the TNFα antagonist is infliximab and the VEGF antagonist is DOM15-10-11. In an embodiment, the TNFα antagonist is infliximab and the VEGF antagonist is DOM15-26-593. In an embodiment, the TNFα antagonist is infliximab and the VEGF antagonist is PRS-050. In an embodiment, the TNFα antagonist is infliximab and the VEGF antagonist is PRS-051. In an embodiment, the TNFα antagonist is infliximab and the VEGF antagonist is MP0112. In an embodiment, the TNFα antagonist is infliximab and the VEGF antagonist is CT-322. In an embodiment, the TNFα antagonist is infliximab and the VEGF antagonist is ESBA903. In an embodiment, the TNFα antagonist is infliximab and the VEGF antagonist is EPI-0030. In an embodiment, the TNFα antagonist is infliximab and the VEGF antagonist is EPI-0010. In an embodiment, the TNFα antagonist is infliximab and the VEGF antagonist is DMS1571.

In an embodiment, the TNFα antagonist is etanercept and the VEGF antagonist is bevacizumab. In an embodiment, the TNFα antagonist is etanercept and the VEGF antagonist is ranibizumab. In an embodiment, the TNFα antagonist is etanercept and the VEGF antagonist is r84. In an embodiment, the TNFα antagonist is etanercept and the VEGF antagonist is aflibercept. In an embodiment, the TNFα antagonist is etanercept and the VEGF antagonist is CT01. In an embodiment, the TNFα antagonist is etanercept and the VEGF antagonist is DOM15-10-11. In an embodiment, the TNFα antagonist is etanercept and the VEGF antagonist is DOM15-26-593. In an embodiment, the TNFα antagonist is etanercept and the VEGF antagonist is PRS-050. In an embodiment, the TNFα antagonist is etanercept and the VEGF antagonist is PRS-051. In an embodiment, the TNFα antagonist is etanercept and the VEGF antagonist is MP0112. In an embodiment, the TNFα antagonist is etanercept and the VEGF antagonist is CT-322. In an embodiment, the TNFα antagonist is etanercept and the VEGF antagonist is ESBA903. In an embodiment, the TNFα antagonist is etanercept and the VEGF antagonist is EPI-0030. In an embodiment, the TNFα antagonist is etanercept and the VEGF antagonist is EPI-0010. In an embodiment, the TNFα antagonist is etanercept and the VEGF antagonist is DMS1571.

In an embodiment, the TNFα antagonist is ESBA105 and the VEGF antagonist is bevacizumab. In an embodiment, the TNFα antagonist is ESBA105 and the VEGF antagonist is ranibizumab. In an embodiment, the TNFα antagonist is ESBA105 and the VEGF antagonist is r84. In an embodiment, the TNFα antagonist is ESBA105 and the VEGF antagonist is aflibercept. In an embodiment, the TNFα antagonist is ESBA105 and the VEGF antagonist is CT01. In an embodiment, the TNFα antagonist is ESBA105 and the VEGF antagonist is DOM15-10-11. In an embodiment, the TNFα antagonist is ESBA105 and the VEGF antagonist is DOM15-26-593. In an embodiment, the TNFα antagonist is ESBA105 and the VEGF antagonist is PRS-050. In an embodiment, the TNFα antagonist is ESBA105 and the VEGF antagonist is PRS-051. In an embodiment, the TNFα antagonist is ESBA105 and the VEGF antagonist is MP0112. In an embodiment, the TNFα antagonist is ESBA105 and the VEGF antagonist is CT-322. In an embodiment, the TNFα antagonist is ESBA105 and the VEGF antagonist is ESBA903. In an embodiment, the TNFα antagonist is ESBA105 and the VEGF antagonist is EPI-0030. In an embodiment, the TNFα antagonist is ESBA105 and the VEGF antagonist is EPI-0010. In an embodiment, the TNFα antagonist is ESBA105 and the VEGF antagonist is DMS1571.

In an embodiment, the TNFα antagonist is PEP1-5-19 and the VEGF antagonist is bevacizumab. In an embodiment, the TNFα antagonist is PEP1-5-19 and the VEGF antagonist is ranibizumab. In an embodiment, the TNFα antagonist is PEP1-5-19 and the VEGF antagonist is r84. In an embodiment, the TNFα antagonist is PEP1-5-19 and the VEGF antagonist is aflibercept. In an embodiment, the TNFα antagonist is PEP1-5-19 and the VEGF antagonist is CT01. In an embodiment, the TNFα antagonist is PEP1-5-19 and the VEGF antagonist is DOM15-10-11. In an embodiment, the TNFα antagonist is PEP1-5-19 and the VEGF antagonist is DOM15-26-593. In an embodiment, the TNFα antagonist is PEP1-5-19 and the VEGF antagonist is PRS-050. In an embodiment, the TNFα antagonist is PEP1-5-19 and the VEGF antagonist is PRS-051. In an embodiment, the TNFα antagonist is PEP1-5-19 and the VEGF antagonist is MP0112. In an embodiment, the TNFα antagonist is PEP1-5-19 and the VEGF antagonist is CT-322. In an embodiment, the TNFα antagonist is PEP1-5-19 and the VEGF antagonist is ESBA903. In an embodiment, the TNFα antagonist is PEP1-5-19 and the VEGF antagonist is EPI-0030. In an embodiment, the TNFα antagonist is PEP1-5-19 and the VEGF antagonist is EPI-0010. In an embodiment, the TNFα antagonist is PEP1-5-19 and the VEGF antagonist is DMS1571.

In an embodiment, the TNFα antagonist is PEP1-5-490 and the VEGF antagonist is bevacizumab. In an embodiment, the TNFα antagonist is PEP1-5-490 and the VEGF antagonist is ranibizumab. In an embodiment, the TNFα antagonist is PEP1-5-490 and the VEGF antagonist is r84. In an embodiment, the TNFα antagonist is PEP1-5-490 and the VEGF antagonist is aflibercept. In an embodiment, the TNFα antagonist is PEP1-5-490 and the VEGF antagonist is CT01. In an embodiment, the TNFα antagonist is PEP1-5-490 and the VEGF antagonist is DOM15-10-11. In an embodiment, the TNFα antagonist is PEP1-5-490 and the VEGF antagonist is DOM15-26-593. In an embodiment, the TNFα antagonist is PEP1-5-490 and the VEGF antagonist is PRS-050. In an embodiment, the TNFα antagonist is PEP1-5-490 and the VEGF antagonist is PRS-051. In an embodiment, the TNFα antagonist is PEP1-5-490 and the VEGF antagonist is MP0112. In an embodiment, the TNFα antagonist is PEP1-5-490 and the VEGF antagonist is CT-322. In an embodiment, the TNFα antagonist is PEP1-5-490 and the VEGF antagonist is ESBA903. In an embodiment, the TNFα antagonist is PEP1-5-490 and the VEGF antagonist is EPI-0030. In an embodiment, the TNFα antagonist is PEP1-5-490 and the VEGF antagonist is EPI-0010. In an embodiment, the TNFα antagonist is PEP1-5-490 and the VEGF antagonist is DMS1571.

In an embodiment, the TNFα antagonist is PEP1-5-493 and the VEGF antagonist is bevacizumab. In an embodiment, the TNFα antagonist is PEP1-5-493 and the VEGF antagonist is ranibizumab. In an embodiment, the TNFα antagonist is PEP1-5-493 and the VEGF antagonist is r84. In an embodiment, the TNFα antagonist is PEP1-5-493 and the VEGF antagonist is aflibercept. In an embodiment, the TNFα antagonist is PEP1-5-493 and the VEGF antagonist is CT01. In an embodiment, the TNFα antagonist is PEP1-5-493 and the VEGF antagonist is DOM15-10-11. In an embodiment, the TNFα antagonist is PEP1-5-493 and the VEGF antagonist is DOM15-26-593. In an embodiment, the TNFα antagonist is PEP1-5-493 and the VEGF antagonist is PRS-050. In an embodiment, the TNFα antagonist is PEP1-5-493 and the VEGF antagonist is PRS-051. In an embodiment, the TNFα antagonist is PEP1-5-493 and the VEGF antagonist is MP0112. In an embodiment, the TNFα antagonist is PEP1-5-493 and the VEGF antagonist is CT-322. In an embodiment, the TNFα antagonist is PEP1-5-493 and the VEGF antagonist is ESBA903. In an embodiment, the TNFα antagonist is PEP1-5-493 and the VEGF antagonist is EPI-0030. In an embodiment, the TNFα antagonist is PEP1-5-493 and the VEGF antagonist is EPI-0010. In an embodiment, the TNFα antagonist is PEP1-5-493 and the VEGF antagonist is DMS1571.

In an embodiment, the TNFα antagonist is the adnectin of SEQ ID NO:2 and the VEGF antagonist is bevacizumab. In an embodiment, the TNFα antagonist is the adnectin of SEQ ID NO:2 and the VEGF antagonist is ranibizumab. In an embodiment, the TNFα antagonist is the adnectin of SEQ ID NO:2 and the VEGF antagonist is r84. In an embodiment, the TNFα antagonist is the adnectin of SEQ ID NO:2 and the VEGF antagonist is aflibercept. In an embodiment, the TNFα antagonist is the adnectin of SEQ ID NO:2 and the VEGF antagonist is CT01. In an embodiment, the TNFα antagonist is the adnectin of SEQ ID NO:2 and the VEGF antagonist is DOM15-10-11. In an embodiment, the TNFα antagonist is the adnectin of SEQ ID NO:2 and the VEGF antagonist is DOM15-26-593. In an embodiment, the TNFα antagonist is the adnectin of SEQ ID NO:2 and the VEGF antagonist is PRS-050. In an embodiment, the TNFα antagonist is the adnectin of SEQ ID NO:2 and the VEGF antagonist is PRS-051. In an embodiment, the TNFα antagonist is the adnectin of SEQ ID NO:2 and the VEGF antagonist is MP0112. In an embodiment, the TNFα antagonist is the adnectin of SEQ ID NO:2 and the VEGF antagonist is CT-322. In an embodiment, the TNFα antagonist is the adnectin of SEQ ID NO:2 and the VEGF antagonist is ESBA903. In an embodiment, the TNFα antagonist is the adnectin of SEQ ID NO:2 and the VEGF antagonist is EPI-0030. In an embodiment, the TNFα antagonist is the adnectin of SEQ ID NO:2 and the VEGF antagonist is EPI-0010. In an embodiment, the TNFα antagonist is the adnectin of SEQ ID NO:2 and the VEGF antagonist is DMS1571

In an embodiment, the TNFα antagonist is golimumab and the VEGF antagonist is bevacizumab. In an embodiment, the TNFα antagonist is golimumab and the VEGF antagonist is ranibizumab. In an embodiment, the TNFα antagonist is golimumab and the VEGF antagonist is r84. In an embodiment, the TNFα antagonist is golimumab and the VEGF antagonist is aflibercept. In an embodiment, the TNFα antagonist is golimumab and the VEGF antagonist is CT01. In an embodiment, the TNFα antagonist is golimumab and the VEGF antagonist is DOM15-10-11. In an embodiment, the TNFα antagonist is golimumab and the VEGF antagonist is DOM15-26-593. In an embodiment, the TNFα antagonist is golimumab and the VEGF antagonist is PRS-050. In an embodiment, the TNFα antagonist is golimumab and the VEGF antagonist is PRS-051. In an embodiment, the TNFα antagonist is golimumab and the VEGF antagonist is MP0112. In an embodiment, the TNFα antagonist is golimumab and the VEGF antagonist is CT-322. In an embodiment, the TNFα antagonist is golimumab and the VEGF antagonist is ESBA903. In an embodiment, the TNFα antagonist is golimumab and the VEGF antagonist is EPI-0030. In an embodiment, the TNFα antagonist is golimumab and the VEGF antagonist is EPI-0010. In an embodiment, the TNFα antagonist is golimumab and the VEGF antagonist is DMS1571.

In an embodiment, the TNFα antagonist is certolizumab and the VEGF antagonist is bevacizumab. In an embodiment, the TNFα antagonist is certolizumab and the VEGF antagonist is ranibizumab. In an embodiment, the TNFα antagonist is certolizumab and the VEGF antagonist is r84. In an embodiment, the TNFα antagonist is certolizumab and the VEGF antagonist is aflibercept. In an embodiment, the TNFα antagonist is certolizumab and the VEGF antagonist is CT01. In an embodiment, the TNFα antagonist is certolizumab and the VEGF antagonist is DOM15-10-11. In an embodiment, the TNFα antagonist is certolizumab and the VEGF antagonist is DOM15-26-593. In an embodiment, the TNFα antagonist is certolizumab and the VEGF antagonist is PRS-050. In an embodiment, the TNFα antagonist is certolizumab and the VEGF antagonist is PRS-051. In an embodiment, the TNFα antagonist is certolizumab and the VEGF antagonist is MP0112. In an embodiment, the TNFα antagonist is certolizumab and the VEGF antagonist is CT-322. In an embodiment, the TNFα antagonist is certolizumab and the VEGF antagonist is ESBA903. In an embodiment, the TNFα antagonist is certolizumab and the VEGF antagonist is EPI-0030. In an embodiment, the TNFα antagonist is certolizumab and the VEGF antagonist is EPI-0010. In an embodiment, the TNFα antagonist is certolizumab and the VEGF antagonist is DMS1571.

In an embodiment, the TNFα antagonist is ALK-6931 and the VEGF antagonist is bevacizumab. In an embodiment, the TNFα antagonist is ALK-6931 and the VEGF antagonist is ranibizumab. In an embodiment, the TNFα antagonist is ALK-6931 and the VEGF antagonist is r84. In an embodiment, the TNFα antagonist is ALK-6931 and the VEGF antagonist is aflibercept. In an embodiment, the TNFα antagonist is ALK-6931 and the VEGF antagonist is CT01. In an embodiment, the TNFα antagonist is ALK-6931 and the VEGF antagonist is DOM15-10-11. In an embodiment, the TNFα antagonist is ALK-6931 and the VEGF antagonist is DOM15-26-593. In an embodiment, the TNFα antagonist is ALK-6931 and the VEGF antagonist is PRS-050. In an embodiment, the TNFα antagonist is ALK-6931 and the VEGF antagonist is PRS-051. In an embodiment, the TNFα antagonist is ALK-6931 and the VEGF antagonist is MP0112. In an embodiment, the TNFα antagonist is ALK-6931 and the VEGF antagonist is CT-322. In an embodiment, the TNFα antagonist is ALK-6931 and the VEGF antagonist is ESBA903. In an embodiment, the TNFα antagonist is ALK-6931 and the VEGF antagonist is EPI-0030. In an embodiment, the TNFα antagonist is ALK-6931 and the VEGF antagonist is EPI-0010. In an embodiment, the TNFα antagonist is ALK-6931 and the VEGF antagonist is DMS1571.

In an embodiment, the TNFα antagonist is an antibody comprising a heavy chain of SEQ ID NO:30 and a light chain or SEQ ID NO:31 and the VEGF antagonist is bevacizumab. In an embodiment, the TNFα antagonist is an antibody comprising a heavy chain of SEQ ID NO:30 and a light chain or SEQ ID NO:31 and the VEGF antagonist is ranibizumab. In an embodiment, the TNFα antagonist is an antibody comprising a heavy chain of SEQ ID NO:30 and a light chain of SEQ ID NO:31 and the VEGF antagonist is r84. In an embodiment, the TNFα antagonist is an antibody comprising a heavy chain of SEQ ID NO:30 and a light chain of SEQ ID NO:31 and the VEGF antagonist is aflibercept. In an embodiment, the TNFα antagonist is an antibody comprising a heavy chain of SEQ ID NO:30 and a light chain of SEQ ID NO:31 and the VEGF antagonist is CT01. In an embodiment, the TNFα antagonist is an antibody comprising a heavy chain of SEQ ID NO:30 and a light chain of SEQ ID NO:31 and the VEGF antagonist is DOM15-10-11. In an embodiment, the TNFα antagonist is an antibody comprising a heavy chain of SEQ ID NO:30 and a light chain of SEQ ID NO:31 and the VEGF antagonist is DOM15-26-593. In an embodiment, the TNFα antagonist is an antibody comprising a heavy chain of SEQ ID NO:30 and a light chain of SEQ ID NO:31 and the VEGF antagonist is PRS-050. In an embodiment, the TNFα antagonist is an antibody comprising a heavy chain of SEQ ID NO:30 and a light chain of SEQ ID NO:31 and the VEGF antagonist is PRS-051. In an embodiment, the TNFα antagonist is an antibody comprising a heavy chain of SEQ ID NO:30 and a light chain of SEQ ID NO:31 and the VEGF antagonist is MP0112. In an embodiment, the TNFα antagonist is an antibody comprising a heavy chain of SEQ ID NO:30 and a light chain of SEQ ID NO:31 and the VEGF antagonist is CT-322. In an embodiment, the TNFα antagonist is an antibody comprising a heavy chain of SEQ ID NO:30 and a light chain of SEQ ID NO:31 and the VEGF antagonist is ESBA903. In an embodiment, the TNFα antagonist is an antibody comprising a heavy chain of SEQ ID NO:30 and a light chain of SEQ ID NO:31 and the VEGF antagonist is EPI-0030. In an embodiment, the TNFα antagonist is an antibody comprising a heavy chain of SEQ ID NO:30 and a light chain of SEQ ID NO:31 and the VEGF antagonist is EPI-0010. In an embodiment, the TNFα antagonist is an antibody comprising a heavy chain of SEQ ID NO:30 and a light chain of SEQ ID NO:31 and the VEGF antagonist is DMS1571.

Each of the above combinations may also be used to generate dual targeting molecules of the invention. Particular and non-limiting examples of dual targeting molecules of the invention are as follows: Fc enabled DMS4000 (SEQ ID NO:14 and SEQ ID NO:12), Fc disabled DMS4000 (SEQ ID NO:47 and SEQ ID NO: 12), DMS4031 (SEQ ID NO: 16 and SEQ ID NO:12), DOM-PEP in-line fusion (SEQ ID NO:62), PEP-DOM in-line fusion (SEQ ID NO: 64), a dual targeting molecule having a heavy chain selected from SEQ ID NO:69-72 and a light chain of SEQ ID NO:12, and those listed in SEQ ID NO:72-140.

The antigen-binding proteins of the present invention may be produced by transfection of a host cell with an expression vector comprising the coding sequence for the antigen-binding protein of the invention. An expression vector or recombinant plasmid is produced by placing these coding sequences for the antigen-binding protein in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antigen-binding protein light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antigen-binding protein may reside on a single vector, for example in two expression cassettes in the same vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antigen-binding protein of the invention. The antigen-binding protein which includes the association of both the recombinant heavy chain and/or light chain is screened from culture by appropriate assay, such as ELISA or RIA. Similar conventional techniques may be employed to construct other antigen-binding proteins.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham (Buckinghamshire, United Kingdom) or Pharmacia (Uppsala, Sweden). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antigen-binding proteins of the present invention. Host cells useful for the cloning and other manipulations of these cloning vectors are also conventional. However, cells from various strains of E. coli may be used for replication of the cloning vectors and other steps in the construction of antigen-binding proteins of this invention.

Suitable host cells or cell lines for the expression of the antigen-binding proteins of the invention include mammalian cells such as NSO, Sp2/0, CHO (e.g. DG44), COS, HEK, a fibroblast cell (e.g., 3T3), and myeloma cells, for example they may be expressed in a CHO or a myeloma cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., cited above.

Bacterial cells may prove useful as host cells suitable for the expression of the recombinant Fabs or other embodiments of the present invention (see, e.g., Plückthun, A., Immunol. Rev. (1992) 130: 151-188). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering (1986) δ: 277-298, Plenum Press and references cited therein.

The general methods by which the vectors may be constructed, the transfection methods required to produce the host cells of the invention, and culture methods necessary to produce the antigen-binding protein of the invention from such host cell may all be conventional techniques. Typically, the culture method of the present invention is a serum-free culture method, usually by culturing cells serum-free in suspension. Likewise, once produced, the antigen-binding proteins of the invention may be purified from the cell culture contents according to standard procedures of the art, including ammonium sulfate precipitation, affinity columns, column chromatography, gel electrophoresis and the like. Such techniques are within the skill of the art and do not limit this invention. For example, preparation of altered antibodies are described in WO 99/58679 and WO 96/16990.

Yet another method of expression of the antigen-binding proteins may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

In a further aspect of the invention there is provided a method of producing an antibody of the invention which method comprises the step of culturing a host cell transformed or transfected with a vector encoding the light and/or heavy chain of the antibody of the invention and recovering the antibody thereby produced.

In accordance with the present invention there is provided a method of producing an antigen-binding protein of the present invention which method comprises the steps of;
(a) providing a vector comprising a polynucleotide encoding the antigen-binding protein
(b) transforming a mammalian host cell (e.g. CHO) with said vector;
(c) culturing the host cell of step (b) under conditions conducive to the secretion of the antigen-binding protein from said host cell into said culture media;
(d) recovering the secreted antigen-binding protein of step (c).

In accordance with the present invention there is provided a method of producing an antigen-binding protein of the present invention which method comprises the steps of;
(a) providing a first vector encoding a heavy chain of the antigen-binding protein;
(b) providing a second vector encoding a light chain of the antigen-binding protein;
(c) transforming a mammalian host cell (e.g. CHO) with said first and second vectors;
(d) culturing the host cell of step (c) under conditions conducive to the secretion of the antigen-binding protein from said host cell into said culture media;
(e) recovering the secreted antigen-binding protein of step (d).

Once expressed by the desired method, the antigen-binding protein is then examined for in vitro activity by use of an appropriate assay. Presently conventional ELISA assay formats are employed to assess qualitative and quantitative binding of the antigen-binding protein to its target. Additionally, other in vitro assays may also be used to verify neutralizing efficacy prior to subsequent human clinical studies performed to evaluate the persistence of the antigen-binding protein in the body despite the usual clearance mechanisms.

The dose and duration of treatment relates to the relative duration of the molecules of the present invention in the human circulation, and can be adjusted by one of skill in the art depending upon the condition being treated and the general health of the patient. It is envisaged that repeated dosing (e.g. once a week or once every two weeks) over an extended time period (e.g. four to six months) maybe required to achieve maximal therapeutic efficacy.

The mode of administration of the therapeutic agent of the invention may be any suitable route which delivers the agent to the eye of the host. Systemic administration may be sufficient to deliver effective amounts of the antigen-binding proteins and pharmaceutical compositions of the invention via passive, e.g. intravenous or subcutaneous, administration. The antigen-binding proteins and pharmaceutical compositions of the invention may also be delivered more locally to the eye either by topical application e.g. eye drops or a gel, intravitreal injection, intracameral or periocular administration, i.e. subsclerally via either retrobulbar, peribulbar, subtenon or subconjunctival injection or via delivery to the inferior, superior or lateral rectus muscle. Other routes of local administration may allow the antigen-binding proteins and pharmaceutical compositions of the invention to reach the posterior segment of the eye more readily at lower doses. Topical application has been described to allow penetrance of antibody fragments to the posterior of the eye in the rabbit model, (Williams K A et al., (2005)). Intravitreal injection of antibody fragments or full monoclonal antibodies has been described and is well-tolerated for AMD patients for the products ranibizumab and bevacizumab.

In an embodiment, the TNF antagonist and the VEGF antagonist are both administered intravitreally. In an embodiment, the VEGF antagonist is administered intravitreally and the TNF antagonist, in particular ESBA105, is administered by a means other than topically e.g. also intravitreally or subconjunctivally. In an embodiment the TNF antagonist is administered intravitreally and the VEGF antagonist is administered topically.

It can be useful to target the delivery of the antigen binding protein into particular regions of the eye such as the surface of the eye, or to the tear ducts or lachrymal glands or there can be intra-ocular delivery (e.g. to the anterior or posterior chambers of the eye, such as the vitreous humour) and to ocular structures such as the iris, ciliary body, lachrymal gland. Hence the invention further provides a method of delivering a composition directly to the eye which comprises administering said composition to the eye by a method selected from: intra-ocular injection, topical delivery (e.g. eye drops), pen-ocular administration and use of a slow release formulation.

It can also be useful if the antigen binding protein is delivered to the eye e.g. by topical delivery (e.g. as eye drops), along with an ocular penetration enhancer e.g. sodium caprate, or with a viscosity enhancer e.g. Hydroxypropylmethylcellulose (HPMC). Accordingly the invention further provides compositions comprising (a) antigen binding protein of the invention and also (b) an ocular penetration enhancer and/or (c) a viscosity enhancer e.g. for topical delivery to the eye.

Delivery of the antigen-binding proteins and pharmaceutical compositions of the invention may also be administered by an intravitreal implant. Retrobulbar and peribulbar injections can be achieved with special 23 to 26 gauge needles and are less invasive than intravitreal injections. Subtenon injection places the composition in contact with the sclera for a longer period which could aid penetration to the posterior eye. Injection of proteins just beneath the conjuctiva has been described in rabbit models and this allows molecules to diffuse more directly across the sclera to reach the posterior segment of the eye.

Sustained release drug delivery systems may also be used which allow for release of material over a longer time-frame into or around the eye so that dosing could be less frequent. Such systems include micelles, gels, hydrogels, nanoparticles, microcapsules or implants that can be filled or coated with therapeutic compositions. These may be delivered into the vitreous of the eye by injection or by any of the other previously described less invasive routes, i.e. through the periocular or sub-scleral routes. Examples of such sustained release systems and local delivery routes include thermo-sensitive slow release hydrogels for subscleral administration or intravitreal administration of a nanoparticle based formulation that targets to the posterior retina and RPE layer (Janoira K G, et al., (2007); Birch D G (2007)). Many other combinations of delivery system and local administration route are possible and could be considered for compositions of the antigen-binding proteins, and pharmaceutical compositions of the invention.

In a particular embodiment, an antigen binding protein of the invention is administered intravitreally by intravitreal injection. In a particular embodiment, an antigen protein of the invention, in particular a dual targeting construct, is administered intravitreally every 4-8 weeks, preferably every 6-8 weeks. In a particular embodiment, an antigen binding protein is administered by subconjunctival injection. In a particular embodiment, an antigen binding protein of the invention is administered topically. In another embodiment, an antigen binding protein of the invention is administered via a sustained release drug delivery system. In a particular embodiment, an antigen binding protein of the invention is administered via intravenous injection. In a particular embodiment, an antigen binding protein of the invention is administered via subcutaneous injection.

In a particular embodiment of the invention, the antigen binding protein is DMS4000 or an antigen binding protein consisting of a heavy chain sequence of SEQ ID NO:69, 70, 71 or 72 and a light chain sequence of SEQ ID NO:12, which is to be administered by intravitreal injection every 4-8 weeks.

Therapeutic agents of the invention may be prepared as pharmaceutical compositions containing an effective amount of the antigen-binding protein of the invention as an active ingredient in a pharmaceutically acceptable carrier. In the prophylactic agent of the invention, an aqueous suspension or solution containing the antigen-binding protein, may be buffered at physiological pH, in a form ready for injection. The compositions for parenteral administration will commonly comprise a solution of the antigen-binding protein of the invention or a cocktail thereof dissolved in a pharmaceutically acceptable carrier, for example an aqueous carrier. A variety of aqueous carriers may be employed, e.g., 0.9% saline, 0.3% glycine, and the like. These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antigen-binding protein of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 200 mg, e.g. about 50 ng to about 30 mg or more, or about 5 mg to about 25 mg, of an antigen-binding protein of the invention. Similarly, a pharmaceutical composition of the invention for intravenous infusion could be made up to contain about 250 ml of sterile Ringer's solution, and about 1 to about 30 or about 5 mg to about 25 mg of an antigen-binding protein of the invention per ml of Ringer's solution. Actual methods for preparing parenterally administrable compositions are well known or will be apparent to those skilled in the art and are described in more detail in, for example, Remington's Pharmaceutical Science, 15th ed., Mack Publishing Company, Easton, Pa. For the preparation of intravenously administrable antigen-binding protein formulations of the invention see Lasmar U and Parkins D “The formulation of Biopharmaceutical products”, Pharma. Sci. Tech. today, page 129-137, Vol. 3 (3 Apr. 2000); Wang, W “Instability, stabilisation and formulation of liquid protein pharmaceuticals”, Int. J. Pharm 185 (1999) 129-188; Stability of Protein Pharmaceuticals Part A and B ed Ahern T. J., Manning M. C., New York, N.Y.: Plenum Press (1992); Akers, M. J. “Excipient-Drug interactions in Parenteral Formulations”, J. Pharm Sci 91 (2002) 2283-2300; Imamura, K et al., “Effects of types of sugar on stabilization of Protein in the dried state”, J Pharm Sci 92 (2003) 266-274; Izutsu, Kkojima, S. “Excipient crystallinity and its protein-structure-stabilizing effect during freeze-drying”, J. Pharm. Pharmacol, 54 (2002) 1033-1039; Johnson, R, “Mannitol-sucrose mixtures-versatile formulations for protein lyophilization”, J. Pharm. Sci, 91 (2002) 914-922; Ha, E Wang W, Wang Y. j. “Peroxide formation in polysorbate 80 and protein stability”, J. Pharm Sci, 91, 2252-2264, (2002) the entire contents of which are incorporated herein by reference and to which the reader is specifically referred.

In one embodiment the therapeutic agent of the invention, when in a pharmaceutical preparation, is present in unit dose forms. The appropriate therapeutically effective dose will be determined readily by those of skill in the art. Suitable doses may be calculated for patients according to their weight, for example suitable doses may be in the range of 0.00001 to 20 mg/kg, for example 0.0001 to 20 mg/kg, for example 0.1 to 20 mg/kg, for example 1 to 20 mg/kg or for example 1 to 15 mg/kg, for example 10 to 15 mg/kg. To effectively treat conditions of use in the present invention in a human, suitable doses may be within the range of 0.0001 to 1000 mg, for example 0.001 to 1000 mg, for example 0.01 to 500 mg, for example 500 mg, for example 0.1 to 100 mg, or 0.1 to 80 mg, or 0.1 to 60 mg, or 0.1 to 40 mg, or for example 1 to 100 mg, or 1 to 50 mg, of an antigen-binding protein of this invention, which may be administered parenterally, for example subcutaneously, intravenously or intramuscularly; or topically. Such dose may, if necessary, be repeated at appropriate time intervals selected as appropriate by a physician.

Where the therapeutic agent is to be administered directly into the eye, e.g. by intravitreal injection, it is preferable that the dosage should be such that the total amount of protein administered to each human eye does not exceed 2 mg. In an embodiment the total amount of protein administered to a single human eye is approximately 2 mg. In an embodiment the total amount of protein administered to a single human eye is approximately 1.8 mg. In an embodiment the total amount of protein administered to a single human eye is approximately 1.6 mg. In an embodiment the total amount of protein administered to a single human eye is approximately 1.4 mg. In an embodiment the total amount of protein administered to a single human eye is approximately 1.2 mg. In an embodiment the total amount of protein administered to a single human eye is approximately 1.0 mg. In an embodiment, the total amount of protein administered to a single human eye is less than 2.0 mg, less than 1.8 mg, less than 1.6 mg, less than 1.4 mg, less than 1.2 mg, or less than 1.0 mg.

The antigen-binding proteins described herein can be lyophilized for storage and reconstituted in a suitable carrier prior to use. This technique has been shown to be effective with conventional immunoglobulins and art-known lyophilization and reconstitution techniques can be employed.

There are several methods known in the art which can be used to find epitope-binding domains of use in the present invention.

The term “library” refers to a mixture of heterogeneous polypeptides or nucleic acids. The library is composed of members, each of which has a single polypeptide or nucleic acid sequence. To this extent, “library” is synonymous with “repertoire.” Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. In one example, each individual organism or cell contains only one or a limited number of library members. Advantageously, the nucleic acids are incorporated into expression vectors, in order to allow expression of the polypeptides encoded by the nucleic acids. In a one aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of diverse polypeptides.

A “universal framework” is a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, J. Mol. Biol. (1987) 196: 910-917. There may be a single framework, or a set of such frameworks, which has been found to permit the derivation of virtually any binding specificity though variation in the hypervariable regions alone.

Amino acid and nucleotide sequence alignments and homology, similarity or identity, as defined herein are in one embodiment prepared and determined using the algorithm BLAST 2 Sequences, using default parameters (Tatusova, T. A. et al., FEMS Microbiol Lett, (1999) 174: 187-188).

When a display system (e.g., a display system that links coding function of a nucleic acid and functional characteristics of the peptide or polypeptide encoded by the nucleic acid) is used in the methods described herein, e.g. in the selection of a dAb or other epitope binding domain, it is frequently advantageous to amplify or increase the copy number of the nucleic acids that encode the selected peptides or polypeptides. This provides an efficient way of obtaining sufficient quantities of nucleic acids and/or peptides or polypeptides for additional rounds of selection, using the methods described herein or other suitable methods, or for preparing additional repertoires (e.g., affinity maturation repertoires). Thus, in some embodiments, the methods of selecting epitope binding domains comprises using a display system (e.g., that links coding function of a nucleic acid and functional characteristics of the peptide or polypeptide encoded by the nucleic acid, such as phage display) and further comprises amplifying or increasing the copy number of a nucleic acid that encodes a selected peptide or polypeptide. Nucleic acids can be amplified using any suitable methods, such as by phage amplification, cell growth or polymerase chain reaction.

In one example, the methods employ a display system that links the coding function of a nucleic acid and physical, chemical and/or functional characteristics of the polypeptide encoded by the nucleic acid. Such a display system can comprise a plurality of replicable genetic packages, such as bacteriophage or cells (bacteria). The display system may comprise a library, such as a bacteriophage display library. Bacteriophage display is an example of a display system.

A number of suitable bacteriophage display systems (e.g., monovalent display and multivalent display systems) have been described. (See, e.g., Griffiths et al., U.S. Pat. No. 6,555,313 B1 (incorporated herein by reference); Johnson et al., U.S. Pat. No. 5,733,743 (incorporated herein by reference); McCafferty et al., U.S. Pat. No. 5,969,108 (incorporated herein by reference); Mulligan-Kehoe, U.S. Pat. No. 5,702,892 (Incorporated herein by reference); Winter, G. et al., Annu. Rev. Immunol. (1994) 12: 433-455; Soumillion, P. et al., Appl. Biochem. Biotechnol. (1994) 47(2-3): 175-189; Castagnoli, L. et al., Comb. Chem. High Throughput Screen (2001) 4(2): 121-133) The peptides or polypeptides displayed in a bacteriophage display system can be displayed on any suitable bacteriophage, such as a filamentous phage (e.g., fd, M13, F1), a lytic phage (e.g., T4, T7, lambda), or an RNA phage (e.g., MS2), for example.

Generally, a library of phage that displays a repertoire of peptides or phagepolypeptides, as fusion proteins with a suitable phage coat protein (e.g., fd pIII protein), is produced or provided. The fusion protein can display the peptides or polypeptides at the tip of the phage coat protein, or if desired at an internal position. For example, the displayed peptide or polypeptide can be present at a position that is amino-terminal to domain 1 of pIII. (Domain 1 of pIII is also referred to as N1.) The displayed polypeptide can be directly fused to pIII (e.g., the N-terminus of domain 1 of pIII) or fused to pIII using a linker. If desired, the fusion can further comprise a tag (e.g., myc epitope, His tag). Libraries that comprise a repertoire of peptides or polypeptides that are displayed as fusion proteins with a phage coat protein can be produced using any suitable methods, such as by introducing a library of phage vectors or phagemid vectors encoding the displayed peptides or polypeptides into suitable host bacteria, and culturing the resulting bacteria to produce phage (e.g., using a suitable helper phage or complementing plasmid if desired). The library of phage can be recovered from the culture using any suitable method, such as precipitation and centrifugation.

The display system can comprise a repertoire of peptides or polypeptides that contains any desired amount of diversity. For example, the repertoire can contain peptides or polypeptides that have amino acid sequences that correspond to naturally occurring polypeptides expressed by an organism, group of organisms, desired tissue or desired cell type, or can contain peptides or polypeptides that have random or randomized amino acid sequences. If desired, the polypeptides can share a common core or scaffold. For example, all polypeptides in the repertoire or library can be based on a scaffold selected from protein A, protein L, protein G, a fibronectin domain, an anticalin, CTLA4, a desired enzyme (e.g., a polymerase, a cellulase), or a polypeptide from the immunoglobulin superfamily, such as an antibody or antibody fragment (e.g., an antibody variable domain). The polypeptides in such a repertoire or library can comprise defined regions of random or randomized amino acid sequence and regions of common amino acid sequence. In certain embodiments, all or substantially all polypeptides in a repertoire are of a desired type, such as a desired enzyme (e.g., a polymerase) or a desired antigen-binding fragment of an antibody (e.g., human V_{H} or human V_{L}). In some embodiments, the polypeptide display system comprises a repertoire of polypeptides wherein each polypeptide comprises an antibody variable domain. For example, each polypeptide in the repertoire can contain a V_{H}, a V_{L} or an Fv (e.g., a single chain Fv).

Amino acid sequence diversity can be introduced into any desired region of a peptide or polypeptide or scaffold using any suitable method. For example, amino acid sequence diversity can be introduced into a target region, such as a complementarity determining region of an antibody variable domain or a hydrophobic domain, by preparing a library of nucleic acids that encode the diversified polypeptides using any suitable mutagenesis methods (e.g., low fidelity PCR, oligonucleotide-mediated or site directed mutagenesis, diversification using NNK codons) or any other suitable method. If desired, a region of a polypeptide to be diversified can be randomized. The size of the polypeptides that make up the repertoire is largely a matter of choice and uniform polypeptide size is not required. The polypeptides in the repertoire may have at least tertiary structure (i.e. form at least one domain).

### Selection/Isolation/Recovery

An epitope binding domain or population of domains can be selected, isolated and/or recovered from a repertoire or library (e.g., in a display system) using any suitable method. For example, a domain is selected or isolated based on a selectable characteristic (e.g., physical characteristic, chemical characteristic, functional characteristic). Suitable selectable functional characteristics include biological activities of the peptides or polypeptides in the repertoire, for example, binding to a generic ligand (e.g., a superantigen), binding to a target ligand (e.g., an antigen, an epitope, a substrate), binding to an antibody (e.g., through an epitope expressed on a peptide or polypeptide), and catalytic activity. (See, e.g., Tomlinson et al., WO 99/20749; WO 01/57065; WO 99/58655.)

In some embodiments, the protease resistant peptide or polypeptide is selected and/or isolated from a library or repertoire of peptides or polypeptides in which substantially all domains share a common selectable feature. For example, the domain can be selected from a library or repertoire in which substantially all domains bind a common generic ligand, bind a common target ligand, bind (or are bound by) a common antibody, or possess a common catalytic activity. This type of selection is particularly useful for preparing a repertoire of domains that are based on a parental peptide or polypeptide that has a desired biological activity, for example, when performing affinity maturation of an immunoglobulin single variable domain. Selection based on binding to a common generic ligand can yield a collection or population of domains that contain all or substantially all of the domains that were components of the original library or repertoire. For example, domains that bind a target ligand or a generic ligand, such as protein A, protein L or an antibody, can be selected, isolated and/or recovered by panning or using a suitable affinity matrix. Panning can be accomplished by adding a solution of ligand (e.g., generic ligand, target ligand) to a suitable vessel (e.g., tube, petri dish) and allowing the ligand to become deposited or coated onto the walls of the vessel. Excess ligand can be washed away and domains can be added to the vessel and the vessel maintained under conditions suitable for peptides or polypeptides to bind the immobilized ligand. Unbound domains can be washed away and bound domains can be recovered using any suitable method, such as scraping or lowering the pH, for example. Suitable ligand affinity matrices generally contain a solid support or bead (e.g., agarose) to which a ligand is covalently or noncovalently attached. The affinity matrix can be combined with peptides or polypeptides (e.g., a repertoire that has been incubated with protease) using a batch process, a column process or any other suitable process under conditions suitable for binding of domains to the ligand on the matrix. Domains that do not bind the affinity matrix can be washed away and bound domains can be eluted and recovered using any suitable method, such as elution with a lower pH buffer, with a mild denaturing agent (e.g., urea), or with a peptide or domain that competes for binding to the ligand. In one example, a biotinylated target ligand is combined with a repertoire under conditions suitable for domains in the repertoire to bind the target ligand. Bound domains are recovered using immobilized avidin or streptavidin (e.g., on a bead).

In some embodiments, the generic or target ligand is an antibody or antigen binding fragment thereof. Antibodies or antigen binding fragments that bind structural features of peptides or polypeptides that are substantially conserved in the peptides or polypeptides of a library or repertoire are particularly useful as generic ligands. Antibodies and antigen binding fragments suitable for use as ligands for isolating, selecting and/or recovering protease resistant peptides or polypeptides can be monoclonal or polyclonal and can be prepared using any suitable method.

### Libraries/Repertoires

Libraries that encode and/or contain epitope binding domains can be prepared or obtained using any suitable method. A library can be designed to encode domains based on a domain or scaffold of interest (e.g., a domain selected from a library) or can be selected from another library using the methods described herein. For example, a library enriched in domains can be prepared using a suitable polypeptide display system.

Libraries that encode a repertoire of a desired type of domain can readily be produced using any suitable method. For example, a nucleic acid sequence that encodes a desired type of polypeptide (e.g., an immunoglobulin variable domain) can be obtained and a collection of nucleic acids that each contain one or more mutations can be prepared, for example by amplifying the nucleic acid using an error-prone polymerase chain reaction (PCR) system, by chemical mutagenesis (Deng et al., J. Biol. Chem., 269:9533 (1994)) or using bacterial mutator strains (Low et al., J. Mol. Biol., 260:359 (1996)).

In other embodiments, particular regions of the nucleic acid can be targeted for diversification. Methods for mutating selected positions are also well known in the art and include, for example, the use of mismatched oligonucleotides or degenerate oligonucleotides, with or without the use of PCR. For example, synthetic antibody libraries have been created by targeting mutations to the antigen binding loops. Random or semi-random antibody H3 and L3 regions have been appended to germline immunobulin V gene segments to produce large libraries with unmutated framework regions (Hoogenboom and Winter (1992) supra; Nissim et al. (1994) supra; Griffiths et al. (1994) supra; DeKruif et al. (1995) supra). Such diversification has been extended to include some or all of the other antigen binding loops (Crameri et al. Nature Med. (1996) 2: 100; Riechmann et al. Bio/Technology (1995) 13: 475; Morphosys, WO 97/08320, supra). In other embodiments, particular regions of the nucleic acid can be targeted for diversification by, for example, a two-step PCR strategy employing the product of the first PCR as a “mega-primer.” (See, e.g., Landt, O. et al., Gene (1990) 96: 125-128) Targeted diversification can also be accomplished, for example, by SOE PCR. (See, e.g., Horton, R. M. et al., Gene (1989) 77: 61-68)

Sequence diversity at selected positions can be achieved by altering the coding sequence which specifies the sequence of the polypeptide such that a number of possible amino acids (e.g., all 20 or a subset thereof) can be incorporated at that position. Using the IUPAC nomenclature, the most versatile codon is NNK, which encodes all amino acids as well as the TAG stop codon. The NNK codon may be used in order to introduce the required diversity. Other codons which achieve the same ends are also of use, including the NNN codon, which leads to the production of the additional stop codons TGA and TAA. Such a targeted approach can allow the full sequence space in a target area to be explored.

Some libraries comprise domains that are members of the immunoglobulin superfamily (e.g., antibodies or portions thereof). For example the libraries can comprise domains that have a known main-chain conformation. (See, e.g., Tomlinson et al., WO 99/20749.)

Libraries can be prepared in a suitable plasmid or vector. As used herein, vector refers to a discrete element that is used to introduce heterologous DNA into cells for the expression and/or replication thereof. Any suitable vector can be used, including plasmids (e.g., bacterial plasmids), viral or bacteriophage vectors, artificial chromosomes and episomal vectors. Such vectors may be used for simple cloning and mutagenesis, or an expression vector can be used to drive expression of the library. Vectors and plasmids usually contain one or more cloning sites (e.g., a polylinker), an origin of replication and at least one selectable marker gene. Expression vectors can further contain elements to drive transcription and translation of a polypeptide, such as an enhancer element, promoter, transcription termination signal, signal sequences, and the like. These elements can be arranged in such a way as to be operably linked to a cloned insert encoding a polypeptide, such that the polypeptide is expressed and produced when such an expression vector is maintained under conditions suitable for expression (e.g., in a suitable host cell).

Cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV40, adenovirus) are useful for cloning vectors in mammalian cells. Generally, the origin of replication is not needed for mammalian expression vectors, unless these are used in mammalian cells able to replicate high levels of DNA, such as COS cells.

Cloning or expression vectors can contain a selection gene also referred to as selectable marker. Such marker genes encode a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal or leader sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., the β-lactamase and lactose promoter systems, alkaline phosphatase, the tryptophan (trp) promoter system, lac, tac, T3, T7 promoters for E. coli) and eucaryotic (e.g., simian virus 40 early or late promoter, Rous sarcoma virus long terminal repeat promoter, cytomegalovirus promoter, adenovirus late promoter, EG-1a promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in procaryotic (e.g., β-lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated.

Suitable expression vectors for expression in prokaryotic (e.g., bacterial cells such as E. coli) or mammalian cells include, for example, a pET vector (e.g., pET-12a, pET-36, pET-37, pET-39, pET-40, Novagen and others), a phage vector (e.g., pCANTAB 5 E, Pharmacia), pRIT2T (Protein A fusion vector, Pharmacia), pCDM8, pcDNA1.1/amp, pcDNA3.1, pRc/RSV, pEF-1 (Invitrogen, Carlsbad, Calif.), pCMV-SCRIPT, pFB, pSG5, pXT1 (Stratagene, La Jolla, Calif.), pCDEF3 (Goldman, L. A., et al., Biotechniques, 21:1013-1015 (1996)), pSVSPORT (GibcoBRL, Rockville, Md.), pEF-Bos (Mizushima, S., et al., Nucleic Acids Res. (1990) 18: 5322) and the like. Expression vectors which are suitable for use in various expression hosts, such as prokaryotic cells (E. coli), insect cells (Drosophila Schnieder S2 cells, Sf9), yeast (P. methanolica, P. pastoris, S. cerevisiae) and mammalian cells (eg, COS cells) are available.

Some examples of vectors are expression vectors that enable the expression of a nucleotide sequence corresponding to a polypeptide library member. Thus, selection with generic and/or target ligands can be performed by separate propagation and expression of a single clone expressing the polypeptide library member. As described above, a particular selection display system is bacteriophage display. Thus, phage or phagemid vectors may be used, for example vectors may be phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector can contain a β-lactamase gene to confer selectivity on the phagemid and a lac promoter upstream of an expression cassette that can contain a suitable leader sequence, a multiple cloning site, one or more peptide tags, one or more TAG stop codons and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Antibody variable domains may comprise a target ligand binding site and/or a generic ligand binding site. In certain embodiments, the generic ligand binding site is a binding site for a superantigen, such as protein A, protein L or protein G. The variable domains can be based on any desired variable domain, for example a human VH (e.g., V_{H}1a, V_{H}1b, V_{H}2, V_{H}3, V_{H}4, V_{H}5, V_{H}6), a human Vλ (e.g., VλI, VλII, VλIII, VλIV, VλV, VλVI or Vκ1) or a human Vκ (e.g., Vκ2, Vκ3, Vκ4, Vκ5, Vκ6, Vκ7, Vκ8, Vκ9 or Vκ10).

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths Nature Biotechnol (1998) 16(7): 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

### Characterisation of the Epitope Binding Domains.

The binding of a domain to its specific antigen or epitope can be tested by methods which will be familiar to those skilled in the art and include ELISA. In one example, binding is tested using monoclonal phage ELISA.

Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. Ann. Rev. Immunology (1994) 12: 433-55 and references cited therein.

The diversity of the selected phage monoclonal antibodies may also be assessed by gel electrophoresis of PCR products and probing (Marks et al. 1991, supra; Nissim et al. 1994 supra), (Tomlinson et al., 1992) J. Mol. Biol. 227, 776) or by sequencing of the vector DNA or restriction digets analysis with a frequent cutter such as BSTNI.

Structure of dAbs

In the case that the dAbs are selected from V-gene repertoires selected for instance using phage display technology as herein described, then these variable domains comprise a universal framework region, such that is they may be recognised by a specific generic ligand as herein defined. The use of universal frameworks, generic ligands and the like is described in WO99/20749.

Where V-gene repertoires are used variation in polypeptide sequence may be located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

Scaffolds for Use in Constructing dAbs
i. Selection of the Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk J. Mol. Biol. (1987) 196: 901; Chothia et al. Nature (1989) 342: 877). Analysis of loop lengths and key residues has therefore enabled prediction of the main-chain conformations of H1, H2, L1, L2 and L3 found in the majority of human antibodies (Chothia et al. J. Mol. Biol. (1992) 227: 799; Tomlinson et al. EMBO J. (1995) 14: 4628; Williams et al. J. Mol. Biol. (1996) 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. J. Mol. Biol. (1996) 263: 800; Shirai et al. FEBS Letters (1996) 399: 1).

The dAbs are advantageously assembled from libraries of domains, such as libraries of V_{H} domains and/or libraries of V_{L} domains. In one aspect, libraries of domains are designed in which certain loop lengths and key residues have been chosen to ensure that the main-chain conformation of the members is known. Advantageously, these are real conformations of immunoglobulin superfamily molecules found in nature, to minimise the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on ligand sequences and to chose residues for diversification which do not affect the canonical structure. It is known that, in the human V_{K} domain, the L1 loop can adopt one of four canonical structures, the L2 loop has a single canonical structure and that 90% of human V_{K} domains adopt one of four or five canonical structures for the L3 loop (Tomlinson et al. (1995) supra); thus, in the V_{K} domain alone, different canonical structures can combine to create a range of different main-chain conformations. Given that the Vλ domain encodes a different range of canonical structures for the L1, L2 and L3 loops and that V_{K} and Vλ domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a one particular aspect, the dAbs possess a single known main-chain conformation.

The single main-chain conformation that is chosen may be commonplace among molecules of the immunoglobulin superfamily type in question. A conformation is commonplace when a significant number of naturally occurring molecules are observed to adopt it. Accordingly, in one aspect, the natural occurrence of the different main-chain conformations for each binding loop of an immunoglobulin domain are considered separately and then a naturally occurring variable domain is chosen which possesses the desired combination of main-chain conformations for the different loops. If none is available, the nearest equivalent may be chosen. The desired combination of main-chain conformations for the different loops may be created by selecting germline gene segments which encode the desired main-chain conformations. In one example, the selected germline gene segments are frequently expressed in nature, and in particular they may be the most frequently expressed of all natural germline gene segments.

In designing libraries the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e. between 35% and 100% and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1—CS1 (79% of the expressed repertoire), H2—CS 3 (46%), L1—CS 2 of V_{K} (39%), L2—CS1 (100%), L3—CS1 of V_{K} (36%) (calculation assumes a κ:λ ratio of 70:30, Hood et al., Cold Spring Harbor Symp. Quant. Biol. (1967) 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}1. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five, or for all six of the antigen binding loops can be determined. Here, the chosen conformation may be commonplace in naturally occurring antibodies and may be observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

### Diversification of the Canonical Sequence

Having selected several known main-chain conformations or a single known main-chain conformation, dAbs can be constructed by varying the binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or they may be selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al., J. Mol. Biol. (1992) 226: 889), chemical mutagenesis (Deng et al., J. Biol. Chem. (1994) 269: 9533) or bacterial mutator strains (Low et al., J. Mol. Biol. (1996) 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al., Proc. Natl. Acad. Sci. USA (1992) 89: 4457). Random or semi-random H3 and L3 regions have been appended to germline V gene segments to produce large libraries with unmutated framework regions (Hoogenboom & Winter J. Mol. Biol. (1992) 227: 381; Barbas et al., Proc. Natl. Acad. Sci. USA (1992) 89: 4457; Nissim et al., EMBO J. (1994) 13: 692; Griffiths et al. EMBO J. (1994) 13: 3245; De Kruif et al, J. Mol. Biol. (1995) 248: 97). Such diversification has been extended to include some or all of the other antigen binding loops (Crameri et al. Nature Med. (1996) 2: 100; Riechmann et al. Bio/Technology (1995) 13: 475; Morphosys, WO97/08320, supra).

Since loop randomisation has the potential to create approximately more than 10¹⁵ structures for H3 alone and a similarly large number of variants for the other five loops, it is not feasible using current transformation technology or even by using cell free systems to produce a library representing all possible combinations. Even for some of the largest libraries constructed in excess of 6×10¹² different antibodies, using technologies such as ribosomal display, only a fraction of the potential diversity would be represented in a library of this design (He and Taussig, Nucleic Acid Research 1997 25(24): 5132).

In a one embodiment, only those residues which are directly involved in creating or modifying the desired function of the molecule are diversified. For many molecules, the function will be to bind a target and therefore diversity should be concentrated in the target binding site, while avoiding changing residues which are crucial to the overall packing of the molecule or to maintaining the chosen main-chain conformation.

In one aspect, libraries of dAbs are used in which only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library. This represents a significant improvement in terms of the functional diversity required to create a range of antigen binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al., J. Mol. Biol. (1996) 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire is created where some, but not all, of the residues in the antigen binding site are diversified. As used herein in this context, the term “naive” or “dummy” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

It will be understood that the sequences described herein include sequences which are substantially identical, for example sequences which are at least 90% identical, for example which are at least 91%, or at least 92%, or at least 93%, or at least 94% or at least 95%, or at least 96%, or at least 97% or at least 98%, or at least 99% identical to the sequences described herein.

For nucleic acids, the term “substantial identity” indicates that two nucleic acids, or designated sequences thereof, when optimally aligned and compared, are identical, with appropriate nucleotide insertions or deletions, in at least about 80% of the nucleotides, usually at least about 90% to 95%, or at least about 98% to 99.5% of the nucleotides. Alternatively, substantial identity exists when the segments will hybridize under selective hybridization conditions, to the complement of the strand.

For nucleotide and amino acid sequences, the term “identical” indicates the degree of identity between two nucleic acid or amino acid sequences when optimally aligned and compared with appropriate insertions or deletions. Alternatively, substantial identity exists when the DNA segments will hybridize under selective hybridization conditions, to the complement of the strand.

The percent identity between two sequences is a function of the number of identical positions shared by the sequences (i.e., % identity=number of identical positions/total number of positions, times 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

By way of example, a polypeptide sequence of the present invention may be identical to the reference sequence encoded by SEQ ID NO: 14, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by SEQ ID NO: 14 by the numerical percent of the respective percent identity (divided by 100) and then subtracting that product from said total number of amino acids in the polypeptide sequence encoded by SEQ ID NO: 14, or:

na≦xa−(xa·y),

wherein na is the number of amino acid alterations, xa is the total number of amino acids in the polypeptide sequence encoded by SEQ ID NO: 14, and y is, for instance 0.70 for 70%, 0.80 for 80%, 0.85 for 85% etc., and wherein any non-integer product of xa and y is rounded down to the nearest integer prior to subtracting it from xa.

### EXAMPLES

### Example 1

1.1 Generation of a Dual Targeting Anti-TNFα/Anti-VEGF mAbdAb (DMS4000)

An anti-TNFα/anti-VEGF mAbdAb (designated DMS4000) was produced by fusion of a dAb to the C-terminus of the mAb (adalimumab) heavy chain. For construction of the heavy chain expression cassette, vector DNA encoding the heavy chain of an alternative mAbdAb was taken as a starting point. The dAb portion was excised using the restriction enzymes SalI and HindIII. DOM15-26-593, an anti-VEGF dAb, was amplified by PCR (using primers coding SalI and HindIII ends) and ligated into the vector backbone from which the dAb had been excised using the same restriction sites, resulting in a linker of ‘STG’ (serine, threonine, glycine) between the mAb and the dAb.

Sequence verified clones (SEQ ID NO:11 and 13 for light and heavy chains respectively) were selected and large scale DNA preparations were made and the anti-TNFα/anti-VEGF mAbdAb was expressed in mammalian HEK293-6E cells (National Research Council Canada) using transient transfection techniques by co-transfection of light and heavy chains (SEQ ID NO:12 and 14).

The sequence of the anti-TNFα/anti-VEGF mAbdAb heavy chain was further modified to have a codon optimised sequence for the anti VEGF dAb, and incorporate L235A and G237A mutations (Kabat numbering) to disable the FC effector function (DMS4000 mAbdAb heavy chain Fc disabled SEQ ID NO 46 and 47).

1.2 Purification and SEC Analysis of the Dual Targeting Anti-TNFα/Anti-VEGF mAbdAb (DMS4000)

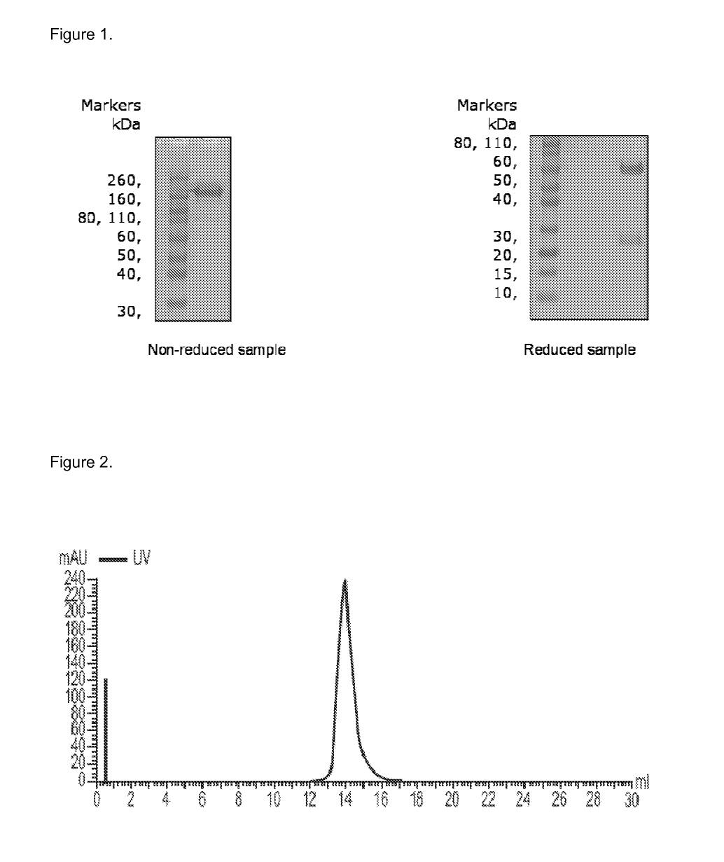

The anti-TNFα/anti-VEGF mAbdAb (designated DMS4000) was purified from clarified expression supernatant using Protein-A affinity chromatography according to established protocols. Concentrations of purified samples were determined by spectrophotometry from measurements of light absorbance at 280 nm. SDS-PAGE analysis (FIG. 1) of the purified sample shows non-reduced sample running at ˜170 kDa whilst reduced sample shows two bands running at ˜25 and ˜60 kDa corresponding to light chain and dAb-fused heavy chain respectively.

For size exclusion chromatography (SEC) analysis the anti-TNFα/anti-VEGF mAbdAb was applied onto a Superdex-200 10/30 HR column (attached to an Akta Express FPLC system) pre-equilibrated and running in PBS at 0.5 ml/min. The SEC profile shows a single species running as a symmetrical peak (FIG. 2).

1.3 Binding Affinities of the Dual Targeting Anti-TNFα/Anti-VEGF mAbdAb DMS4000

### VEGF Receptor Binding Assay.

This assay measures the binding of VEGF₁₆₅ to VEGF R2 (VEGF receptor) and the ability of test molecules to block this interaction. ELISA plates were coated overnight with VEGF receptor (R&D Systems, Cat No: 357-KD-050) (0.5 μg/ml final concentration in 0.2M sodium carbonate bicarbonate pH9.4), washed and blocked with 2% BSA in PBS. VEGF (R&D Systems, Cat No: 293-VE-050) and the test molecules (diluted in 0.1% BSA in 0.05% Tween 20™ PBS) were pre-incubated for one hour prior to addition to the plate (3 ng/ml VEGF final concentration). Binding of VEGF to VEGF receptor was detected using biotinylated anti-VEGF antibody (0.5 μg/ml final concentration) (R&D Systems, Cat No: BAF293) and a peroxidase conjugated anti-biotin secondary antibody (1:5000 dilution) (Stratech, Cat No: 200-032-096) and visualised at OD450 using a colorimetric substrate (Sure Blue TMB peroxidase substrate, KPL) after stopping the reaction with an equal volume of 1M HCl.

### MRC-5/TNFα Assay

The ability of test molecules to prevent human TNFα binding to human TNFR1 and neutralise IL-8 secretion was determined using human lung fibroblast MRC-5 cells. A dilution series of test samples was incubated with TNFα (500 μg/ml) (Peprotech) for 1 hour. This was then diluted 1 in 2 with a suspension of MRC-5 cells (ATCC, Cat. #CCL-171) (5×10³ cells/well). After an overnight incubation, samples were diluted 1 in 10, and IL-8 release was determined using an IL-8 ABI 8200 cellular detection assay (FMAT) where the IL-8 concentration was determined using anti-IL-8 (R&D systems, Cat#208-IL) coated polystyrene beads, biotinylated anti-IL-8 (R&D systems, Cat#BAF208) and streptavidin Alexafluor 647 (Molecular Probes, Cat#S32357). The assay readout was localised fluorescence emission at 647 nm and unknown IL-8 concentrations were interpolated using an IL-8 standard curve included in the assay.

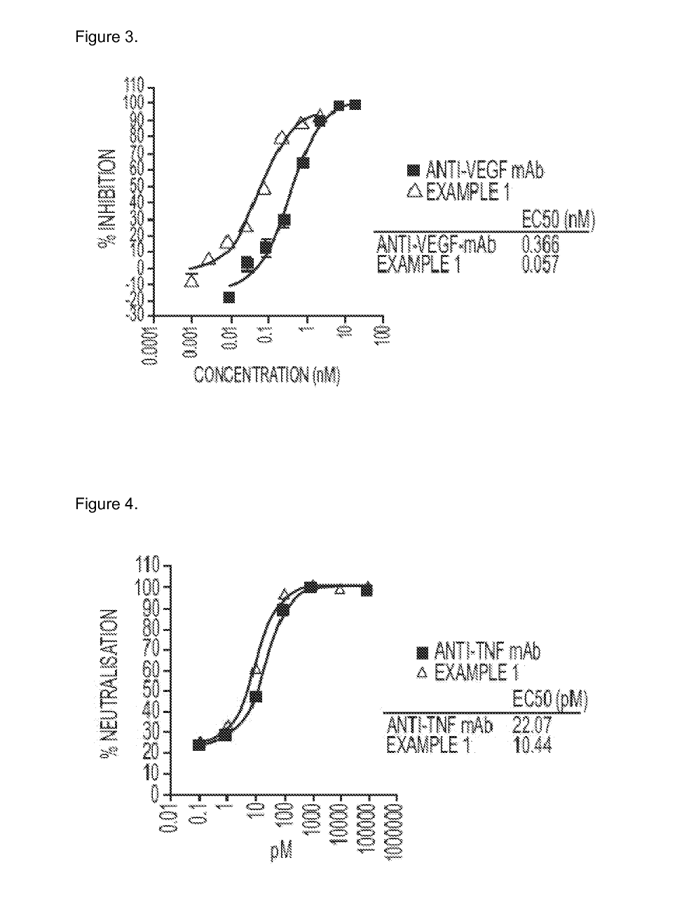

Binding affinities to VEGF and TNFα were determined as described as set out above. Assay data were analysed using GraphPad Prism. Potency values were determined using a sigmoidal dose response curve and the data fitted using the best fit model. Anti-VEGF potency (FIG. 3) of this mAbdAb was calculated to be 57 pM whilst the control, an anti-VEGF mAb, gave an EC50 value of 366 pM. In the anti-TNFα bioassay (FIG. 4) the potency was 10 pM whilst an anti-TNFα control mAb produced an EC50 of 22 pM. In conclusion, assay data shows that this dual targeting mAbdAb is potent against both antigens (TNFα and VEGF).

1.4 Rat PK of the Dual Targeting Anti-TNFα/Anti-VEGF mAbdAb (DMS4000)

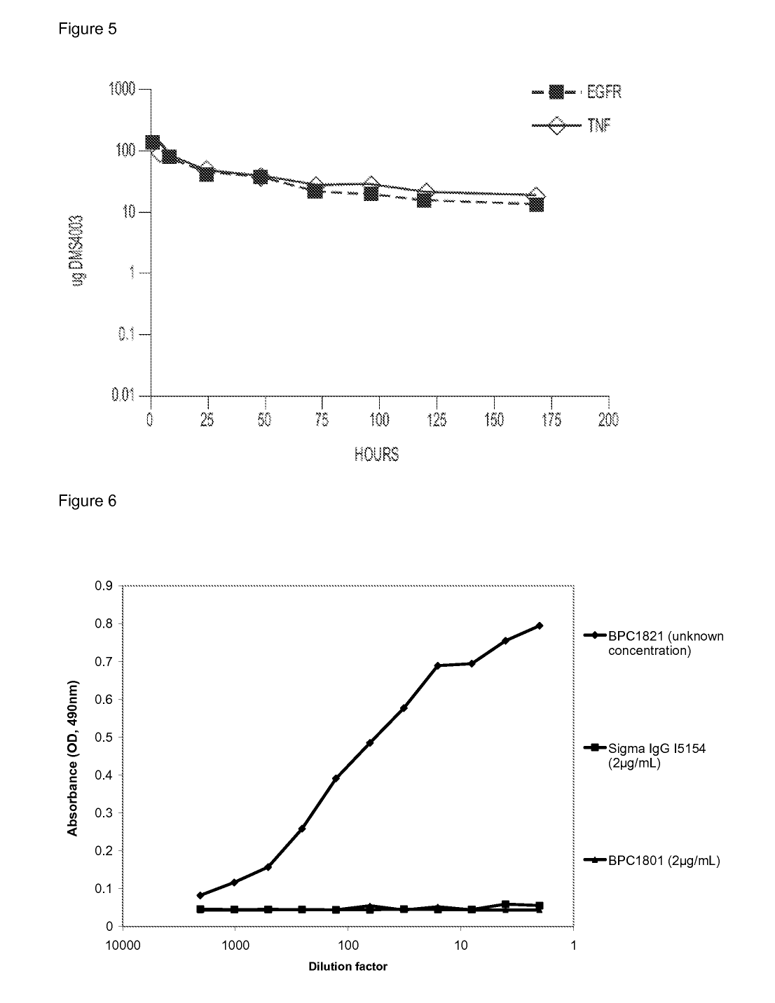

This molecule was tested for its in vivo pharmacokinetic properties in the rat. The anti-TNFα/anti-VEGF mAbdAb was administered i.v. to three rats, and serum samples collected over a period of 10 days (240 hours). The concentration of drug remaining at various time points post-dose was assessed by ELISA against both TNFα & VEGF. The results are shown in FIG. 5.

The PK parameters confirmed that this molecule had in vivo pharmacokinetic properties that compared with those of an anti-TNFα mAb. The shorter observed t_{1/2}β for the VEGF component is not considered to be significant and may be an assay artefact. Further details are shown in Table 3.

TABLE 3

| | Half Life | Cmax | AUC (0-inf) | Clearance | % AUC |
|---|---|---|---|---|---|
| Antigen | (hr) | (μg/mL) | (hr* μg/mL) | (mL/hr/kg) | Extrapolated |
| TNFα | 180.1 | 89.9 | 7286.3 | 0.7 | 35.8 |
| VEGF | 94.2 | 102.8 | 4747.1 | 1.1 | 14.3 |

1.5 Generation of an Alternative Anti-TNFα/Anti-VEGF mAbdAb (DMS4031)

An alternative anti-TNFα/anti-VEGF mAbdAb (designated DMS4031) was constructed in a similar way to that described above in Example 1.1, using the same anti-TNFα mAb (adalimumab) linked to a VEGF dAb on the C-terminus of the heavy chain using an STG linker. The anti-VEGF dAb used in this case was DOM15-10-11. This molecule was expressed in mammalian HEK293-6E cells (National Research Council Canada) using transient transfection techniques by co-transfection of light and heavy chains (SEQ ID NO:12 and 16). This molecule expressed to give a mAbdAb of similar expression levels to that described in Example 1.2, however when tested for potency in the same VEGF assay as described in Example 1.3 it was found to have undetectable levels of inhibition of VEGF binding to VEGF receptor in this assay.

### Example 2

Biacore Analysis of Dual Targeting Anti-TNFα/Anti-VEGF mAbdAbs

The test mAbdAb was subjected to BIAcore analysis to determine kinetic association and dissociation constants for binding to their corresponding antigens. Analysis was performed on BIAcore™ 3000 instrument. The temperature of the instrument was set to 25° C. HBS-EP buffer was used as running buffer. Experimental data were collected at the highest possible rate for the instrument. One flow cell on a research grade CM5 chip was coated with protein A using standard amine coupling chemistry according to manufacturer's instructions, and a second flow cell was treated equally but buffer was used instead of protein A to generate a reference surface. The flow cell coated with protein A was then used to capture mAbdAbs. Antigen was injected as a series 2× serial dilutions as detailed in table 2. Several dilutions were run in duplicate. Injections of buffer alone instead of ligand were used for background subtraction. Samples were injected in random order using the kinetics Wizard inherent to the instrument software. The surface was regenerated at the end of each cycle by injecting 10 mM Glycine, pH 1.5. Both data processing and kinetic fitting were performed using BIAevaluation software 4.1. Data showing averages of duplicate results (from the same run) is shown in Table 4. The multiple values shown for DMS4031 represent two experiments run on separate occasions. The value of 787 nM probably overestimates the affinity due to the concentrations of ligand analysed

TABLE 4

| | | | | | Top | |
|---|---|---|---|---|---|---|
| | | | | | concen- | # |
| Molecule | Anti- | Ka | Kd | KD | tration | dilu- |
| number | gen | [1/Ms] | [1/s] | [pM] | (nM) | tions |
| DMS4000 | TNFα | 3.65E+05 | 4.16E−05 | 112 | 10 | 6 |
| DMS4000 | VEGF | 9.19E+05 | 4.78E−04 | 520 | 2.5 | 5 |

### Example 3

### Stoichiometry Assessment of Antigen Binding Proteins (Using Biacore™)

This example is prophetic. It provides guidance for carrying out an additional assay in which the antigen binding proteins of the invention can be tested.

Anti-human IgG is immobilised onto a CM5 biosensor chip by primary amine coupling. Antigen binding proteins are captured onto this surface after which a single concentration of TNFα or VEGF is passed over, this concentration is enough to saturate the binding surface and the binding signal observed reached full R-max. Stoichiometries are then calculated using the given formula:

Stoich=Rmax*Mw(ligand)/Mw(analyte)*R(ligand immobilised or captured)

Where the stoichiometries are calculated for more than one analyte binding at the same time, the different antigens are passed over sequentially at the saturating antigen concentration and the stoichometries calculated as above. The work can be carried out on the Biacore 3000, at 25° C. using HBS-EP running buffer.

### Example 4

Design and Construction of CTLA4-Ig Fused to anti-VEGFR2 Adnectin Via a GS Linker (BPC1821)

A codon-optimised DNA sequence encoding CTLA4-Ig (a HindIII site at the N-terminus and BamHI site at the C-terminus were included to facilitate cloning) was constructed and cloned into a mammalian expression vector (pTT expression vector from the National Research Council Canada with a modified multiple cloning site (MCS)) containing the CT01 adnectin. This allowed the adnectin to be fused onto the C-terminus of the CTLA4-Ig via a GS linker. The resulting antigen binding protein was named BPC1821. The DNA and protein sequences of BPC1821 are given in SEQ I.D. No. 26 and 27 respectively.

The expression plasmid encoding BPC1821 was transiently transfected into HEK 293-6E cells (National Research Council Canada) using 293fectin (Invitrogen, 12347019). A tryptone feed was added to the cell culture after 24 hours and the supernatant was harvested after 96 hours. BPC1821 was purified using a Protein A column before being tested in a binding assay.

### Example 5

### VEGFR2 and B7-1 Binding ELISA (BPC1821)

A 96-well high binding plate was coated with 0.4 μg/ml of recombinant human VEGFR2Fc Chimera (R&D Systems, 357-KD-050) in PBS and stored overnight at 4° C. The plate was washed twice with Tris-Buffered Saline with 0.05% of Tween-20. 200 μL of blocking solution (5% BSA in DPBS buffer) was added to each well and the plate was incubated for at least 1 hour at room temperature. Another wash step was then performed. BPC1821 and two negative control antibodies (Sigma I5154 and the bispecific IGF1R—VEGFR2 antigen binding construct BPC1801—heavy chain SEQ ID NO:163 and light chain SEQ ID NO:164) were successively diluted across the plate in blocking solution. After 1 hour incubation, the plate was washed. Recombinant human B7-1 Fc Chimera (RnD Systems, 140-B1-100) was biotinylated using the ECL biotinylation module from GE Healthcare. The labelling was performed at a quarter of the kit recommended level. The biotinylated B7-1 was diluted in blocking solution to 1 μg/mL and 50 μL was added to each well. The plate was incubated for one hour then washed. ExtrAvidin peroxidase (Sigma, E2886) was diluted 1 in 1000 in blocking solution and 50 μL was added to each well. After another wash step, 50 μl of OPD SigmaFast substrate solution was added to each well and the reaction was stopped 15 minutes later by addition of 25 μL of 3M sulphuric acid. Absorbance was read at 490 nm using the VersaMax Tunable Microplate Reader (Molecular Devices) using a basic endpoint protocol.

FIG. 6 shows the results of the ELISA and confirms that bispecific BPC1821 shows binding to both VEGFR2 and B7-1. The negative control antibodies do not show binding to both VEGFR2 and B7-1. Control concentrations were diluted from starting concentrations of 2 μg/ml.

### Example 6

### Design and Construction of CTLA4-Ig Fused to an Anti-VEGF Dab Via a Gs Linker (BPC1825)

The DNA plasmid containing the CTLA4-Ig fused to the anti-VEGFR2 adnectin was used as a base plasmid to construct a CTLA4-Ig-anti-VEGF dAb bispecific. The vector was prepared by digesting the base plasmid with BamHI and EcoRI to remove the adnectin sequence. DNA sequences encoding the anti-VEGF dAb were restricted with BamHI and EcoRI and ligated into the vector. The resulting CTLA4-Ig-anti-VEGF dAb bispecific was named BPC1825, where the dAb was fused onto the C-terminus of the CTLA4-Ig via a GS linker. The DNA and protein sequences of BPC1825 are given in SEQ ID NO:28 and 29, respectively.

The expression plasmid encoding BPC1825 was transiently transfected into HEK 293-6E cells (National Research Council Canada) using 293fectin (Invitrogen, 12347019). A tryptone feed was added to each cell culture after 24 hours and supernatants were harvested after 96 hours. The supernatants were used as the test articles in binding assays.

### Example 7

### VEGF and B7-1 Binding ELISA (BPC1825)

A 96-well high binding plate was coated with 0.4 μg/ml of human VEGF165 (in-house material) in PBS and stored overnight at 4° C. The plate was washed twice with Tris-Buffered Saline with 0.05% of Tween-20. 200 μL of blocking solution (5% BSA in DPBS buffer) was added to each well and the plate was incubated for at least 1 hour at room temperature. Another wash step was then performed. BPC1825 and two negative control antibodies (Sigma I5154 and BPC1824—a CTLA4-Ig-anti-IL-13 dAb fusion—SEQ ID NO:165) were successively diluted across the plate in blocking solution. After 1 hour incubation, the plate was washed. Recombinant human B7-1

Fc Chimera (RnD Systems, 140-B1-100) was biotinylated using the ECL biotinylation module from GE Healthcare. The labelling was performed at a quarter of the kit recommended level. The biotinylated B7-1 was diluted in blocking solution to 1 μg/mL and 50 μL was added to each well. The plate was incubated for one hour then washed. ExtrAvidin peroxidase (Sigma, E2886) was diluted 1 in 1000 in blocking solution and 50 μL was added to each well. After another wash step, 50 μl of OPD SigmaFast substrate solution was added to each well and the reaction was stopped 15 minutes later by addition of 25 μL of 3M sulphuric acid. Absorbance was read at 490 nm using the VersaMax Tunable Microplate Reader (Molecular Devices) using a basic endpoint protocol.

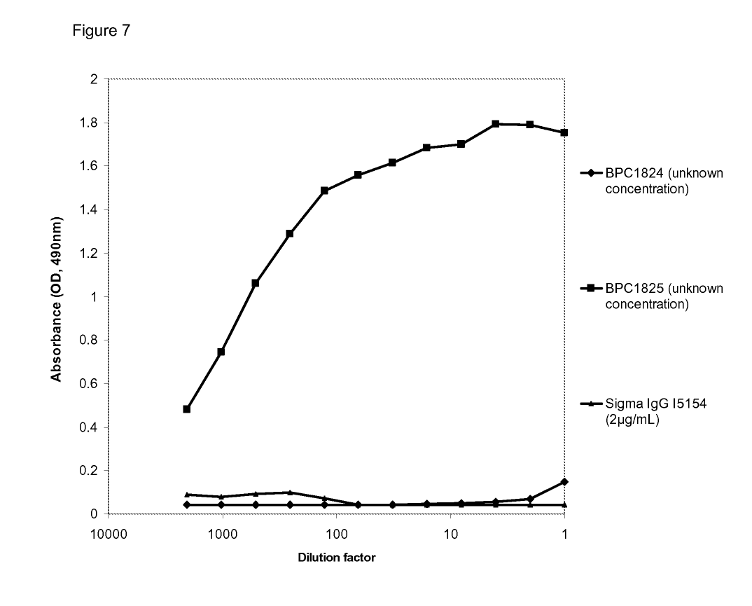

FIG. 7 shows the results of the ELISA and confirms that bispecific BPC1825 shows binding to both VEGF and B7-1. The negative control antibodies do not show binding to both VEGF and B7-1. Concentration of Sigma I5154 IgG was diluted from a starting concentration of 2 μg/ml.

### Example 8

### Design and Construction of a TNFα Receptor Fc Fusion Fused to a VEGF Dab Via an STG or TVAAPPSTG Linker

A codon-optimised DNA sequence encoding a human TNFα receptor Fc fusion (etanercept) was constructed and cloned into a mammalian expression vector (pTT5) along with the DOM15-26-593 anti VEGF dAb from the DMS4000 construct.

The Receptor Fc was flanked with additional sequences to provide an N-terminal Campath1 signal peptide, and provide either an STG linker or TVAAPSTVAAPSTVAAPSTVAAPSTG linker at the C-terminus for fusion to the dAb. The flanking sequences included an AgeI restriction site and a SalI restriction site to facilitate cloning into the vector with the dAb. The resulting antigen binding proteins were named EtanSTG593 and EtanTV4593, respectively. The DNA and protein sequences of EtanSTG593 are given in SEQ ID No:48 and 49, respectively, and of EtanTV4593 are given in SEQ ID No: 50 and 51 respectively.

### Example 9

### EtanSTG593 and EtanTV4593 Purification and VEGF and TNFα Binding Analysis

The EtanSTG593 and EtanTV4593 plasmids were independently expressed in HEK 293-6E cells (National Research Council Canada) using 293Fectin (Invitrogen) for transfection. EtanSTG593 and EtanTV4593 were harvested after 5 days, and purified by MAb Select Sure (GE Healthcare) affinity chromatography to give batch samples M4004 and M4005 respectively. The proteins were formulated in F1 buffer (0.1 M Citrate pH6, 10% PEG300, 5% Sucrose) or ET buffer (10 mM Tris pH7.4, 4% D-Mannitol, 1% Sucrose). The proteins were further purified by Size Exclusion Chromotography on a HiLoad Superdex S200 10/300 GL column (GE Healthcare) to reduce the level of aggregates.

Binding analysis was carried out on a ProteOn XPR36 machine (BioRad™). Protein A was immobilised on a GLM chip by primary amine coupling. The constructs to be tested were captured on this Protein A surface. The analytes, TNFα and VEGF were used at 256 nM, 64 nM, 16 nM, 4 nM and 1 nM. 0 nM (i.e. buffer alone) TNFα and VEGF was used to double reference binding curves.

The novel six by six flowcell set up of the ProteOn allows up to six constructs to be captured at the same time and also allows six concentrations of analyte to be flowed over the captured antibody(s), in all generating 36 interactions per cycle.

To regenerate the Protein A surface, 50 mM NaOH was used, this removed captured construct(s) and allowed another capture and binding cycle to begin. The data obtained was fitted to 1:1 model inherent to the ProteOn analysis software. The run was carried out using HBS-EP as running buffer and at a temperature of 25° C.

TABLE 5
VEGF Binding Results

| Construct | Ka [1/Ms] | Kd [1/s] | KD(nM) |
|---|---|---|---|
| M4004 F1 | 1.18E+05 | 1.01E−04 | 0.850 |
| M4005 F1 | 3.18E+05 | 1.85E−05 | 0.058 |
| M4004 ET | 1.24E+05 | 7.84E−05 | 0.631 |
| M4005 ET | 4.54E+05 | 4.44E−05 | 0.098 |

TABLE 6
TNFα Binding Results

| Construct | Ka [1/Ms] | Kd [1/s] | KD(nM) |
|---|---|---|---|
| M4004 F1 | 5.10E+06 | 1.22E−04 | 0.024 |
| M4005 F1 | 4.95E+06 | 1.05E−04 | 0.021 |
| M4004 ET | 4.81E+06 | 1.15E−04 | 0.024 |
| M4005 ET | 4.87E+06 | 1.38E−04 | 0.028 |

### Example 10

### Prophetic Example

### 10.1 Generating Dual-Targeting Antigen Binding Proteins

A dual-targeting antigen binding construct can be engineered by introducing physical linkages between two previously identified antigen binding proteins e.g. antibody fragments or whole monoclonal antibodies. The physical linkages may be introduced by encoding genetic linker sequences between the two moieties. The nature of the linker in terms of length and amino acid composition may have a bearing on the properties of one or both of the moieties in the bispecific agent. In the event of having multiple antibodies or antibody fragments for generating bispecifics, an empirical approach may be adopted to identify an optimum combination of leads.

Individual binding moieties such as mAbs, FAbs, ScFvs, dAbs etc. against defined targets can be identified and developed in isolation using a variety of well documented in vivo (for example: Harlow, E and Lane, D (1998) Antibodies, A Laboratory Manual, Cold Spring Harbor Laboratory Press) and in vitro (for example: Barbas III, C F et al (2001) Phage Display, A Laboratory Manual, Cold Spring Harbor Laboratory Press) techniques to deliver agents with known properties of potency, efficacy and biophysical behaviour. From these individual agents a number of different bispecific opportunities arise which are only limited by the degree of complexity of the molecular engineering required to create them. The desired molecular architecture is normally determined by the nature of the condition to be treated. For example, for chronic dosing a molecular format that delivers an intrinsically long in vivo half life is to be favoured. This can be most readily achieved by the inclusion of the Fc region of an IgG antibody which delivers long terminal half life by virtue of salvage recycling pathways. Thus a mAb or other Fc-based bispecific is a frequently employed format.

To develop a mAb-based dual targeting molecule one potential approach is to append an antibody fragment to a full IgG. At a molecular level, this can be done by introducing a restriction site at one of the termini of the mAb chain and inserting an antibody fragment such that the mAb chain is extended with an additional functional unit. The nature of the linker between the functional units may need to be varied to optimise the overall properties of the bispecific. If a range of different antibody fragments are available that address the same target, these may be directly compared with one another using this approach. Bispecifics of this nature will normally be expressed in mammalian cells, typically HEK293 cells transiently but CHO cells for stable cells lines and large-scale manufacturing. For TNF/VEGF bispecifics, an anti-TNFα mAb may be linked to a VEGF binding protein such as an antibody fragment in this manner, or alternatively an anti-VEGF mAb may be linked to an anti-TNFα binding protein. For example, TNFα and VEGF antagonists that may be utilised in this way are listed in table 1 and 2, respectively. In such an exercise, if all possible reagents are available, all potential combinations would be tested.

Non mAb-based bispecifics can be made by linking two antibody fragments or other proteins which bind antigens in a generally analogous manner together as a genetic fusion. The junction of the two units is normally represented by a linker of a length and sequence composition that may be determined empirically. Such molecules allow freedom of molecular engineering due to their modular, single chain nature and afford the possibility of expression in systems other than mammalian cells.

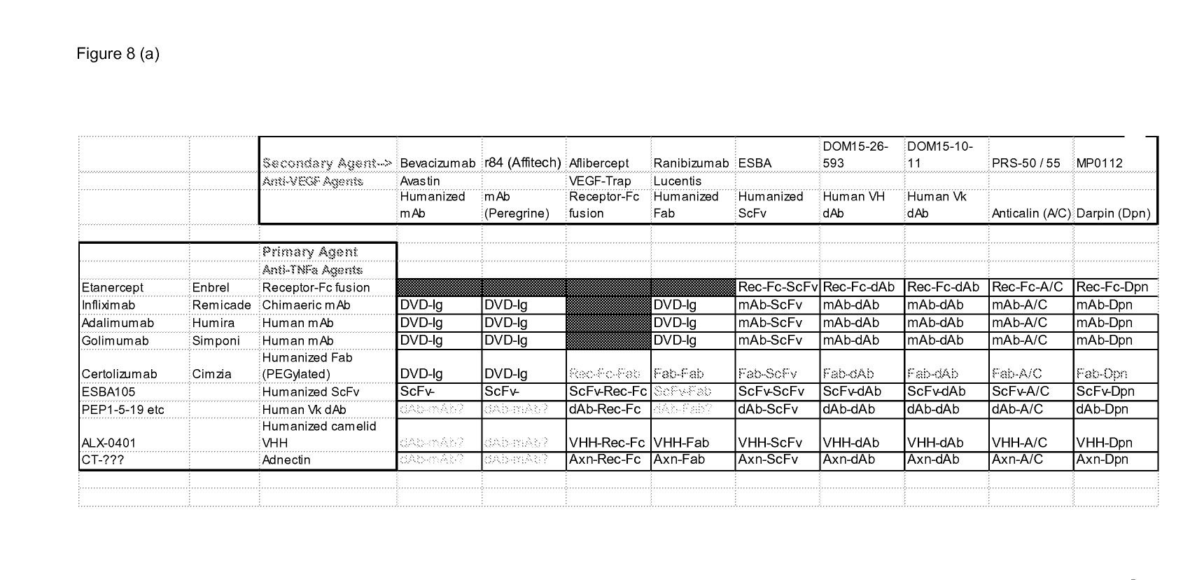

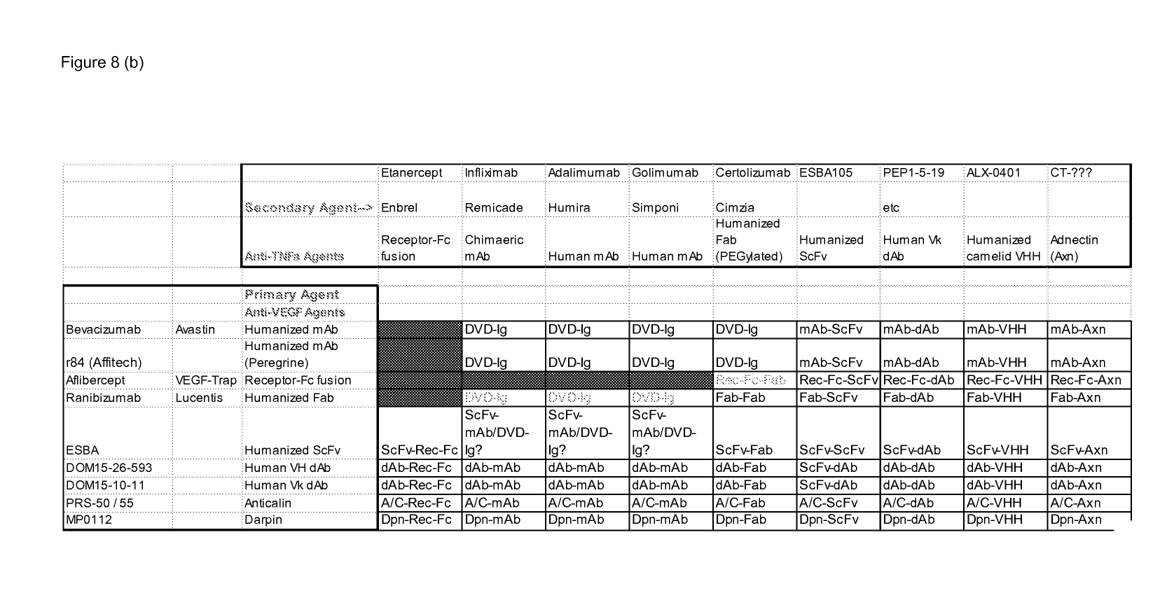

FIG. 8 shows a matrix of possible dual targeting constructs that may be used in accordance with the invention. Sequences of a number of the possible dual targeting constructs shown in FIG. 8 are given in SEQ ID NO:73-140. In these specific dual targeting molecules a ‘TVAAPS’ linker (SEQ ID NO:4) is used to link the component parts, with the exception of heavy chains in DVD-Igs, DVD-Fabs fusions with N-terminal ScFvs (SEQ ID NO:116-118) and fusions with N-terminal VH dAbs (SEQ ID NO:133, 134) where the linker is ‘ASTKGPS’ (SEQ ID NO:6). SEQ ID NO:73-140 are exemplary only and the skilled person would realise that other linkers and constructions are possible.

TABLE 7
abbreviations used in FIG. 8

| | |
|---|---|
| IgG | Immunoglobulin G |
| mAb | Monoclonal antibody |
| FAb | Fragment for antigen binding |
| ScFv | Single chain variable fragment |
| dAb | Domain antibody |
| VHH | Camelid single domain antibody |
| A/C | Anticalin |
| Dpn | Darpin |
| Axn | Adnectin |
| DVD-Ig | Dual variable domain IgG |
| Fc | IgG CH2-CH3 region |
| Rec | Receptor |
| PEG | Polyethylene glycol |

### 10.2 Testing the Dual-Targeting Antigen Binding Proteins for Required Characteristics

Potency/Affinity: A fundamental property of a bispecific molecule suitable for further development is a kinetic binding affinity (usually determined by a form of surface plasmon resonance (SPR), for example BIAcore) for antigen which, in turn, would be used to predict a minimum pharmacologically effective concentration after a given therapeutic dose based upon prior knowledge of antigen concentration and availability. The affinity may also be predicted to be related to neutralisation potency, an attribute normally assessed by an in vitro assay that determines the concentration of compound that mediates a particular pharmacological effect. This may be the inhibition of a receptor/ligand binding event or the stimulation/inhibition of a downstream response pathway. For example, the potency of a TNF antagonist may be assessed by the extent to which it prevents the production of other cytokines that are regulated by TNF. A common form of this would be the reduction in the secretion of IL8 from MRC-5 cells in response to TNF. For a VEGF antagonist, the extent to which receptor phosphorylation is reduced is a direct consequence of the inhibitory potency of anti-VEGF agent, whilst the reduction in proliferation of HUVEC cells is a biological correlate of this effect. As with the kinetic affinity, the bispecific would be required to demonstrate target potency for both antigens.

Biophysics: Because conventional mAbs are known to have good expression, biophysical and pharmacokinetic profiles, any developable bispecific molecule would be required to demonstrate similar characteristics. Expression level would be determined during transient and stable cell culture and would be required to be in the same normal range as conventional therapeutic antibodies. The bispecific would need to be amenable to similar purification processes to mAbs (for example protein-A capture) and other down stream processing (DSP) steps that are required in the production of clinical grade material. The purified protein would need to demonstrate a clean, symmetrical size exclusion chromatography (SEC) profile, stability at high (>25 mg/ml) protein concentrations in biocompatible buffers and resistance to a range of stress conditions (temperature, pH, freeze-thaw, deamidation conditions etc).

Pharmacokinetics (PK)/Pharmacodynamics (PD): The pharmacokinetic profile of a bispecific antigen binding protein is required to be consistent with the nature of the targets and the disease setting. In the majority of cases, antibodies are positively differentiated by virtue of their long serum half life and this is usually the desired profile. PK as normally assessed in both rodent and primate species and the terminal half life (t_{1/2}β) of the bispecific should be comparable with that of antibody agents against the same targets (it is assumed that the bispecific will reflect the more rapidly cleared species in the event of the two activities being metabolised at radically different rates). PK assays for bispecific molecules ideally measure the two activities in a single assay (a bridging assay), thereby providing confidence that the residual drug in the circulation is intact and fully bifunctional (for example, TNF is immobilized on a plate, the samples containing drug are added to the plate and the amount of bispecific present assayed by the addition of, for example, biotinylated VEGF which is itself detected by an anti-biotin agent). Other in vivo analyses on bispecific compounds would include the testing in models of disease under the proviso that such models exist and that the cross-reactivity of the bispecific with the host species is well understood. For a TNF/VEGF bispecific this may include inflammatory conditions where the inflammation is exacerbated by increased vascular leakage or a vascular proliferative condition where the activation of macrophages in the local environment exacerbates the disease state. In primates, such models may also allow the derivation of certain pharmacodynamic markers of activity that may play a role in the calculation of dose etc.

Safety: The relative novelty of bispecific formats (even if the component parts and targets are precedented) raises issues of safety and tolerability. As with any biological drug, the full range of toxicology tests would be required, with an increased emphasis on any hypothetical concerns related to the bispecific molecular format. This may include additional unanticipated pharmacology or the potential for increased immunogenicity. The latter possibility may be addressed using in silico tools to look for T-cell epitopes which could be used to construct a risk profile for this aspect of the molecule.

Non mAb-based bispecific formats (for example direct fusion of two antibody or antibody-like fragments) can be judged on many of the same criteria of affinity, potency and biophysical behaviour, although some attributes, in particular PK, may vary with different molecular format. Such molecules may also be produced in different expression systems (for example, in prokaryotic cells), which may in itself create different requirements especially with regards to purification, DSP and safety studies.

### Example 11

TNF/VEGF dAb-dAb In-Line Fusions (ILF)

Detailed below is a method for constructing dAb-dAb in-line fusions in order to make a TNF-VEGF bispecific. However, as described above in Example 10, the same approach could be used to generate any other bispecific based upon antibodies or antibody fragments with similar target specificities.

Bispecific molecules that have the potential to inhibit both TNFα and VEGF were constructed by the genetic fusion of two single Domain Antibodies (dAbs) into a dAb-dAb in-line fusion (ILF). To construct these molecules, independently selected dAbs against the two targets were isolated by phage display and high affinity and potency against the targets was achieved by rounds of affinity maturation using a range of suitable techniques. The final molecules that were selected for the ILFs were DOM15-26-593 (anti-VEGF) (SEQ ID NO:1) and PEP1-5-19 (anti-TNFα) (SEQ ID NO:35).

DOM15-26-593 is a VH dAb with a monomeric affinity for human VEGF-A of approximately 1 nM. PEP1-5-19 is a Vk dAb with a monomeric affinity for human TNFα of approximately 8 nM. Two different ILF constructs were made, one with the DOM15-26-593 dAb at the amino terminus (abbreviated below as “DOM-PEP”), and one with the PEP1-5-19 dAb in this location (“PEP-DOM”). The two dAbs in the ILFs were separated by a short linker that was derived from a sequence naturally associated with the C terminus of a VH or a Vk dAb. Hence the ILF with the VH dAb at the N-terminus included the linker “ASTKGPS” (SEQ ID NO:6—the natural extension from VH into CH1), and the ILF with the PEP1-5-19 at the N-terminus included the linker sequence “TVAAPS” (SEQ ID NO:4—the natural extension from Vk into Ck).

To make the ILFs, the mammalian transient expression vector pTT5 (NRC, Canada) was modified to include a secretion signal and appropriate cloning sites. These were as detailed below in table 8. To make the DOM-PEP construct, individual fragments corresponding to the DOM15-26-593 dAb and PEP1-5-19 domain dAb were amplified with the respective gene specific primers as described below. Linker sequences and restriction sites were incorporated within the primer sequences.

TABLE 8

| Primer | Sequence 5′-3′ | Comments |
|---|---|---|
| AVG18 | attatgggatccaccggcgaggtgcagctgttggtgt | forward primer for |
| | (SEQ ID NO: 52) | DOM15-26-593 |
| | | (DOM-PEP, has |
| | | BamHI site) |
| AVG19 | gctggggcccttggtgctagcgctcgagacggtgaccagg | reverse primer for |
| | (SEQ ID NO: 53) | DOM15-26-593 |
| | | (DOM-PEP, has |
| | | NheI site) |
| AVG26 | ctcgagcgctagcaccaagggccccagcgacatccagatgaccc | forward primer for |
| | (SEQ ID NO: 54) | PEP (DOM-PEP, |
| | | has NheI site) |
| AVG21 | ttatgtcaagcttttaccgtttgatttccaccttggt | reverse primer for |
| | (SEQ ID NO: 55) | PEP (DOM-PEP, |
| | | has HindIII site) |
| AVG22 | attatgggatccaccggcgacatccagatgacccagtctcc | forward primer for |
| | (SEQ ID NO: 56) | PEP (PEP-DOM, |
| | | has BamHI site) |
| AVG36 | gcgccgccaccgtacgtttgatttccaccttggtccc | reverse primer for |
| | (SEQ ID NO: 57) | PEP (PEP-DOM, |
| | | has BsiWI site) |
| AVG37 | caaacgtacggtggcggcgccgagcgaggtgcagctgttggtgtc | forward primer for |
| | (SEQ ID NO: 58) | DOM15-26-593 |
| | | (PEP-DOM, has |
| | | BsiWI site but short |
| | | overhang for digest) |
| AVG25 | ttatgtcaagcttttagctcgagacggtgaccag | reverse primer for |
| | (SEQ ID NO: 59) | DOM15-26-593 |
| | | (PEP-DOM, has |
| | | HindIII site) |
| AVG24 | ggtggaaatcaaacgtacggtggcggcgccgagcga | forward primer for |
| | (SEQ ID NO: 60) | DOM15-26-593 |
| | | (PEP-DOM, has |
| | | BsiWI site |
| | | appropriate |
| | | overhang for |
| | | subsequent digest) |

N.B. restriction sites are underlined in DNA sequences

DOM15-26-593 for the DOM-PEP construct was amplified with AVG18 and AVG19 and PEP1-5-19 for the DOM-PEP construct was amplified with AVG26 and AVG21. After purification the PCR fragments were digested with BamHI and NheI, and NheI and HindIII respectively and the fragments purified. They were then added to a 3-fragment ligation with a modified form of the vector pTT5 which contained a multiple cloning site that allowed the insertion of a BamHI-HindIII fragment downstream of a eukaryotic promoter. Ligations, transformations and analysis of resulting colonies was done using standard techniques, with nucleotide sequence analysis confirming that the resulting vector contained an insert with a sequence as laid out in SEQ ID NO:61, predicting a translation product shown in SEQ ID NO:62.

For the PEP-DOM construct, the PEP1-5-19 dAb was amplified with AVG22 and AVG36 and the DOM15-26-593 dAb with AVG37 & AVG25. These fragments were digested with BamHI and BsiWI (PEP) and BsiWI and HindIII (DOM), respectively. The DOM fragment was found to digest poorly and this was attributed to the short overhang on the 5′ end of the primer. The PCR product was therefore re-amplified with AVG25 and AVG24 to extend the overhang, the digest was repeated and the fragment added to a 3-fragment ligation along with digested PEP insert and the pTT5 vector as described above. Ligations, transformations and analysis of resulting colonies was done using standard techniques, with nucleotide sequence analysis confirming that the resulting vector contained an insert with a sequence as laid out in SEQ ID NO:63, predicting a translation product shown in SEQ ID NO:64.

The sequenced clones were prepared for transfection by DNA maxiprep and DNA transfected into HEK293-6E cells (National Research Council Canada) using standard methodology. After clarification of the culture medium, the recombinant protein was harvested from transfected cell supernatant by protein-A affinity chromatography and purified material buffer exchanged into PBS and quantified. The ability of these proteins to bind both TNFα and VEGF was then assessed by surface plasmon resonance (SPR) as described below.

Using a number of monoclonal antibodies (alternatively protein A or protein L could be used) believed to bind to either VH or Vk dAbs away from the dAb CDR regions, the DOM-PEP and PEP-DOM proteins were captured on the sensor surface via the mAbs, the TNF and VEGF ligands were flowed over the captured bispecific and the binding characteristics analysed. The analysis determined that when the compounds were captured with either one of 2 different anti-Vk dAbs tested the binding of the TNF ligand was impaired, suggesting that this capture antibody was sterically interfering with the ligand binding. Further analysis was therefore restricted to the bispecific captured with an anti-VH dAb monoclonal antibody.

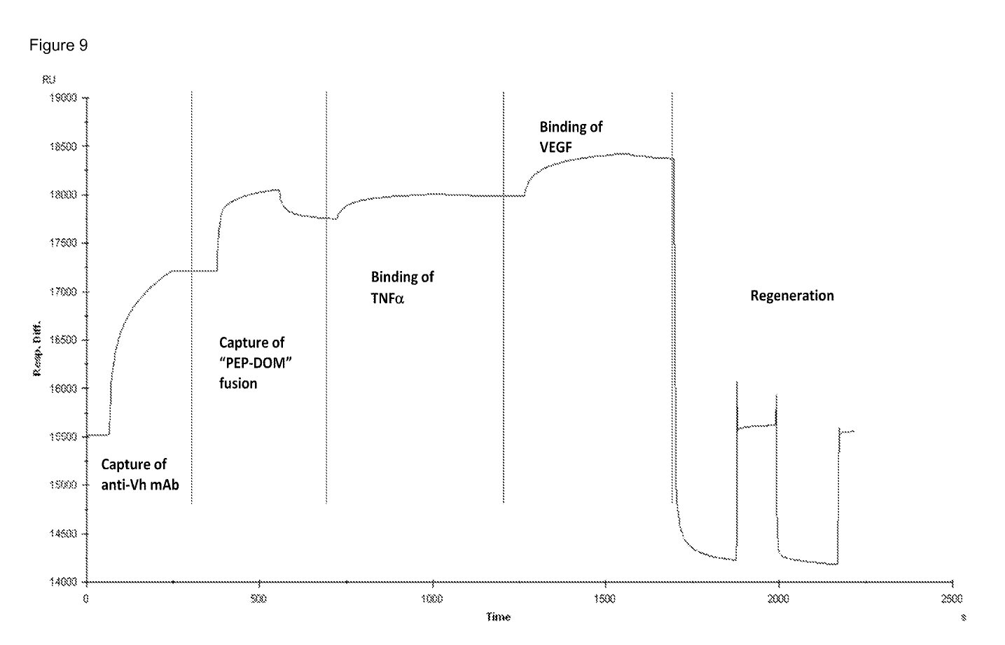

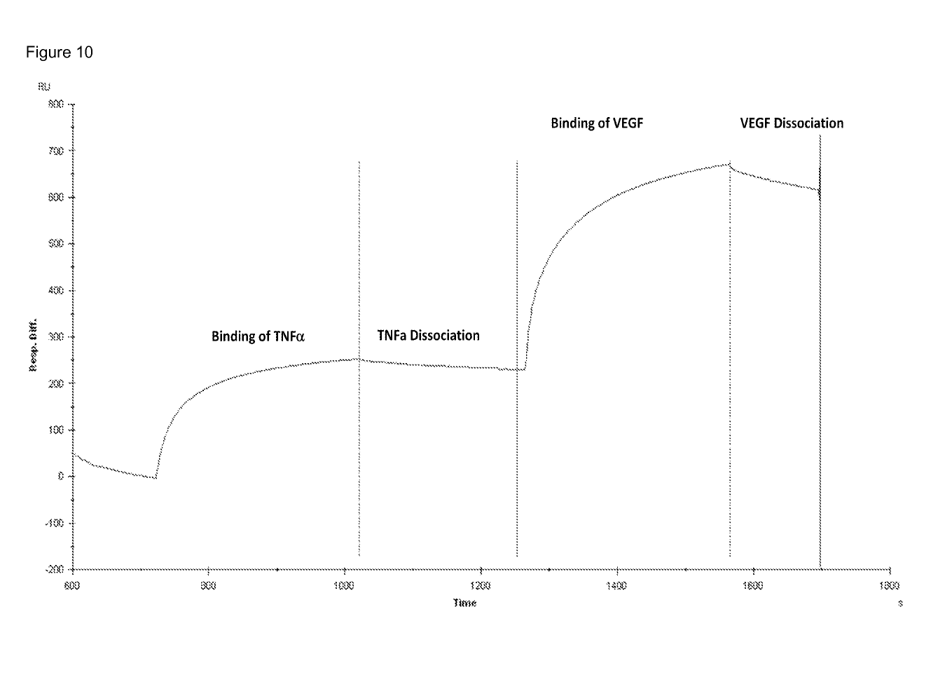

Approximately 1600 response units (RUs) of the anti-VH monoclonal were captured on a protein-A surface and the test compounds passed over the complex. The experimental set up was designed to provide a qualitative rather than quantitative measure of the binding activities therefore estimations of kinetics etc. were not possible. The clearest data was obtained for the PEP-DOM protein, where the two dAbs were both clearly able to bind to the ligands independently and simultaneously as evidenced by the additive binding curves (FIGS. 9 & 10).

Closer analysis of the binding events in the curve in FIG. 9 demonstrates the binding of both ligands to the PEP-DOM protein.

The possibility of DOM-PEP binding both TNFα and VEGF is also seen (data not shown).

### Example 12

### An In Vivo Study: Laser-Induced Choroidal Neovascularisation (CNV) in Rats: Testing DMS1571 (VEGF-dab) and Enbrel™ Separately

### Rationale

Results obtained in a previous experiment showed that the anti-VEGF antagonist, DMS1571 (an Fc formatted version of the DOM15-26-593 anti-VEGF dAb, which exists as a dimer of SEQ ID NO:65), is efficacious in the rat laser-induced choroidal neovascularization (CNV) model. The aim of this experiment was to further evaluate the dose-ranging of this molecule in the rat CNV model and, in addition, to undertake a dose ranging study of a TNFα antagonist (Enbrel™) in the same model. DMS4000 was also tested in this the study.

### Methodology

### Animals

12-week old Dark Agouti (DA) rats (Harlon Olac) were used in these studies. Prior to procedures animals were surgically anesthetized by intraperitoneal injection of a mixture of Ketamine (37.5%, Dodge Animal Health Ltd.), Dormitor (25%, Pfizer Animal Health, Kent) and sterile water (Pfizer Animal Health, Exton, Pa.) at 0.175 ml/100 g and pupils were dilated with a combination of topical 1% tropicamide (Alcon Laboratories, Fort Worth, Tex.) and 2.5% phenylephrine (Akorn, Inc., Decatur, Ill.). All animal experiments conformed to the ARVO Statement on the Use of Animals in Ophthalmic and Vision Research.

### Experimental CNV

Experimental CNV was induced unilaterally in groups of 2-4 month old female DA rats by rupturing Bruch's membrane using laser light photocoagulation (PC). Dye laser PC was performed using a diode-pumped, 532 nm argon laser (Novus Omni Coherent Inc., Santa Clara, Calif.) attached to a slit lamp funduscope, and a handheld planoconcave contact lens (Moorfields Eye Hospital, London, UK) applied to the cornea to neutralize ocular power. Eight lesions (532 nm, 150 mW, 0.15 second, 100 μm diameter) were made in a peripapillary distributed and standardized fashion centered on the optic nerve at 500 μm radius (at 1-1.5 mm from optic disc) and avoiding major vessels in each eye. The morphologic end point of the laser injury was identified as the temporary appearance of a cavitation bubble, a sign associated with the disruption of Bruch's membrane (for background reference, general methods are disclosed in Campos, Amaral, Becerra, & Fariss, 2006 A novel imaging technique for experimental choroidal neovascularization. Invest Ophthalmol V is Sci, 47(12), 5163-5170, which is herein incorporated by reference in its entirety). Laser spots that did not result in the formation of a cavitation bubble were excluded from the studies.

### In Vivo Imaging

In vivo image data of CNV and associated leakage was generated using confocal high-resolution Scanning Laser Ophthalmoscope (SLO) Fluorescein Angiography (FA) (0.3 ml 5% intra-abdominally injected Fluorescein Sodium, FS obtained from Moorfields Eye Hospital, London, UK) at 7 days after lesion generation followed by a second imaging session 14 days post-procedure. Time points were chosen based on previous historical control studies on the time course of changes in intensity and area of fluorescein staining in angiograms taken after laser PC in non-treated rats. These historical studies showed that fluorescein staining was first observed 4 days after PC and that the intensity of the staining then rapidly increased reaching its peak approximately 14 days after photocoagulation (for general background on methodology see Kamizuru et al., 2001; Monoclonal antibody-mediated drug targeting to choroidal neovascularization in the rat. Invest Ophthalmol V is Sci, 42(11), 2664-2672; Takehana et al., 1999 Suppression of laser-induced choroidal neovascularization by oral tranilast in the rat. Invest Ophthalmol V is Sci, 40(2), 459-466, which are herein incorporated by reference in their entirety). Further assessment was not undertaken as the time course of experimental CNV in these studies indicated that fluorescein leakage begins to decrease approximately 5 weeks after photocoagulation. Baseline reflectance (at 488 nm and 790 nm) and autofluorescence (ex. 488 nm, em. >498 nm) images were made prior to injection of FS to help locate lesions in FA images. The arterio-venous phase was recorded immediately after FS injection. Fluorescein angiograms were thereafter recorded one minute after injection and again four minutes after injection, the latter 4 min data sets being used for statistical analysis.

### Evaluation and Statistical Analysis of Image Data

The effect of drug treatment was evaluated by quantitative assessment of late-phase (4±1 minutes after FS injection) fluorescein angiography. Leakage was defined as the presence of hyperfluorescent areas corresponding with lesions in reflectance images. Prior to quantification the gain and brightness of all images used in analysis were normalized. The intensity and area of leakage in late-phase fluorescein angiography was quantified by multiplying the diameter of leakage (μm) with the mean pixel brightness value (0 to 1) in that area. Unpaired t-tests were used to compare results between test groups. Values of P<0.05 were considered statistically significant. Data are shown as means±SEM unless otherwise noted. Before image analysis was performed identification was scrambled and quantification was undertaken in masked fashion.

immunohistochemical Detection of Macrophages in Rat CNV Lesions

Eyes which had previously been subject to fluorescence angiography in CNV studies were immediately enucleated and fixed in 4% p-formaldehyde. The eye-cup was then prepared from the treated eye of each animal and flat-mounted following four butterfly incisions. The macrophage content of vascular lesions determined by immunohistochemical staining using ED1 mAb and subsequently quantitated by counting ED1 positive cells—ED1 (CD68) mAb (catalogue number MCA341 Serotech, Kidlington, Oxford, UK)

### Treatments

The table below (table 9) shows the treatments given to each experimental group

| | | Total | Concentration | | |
|---|---|---|---|---|---|
| Number | Compound | Dose μg | mg/ml | Volume μl | Administration |
| 1 | Vehicle A—50 mM | N/A | N/A | 2 | intravitreal |
| | NaAcetate pH 5.5, | | | | |
| | 104 mM NaCl, 0.025% | | | | |
| | Tween 80 | | | | |
| 2 | DMS1571 in vehicle A | 2 | 1 | 2 | Intravitreal |
| 3 | DMS1571 in vehicle A | 1 | 0.5 | 2 | Intravitreal |
| 4 | DMS1571 in vehicle A | 0.5 | 0.25 | 2 | Intravitreal |
| 5 | DMS1571 in vehicle A | 0.2 | 0.1 | 2 | Intravitreal |
| 6 | DMS1571 in vehicle A | 0.1 | 0.05 | 2 | intravitreal |
| 7 | Vehicle B—4% Mannitol, | N/A | N/A | 2 | Intravitreal |
| | 1% sucrose, 10 mM | | | | |
| | TrisHCL pH 7.4 | | | | |
| 8 | Enbrel ™ in vehicle B | 30 | 15 | 2 | Intravitreal |
| 9 | Enbrel ™ in vehicle B | 10 | 5 | 2 | Intravitreal |
| 10 | Enbrel ™ in vehicle B | 3 | 1.5 | 2 | Intravitreal |
| 11 | Enbrel ™ in vehicle B | 1 | 0.5 | 2 | Intravitreal |
| 12 | Enbrel ™ in vehicle B | 0.3 | 0.15 | 2 | Intravitreal |
| 13 | DMS4000 in vehicle C | 2 | 1 | 2 | Intravitreal |
| 14 | Vehicle C—100 mM | N/A | N/A | 2 | intravitreal |
| | NaCitrate pH 6, 10% | | | | |
| | PEG300, 5% sucrose | | | | |

In each case, compounds were administered by intravitreal injection immediately prior to laser PC.

### Results of Laser-Induced CNV Studies

High-magnification fluorescein angiography was performed at two time points, at 7 days and 14 days after PC, on the treated eyes. Images were graded for choroidal leakage associated with experimental CNV and other vascular abnormalities related to the treatment noted. Images were recorded in both near-infrared reflectance (IR) and auto-fluorescence mode (AF). IR images were used to locate lesions in the retina prior to injecting the fluorescein contrast agent. All images were recorded at the level of the RPE (retinal pigment epithelium).

### Effect of DMS1571 (VEGF-Dab) and Enbrel™ in Rat CNV

TABLE 10
DMS1571

| | 1.0 | 2.0 | 3.0 | 4.0 | 5.0 | 6.0 |
|---|---|---|---|---|---|---|
| mean₀₇ | 49.7 | 38.9 | 37.5 | 43.1 | 53.3 | 55.8 |
| mean₁₄ | 53.0 | 36.7 | 43.5 | 39.6 | 49.1 | 48.9 |
| | 1 | 2 | 3 | 4 | 5 | 6 |
| SEM₀₇ | 2.189 | 1.623 | 1.738 | 2.877 | 2.761 | 2.952 |
| SEM₁₄ | 1.967 | 1.613 | 3.054 | 1.834 | 2.503 | 4.389 |

Mean+/−SEM for CNV leakage assessed at 7 and 14 days for DMS1571 1.0-vehicle, 2.0-2 μg DMS1571, 3.0-1 μg DMS1571, 4.0-0.5 μg DMS1571, 5.0-0.2 μg DMS1571, 6.0-0.1 μg DMS1571. Agents were injected immediately prior to induction of laser injury. N=5 animals per group in all cases. All compounds were administered by intravitreal injection in a volume of 2 μl.

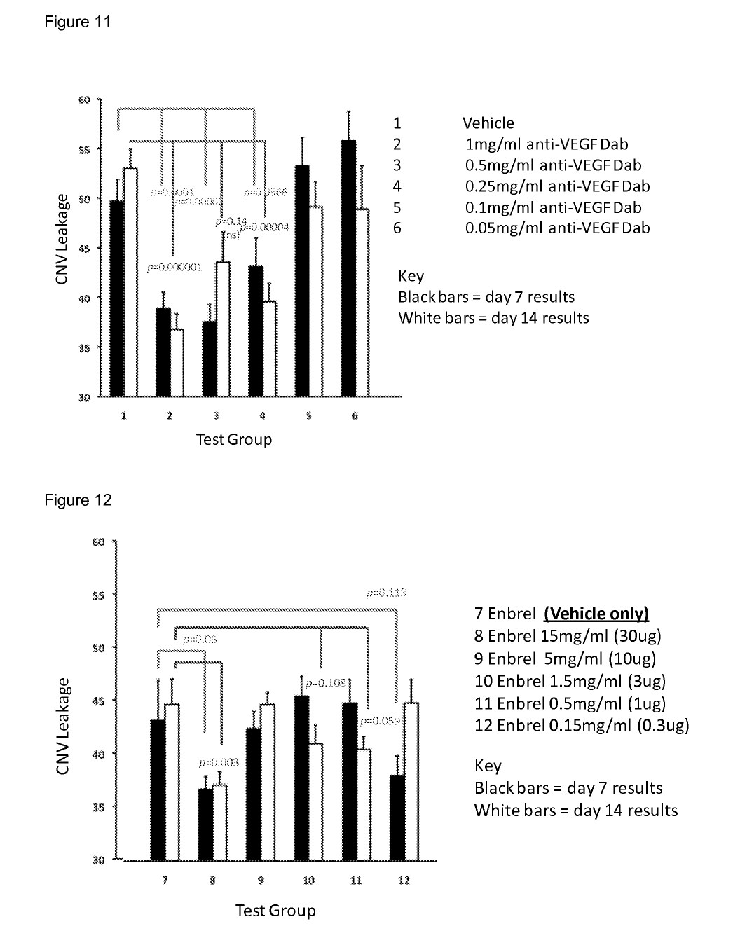

FIG. 11 is a graphical representation of data presented in Table 10. All compounds were administered by intravitreal injection in a volume of 2 μl. Black bars represent day 7 results. White bars represent day 14 results.

TABLE 11
Enbrel ™

| | 7.0 | 8.0 | 9.0 | 10.0 | 11.0 | 12.0 |
|---|---|---|---|---|---|---|
| mean₀₇ | 43.7 | 37.0 | 42.9 | 46.0 | 45.3 | 38.3 |
| mean₁₄ | 45.2 | 37.4 | 45.2 | 41.4 | 40.8 | 45.4 |
| | 7 | 8 | 9 | 10 | 11 | 12 |
| SEM₀₇ | 3.934 | 1.247 | 1.649 | 1.912 | 2.294 | 1.917 |
| SEM₁₄ | 2.469 | 1.302 | 1.167 | 1.794 | 1.246 | 2.268 |

Mean+/−SEM for CNV leakage assessed at 7 and 14 days for test Enbrel™ 7.0-vehicle, 8.0-30 μg Enbrel™, 9.0-10 ug Enbrel™I, 100-3 μg enbrel. 11.0-1 μg Enbrel™, 12.0-0.3 μg Enbrel™. Agents were injected immediately prior to induction of laser injury. N=5 animals per group in all cases. All compounds were administered by intravitreal injection in a volume of 2 μl.

FIG. 12 is a graphical representation of data presented in Table 11. All compounds were administered by intravitreal injection in a volume of 2 μl. Black bars represent day 7 results. White bars represent day 14 results.

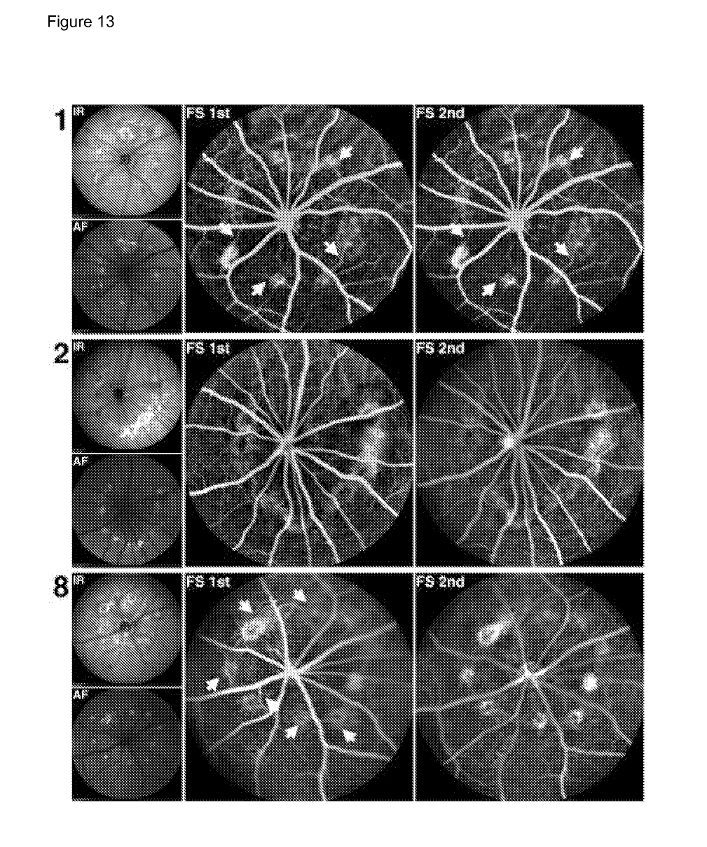

FIG. 13 shows infrared (IR, upper left panel), autofluorescence (AF, lower left panel) and fluorescien angiography (FS, large panel) at 7 days (FS 1st) and 14 days (FS 2nd) after laser PC—showing example images. 1. Vehicle treated eyes, 2. eyes treated with 2 μg DMS1571 and 8. eyes treated with 30 μg Enbrel™. It is notable that the CNV lesions appear more punctuate and less diffuse than lesions responding to treatment with DMS1571. Arrows indicate neovascularisations indicated in both control and Enbrel™ treated animals but not in DMS1571 animals.

TABLE 12
DMS4000

| | 13.0 | 14.0 |
|---|---|---|
| mean₀₇ | 33.4 | 36.8 |
| mean₁₄ | 35.7 | 42.5 |
| | 13 | 14 |
| SEM₀₇ | 1.888 | 2.559 |
| SEM₁₄ | 1.241 | 2.131 |

Mean+/−SEM for CNV leakage assessed at 7 and 14 days for DMS4000 13.0-2 μg DMS4000, 14.0-vehicle, Agents were injected immediately prior to induction of laser injury. N=5 animals per group in all cases. All compounds were administered by intravitreal injection in a volume of 2 μl.

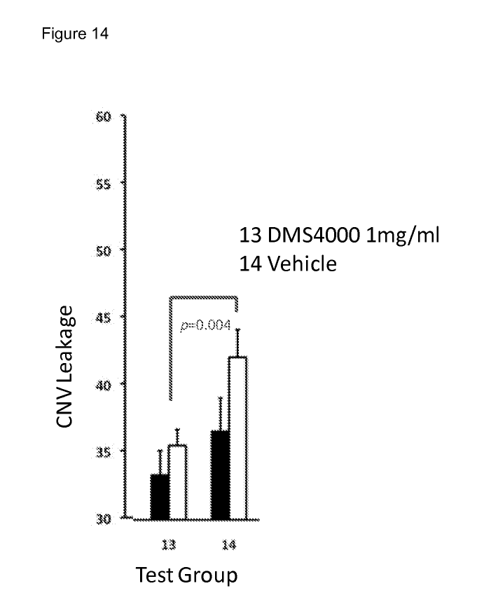

FIG. 14 is a graphical representation of data presented in Table 12. All compounds were administered by intravitreal injection in a volume of 2 μl.

### Effect of DMS1571 (VEGF-dab) and Enbrel™ on Macrophage Content of Rat CNV Lesions

TABLE 13
Quantitation of ED1 positive cells (macrophages) in CNV lesions

| Group | Macrophage (ED1 positive) content of CNV lesions | |
|---|---|---|
| Vehicle (group 1) | 35.2 (mean) | 5.9 (SEM) |
| DMS1571 (group 2) | 29.3 | 4.1 |
| Enbrel (group 8) | 16.2* | 1.37 |

*p < 0.0016 vs control, n = 5 eyes in each case

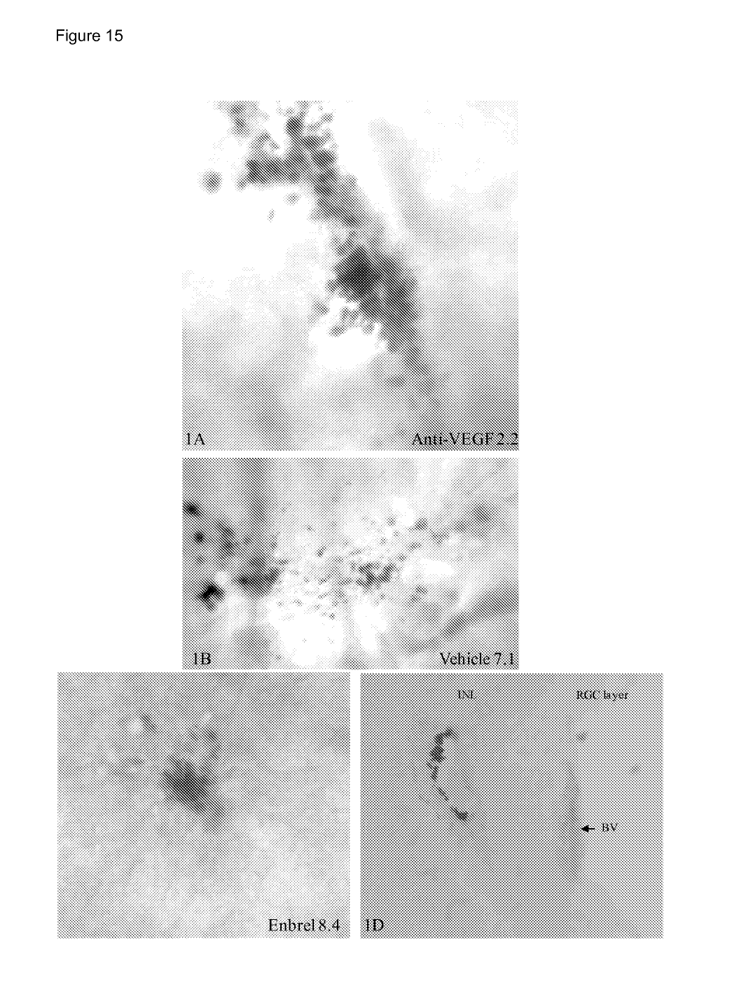

FIG. 15 shows example photomicrographs of flat-mounted retinae stained with ED1 mab. Panels 1A-1B and panel Enbrel 8.4 show flat-mounts of retinas from eyes treated with anti-VEGF (DMS1571) (1A), Vehicle only (1B) or Enbrel (Enbrel 8.4). Macrophages, associated with laser burn site, visualised with ED1 (CD 68, black) X20. Panel 1D shows a Cryostat section (20 μm) of retina showing macrophages (ED1+, black) associated with laser burn site which has penetrated to the inner nuclear layer (INL) of the retina. RGC, retinal ganglion cell layer; BV, blood vessel. x20.

### CONCLUSIONS

The results illustrate that DMS1571 is effective in significantly attenuating CNV disease. The strong and robust effect is noted at doses above 1 μg with the 0.5 μg dose showing a sub-maximal effect and at doses less than 0.5 μg the therapeutic is ineffective. The experiments show that doses at 30 μg of Enbrel™ are also effective in the model and lower doses ineffective. The finding that both VEGF inhibitors, as exemplified by DMS1571, and inhibitors of TNFα, as exemplified by Enbrel™, are able to independently attenuate choroidal neovascular disease in a rodent model suggests that a single therapeutic entity comprising both VEGF and TNFα capabilities, as exemplified by DMS4000, would be useful in the treatment of choroidal neovascular AMD. It is observed that DMS4000 (in which the TNFα binding function is not compatible with binding rat TNFalpha) performs equally well in the rat CNV model as DMS1571 at an equivalent dose.

It is notable from the fluorescence angiography pictures when comparing the DMS1571 treated eyes with the Enbrel™ treated eyes that the Enbrel™ eyes have a distinctive patterning in which the lesions appear more punctuate and less diffuse when compared to DMS1571 treated eyes. These differences in lesion patterning are highly suggestive of independent mechanisms of action of the DMS1571 (VEGF antagonist) and Enbrel™ (TNFα antagonist) therapeutics. This assertion is further supported by the finding that in the Enbrel™ treated group significantly fewer macrophages are recuitred to the CNV vascular lesions.

### Example 13

### An In Vivo Study: Laser-Induced Choroidal Neovascularisation (CNV) in Rats: Testing DMS1571 (VEGF-dab) and Enbrel™ in Combination

The methods used in this example were essentially the same as those given in Example 12.

### Table 14 Below Shows the Treatments Given to Each Experimental Group.

| | | Total Dose | Concentration | Total Volume | |
|---|---|---|---|---|---|
| Identification | Compound | μg | mg/ml | μl | Administration |
| A | DMS1571 | 2 | 1 | 2 | intravitreal |
| B | DMS1571 | 2 DMS1571 | 2 DMS1571 | 2^{#} | intravitreal |
| | plus | 30 | 30 Enbrel ™ | | |
| | Enbrel ™ | Enbrel ™ | | | |
| C | Vehicle* | N/A | N/A | 2 | intravitreal |
| D | DMS1571 | 0.5 | 0.25 | 2 | intravitreal |
| | | DMS1571 | | | |
| E | DMS1571 | 0.5 | 0.5 DMS1571 | 2^{#} | intravitreal |
| | plus | DMS1571 | 30 Enbrel ™ | | |
| | Enbrel ™ | 30 | | | |
| | | Enbrel ™ | | | |

*Vehicle—50 mM NaAcetate 10 mM TrisHCL pH 7.4, 104 mM NaCl, 0.025% Tween 80, 4% mannitol, 1% sucrose
^{#}in cases where both DMS1571 and Enbrel ™ are being administered together, 1 μl of each is administered

TABLE 15
Effect of DMS1571 (VEGF-dab) and Enbrel ™ in rat CNV

| Identification | 7 day mean | 7 days SEM | 14 day mean | 14 day SEM |
|---|---|---|---|---|
| A | 104.26 | 5.27 | 85.24 | 4.90 |
| B | 95.89 | 5.56 | 106.91 | 5.45 |
| C | 98.45 | 6.81 | 91.25 | 5.16 |
| D | 101.82 | 4.77 | 105.31 | 3.61 |
| E | 104.61 | 6.32 | 113.91 | 4.46 |

### Example 14

### DME Model—Prophetic Example

It is envisaged that antigen binding proteins disclosed herein will be effective in treating and/or preventing Diabetic Macular Edema (DME). This may be verified in a diabetic macula edema model in which DME and retinal vascular leak is observed following initiation of hyperglycemia as in Ishida, T. Usui and K. Yamashiro et al. (VEGF164 is proinflammatory in the diabetic retina, Invest Ophthalmol V is Sci 44 (2003), pp. 2155-2162).

Sequences

| | SEQ ID NO: | |
|---|---|---|
| Protein or polynucleotide description | DNA | Amino acid |
| anti-VEGF dAb DOM15-26-593 | | 1 |
| Anti-TNFα adnectin | | 2 |
| G4S Linker | | 3 |
| Linker | | 4 |
| Linker | | 5 |
| Linker | | 6 |
| Linker | | 7 |
| Linker | | 8 |
| Signal peptide sequence | | 9 |
| Anti-TNFα mAb (adalimumab) Heavy Chain | | 10 |
| Anti-TNFα mAb (adalimumab) Light Chain | 11 | 12 |
| Anti-TNFα mAb (adalimumab)-DOM15-26-593 Heavy | 13 | 14 |
| Chain | | |
| (DMS4000 mAbdAb heavy chain) | | |
| DOM 15-26-anti-TNFα mAb (adalimumab) Heavy Chain | — | 15 |
| Anti-TNFα mAb (adalimumab)-DOM15-10-11 Heavy | — | 16 |
| Chain | | |
| (DMS4031 mAbdAb heavy chain) | | |
| Anti-TNFR1 dAb (DOM1h-131-206) | | 17 |
| Anti-VEGFR2 adnectin | | 18 |
| Anti-VEGF anticalin | | 19 |
| Alternative Anti-VEGF antibody Heavy chain | | 20 |
| Anti-VEGF antibody (bevacizumab) Light chain | | 21 |
| Alternative Anti-VEGF antibody (bevacizumab) Heavy | | 22 |
| chain | | |
| anti-VEGF dAb DOM15-26 | | 23 |
| DOM15-26-593-Anti-TNFα mAb (adalimumab) Heavy | | 24 |
| Chain | | |
| Linker | | 25 |
| BPC1821 (CTLA4-Ig fused to anti-VEGFR2 adnectin via | 26 | 27 |
| a GS linker) | | |
| BPC1825 (CTLA4-Ig fused to an anti-VEGF dAb via a | 28 | 29 |
| GS linker) | | |
| Anti-TNFα mAb heavy chain | | 30 |
| Anti-TNFα mAb light chain | | 31 |
| Anti-TNFα mAb (Infliximab) Heavy chain | | 32 |
| Anti-TNFα mAb (Infliximab) Light chain | | 33 |
| TNFR-Fc fusion (Etanercept) | | 34 |
| Anti-TNFαVk dAb (PEP1-5-19) | | 35 |
| Anti-TNFα Vk dAb (PEP1-5-490) | | 36 |
| Anti-TNFα Vk dAb (PEP1-5-493) | | 37 |
| Anti-TNFα scFv (ESBA105) | | 38 |
| Anti-VEGF Fab (ranibizumab) Heavy Chain | | 39 |
| Anti-VEGF Fab (ranibizumab) Light Chain | | 40 |
| Anti-VEGF Vk dAb (DOM15-10-11) | | 44 |
| Anti-VEGF antibody (R84) Heavy chain | | 41 |
| Anti-VEGF antibody (R84) light chain | | 42 |
| VEGFR1/2 hybrid - Fc fusion (aflibercept - VEGF-Trap) | | 43 |
| CT01 | | 45 |
| Anti-TNFα mAb (adalimumab)-DOM15-26-593 Heavy | 46 | 47 |
| Chain FC disabled | | |
| (DMS4000 mAbdAb heavy chain Fc disabled) | | |
| EtanSTG593 | 48 | 49 |
| EtanTV4593 | 50 | 51 |
| AVG18 primer | 52 | |
| AVG19 primer | 53 | |
| AVG26 primer | 54 | |
| AVG21 primer | 55 | |
| AVG22 primer | 56 | |
| AVG36 primer | 57 | |
| AVG37 primer | 58 | |
| AVG25 primer | 59 | |
| AVG24 primer | 60 | |
| DOM15-26-593 - PEP1-5-19 in-line fusion | 61 | 62 |
| PEP1-5-19-DOM15-26-593 in-line fusion | 63 | 64 |
| DMS1571—a myc tagged Fc formatted version of the | | 65 |
| DOM 15-26-593 anti-VEGF dAb (exists as a dimer of this | | |
| sequence) | | |
| Linker | | 66 |
| Linker | | 67 |
| Linker | | 68 |
| Anti-TNFα mAb (adalimumab) Fc disabled-DOM15-26- | 141 | 69 |
| 593 Heavy Chain with GSTVAAPSGS linker | | |
| Anti-TNFα mAb (adalimumab) Fc disabled -DOM15-26- | 142 | 70 |
| 593 Heavy Chain with GS(TVAAPSGS) x2 linker | | |
| Anti-TNFα mAb (adalimumab) Fc disabled -DOM15-26- | 143 | 71 |
| 593 Heavy Chain with GS(TVAAPSGS) x3 linker | | |
| Anti-TNFα mAb (adalimumab) Fc disabled -DOM15-26- | 144 | 72 |
| 593 Heavy Chain with GS(TVAAPSGS) x4 linker | | |
| Etanercept-DOM15-26-593 | | 73 |
| Etanercept-DOM15-10-11 | | 74 |
| Etanercept-VEGF anticalin | | 75 |
| Infliximab-bevacizumab DVD-Ig heavy chain | | 76 |
| Infliximab-bevacizumab DVD-Ig light chain | | 77 |
| Infliximab-r84 DVD-Ig heavy chain | | 78 |
| Infliximab-r84 DVD-Ig light chain | | 79 |
| Infliximab-ranibizumab DVD-Fab | | 80 |
| Infliximab-ranibizumab DVD-Fab | | 81 |
| Infliximab-DOM15-26-593 mAb-dAb heavy chain | | 82 |
| Infliximab-DOM15-10-11 mAb-dAb heavy chain | | 83 |
| Infliximab-VEGF anticalin heavy chain | | 84 |
| Infliximab-DOM15-26-593 mAb-dAb light chain | | 85 |
| Infliximab-DOM15-10-11 mAb-dAb light chain | | 86 |
| Infliximab-VEGF anticalin light chain | | 87 |
| Adalimumab-bevacizumab DVD-Ig heavy chain | | 88 |
| Adalimumab-bevacizumab DVD-Ig light chain | | 89 |
| Adalimumab-r84 DVD-Ig heavy chain | | 90 |
| Adalimumab-r84 DVD-Ig light chain | | 91 |
| Adalimumab-ranibizumab DVD-Fab | | 92 |
| Adalimumab-ranibizumab DVD-Fab | | 93 |
| Adalimumab-VEGF anticalin heavy chain | | 94 |
| Adalimumab-DOM15-26-593 mAb-dAb light chain | | 95 |
| Adalimumab-DOM15-10-11 mAb-dAb light chain | | 96 |
| Adalimumab-VEGF anticalin light chain | | 97 |
| anti- TNFα mAb -bevacizumab DVD-Ig heavy chain | | 98 |
| anti- TNFα mAb -bevacizumab DVD-Ig light chain | | 99 |
| anti- TNFα mAb -r84 DVD Ig heavy chain | | 100 |
| anti- TNFα mAb -r84 DVD-Ig light chain | | 101 |
| anti- TNFα mAb -ranibizumab DVD-Fab heavy chain | | 102 |
| anti- TNFα mAb -ranibizumab DVD-Fab light chain | | 103 |
| anti- TNFα mAb -DOM15-26-593 mAb-dAb heavy chain | | 104 |
| anti- TNFα mAb -DOM15-10-11 mAb-dAb heavy chain | | 105 |
| anti- TNFα mAb -VEGF anticalin heavy chain | | 106 |
| anti- TNFα mAb -DOM15-26-593 mAb-dAb light chain | | 107 |
| anti- TNFα mAb -DOM15-10-11 mAb-dAb light chain | | 108 |
| anti- TNFα mAb -VEGF anticalin light chain | | 109 |
| ESBA105-bevacizumab DVD-Ig heavy chain | | 110 |
| ESBA105-bevacizumab DVD-Ig light chain | | 111 |
| ESBA105-r84 DVD-Ig heavy chain | | 112 |
| ESBA105-r84 DVD-Ig light chain | | 113 |
| ESBA105-ranibizumab DVD-Fab heavy chain | | 114 |
| ESBA105-ranibizumab DVD-Fab light chain | | 115 |
| ESBA105-DOM15-26-593 scFv-VH dAb | | 116 |
| ESBA105-DOM15-10-11 scFv-Vk dAb | | 117 |
| ESBA105-VEGF anticalin | | 118 |
| PEP1-5-19-DOM15-10-11 dAb-dAb | | 119 |
| PEP1-5-19-VEGF anticalin | | 120 |
| Anti-TNF adnectin-DOM15-26-593 | | 121 |
| Anti-TNF adnectin-DOM15-10-11 | | 122 |
| Anti-TNF adnectin-VEGF anticalin | | 123 |
| Bevacizumab-ESBA105 mAb-scFv, heavy chain | | 124 |
| Bevacizumab-ESBA105 mAb-scFv, light chain | | 125 |
| Bevacizumab-PEP1-5-19 mAb-dAb heavy chain | | 126 |
| Bevacizumab-PEP1-5-19 mAb-dAb light chain | | 127 |
| Bevacizumab-TNF adnectin heavy chain | | 128 |
| Bevacizumab-TNF adnectin light chain | | 129 |
| Aflibercept-ESBA105 | | 130 |
| Aflibercept-PEP1-5-19 | | 131 |
| Aflibercept-TNF adnectin | | 132 |
| DOM15-26-593-ESBA105 dAb-scFv | | 133 |
| DOM15-26-593-TNF adnectin | | 134 |
| DOM15-10-11-ESBA105 dAb-scFv | | 135 |
| DOM15-10-11-PEP1-5-19 dAb-dAb | | 136 |
| DOM15-10-11-TNF adnectin | | 137 |
| VEGF anticalin-ESBA105 | | 138 |
| VEGF anticalin-PEP1-5-19 | | 139 |
| VEGF anticalin-TNF adnectin | | 140 |
| Linker | | 145 |
| Linker | | 146 |
| Linker | | 147 |
| Linker | | 148 |
| Linker | | 149 |
| Linker | | 150 |
| Linker | | 151 |
| Linker | | 152 |
| Linker | | 153 |
| Linker | | 154 |
| Linker | | 155 |
| Linker | | 156 |
| Linker | | 157 |
| Linker | | 158 |
| Linker | | 159 |
| Linker | | 160 |
| Linker | | 161 |
| Linker | | 162 |
| BPC1801 (bispecific IGF1R-VEGFR2) heavy chain | | 163 |
| BPC1801 (bispecific IGF1R-VEGFR2) light chain | | 164 |
| BPC1824 (CTLA4-Ig-anti-IL-13 dAb fusion) | | 165 |

SEQ ID NO: 1
EVQLLVSGGGLVQPGGSLRLSCAASGFTFKAYPMMWVRQAPGKGLEWVS
EISPSGSYTYYAD
SVKGRFTISRDNSKNTLYLQMNSLRAEDTAVYYCAKDPRKLDYWGQGTL
VTVSS
SEQ ID NO: 2
VSDVPRDLEVVAATPTSLLISWDTHNAYNGYYRITYGETGGNSPVREFT
VPHPEVTATISGLKPGVDDTITVYAVTNHHMPLRIFGPISINHRT
SEQ ID NO: 3
GGGGS
SEQ ID NO: 4
TVAAPS
SEQ ID NO: 5
ASTKGPT
SEQ ID NO: 6
ASTKGPS
SEQ ID NO: 7
GS
SEQ ID NO: 8
TVAAPSGS
SEQ ID NO: 9
MGWSCIILFLVATATGVHS
SEQ ID NO: 10
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK
SEQ ID NO: 11
GATATCCAGATGACCCAGAGCCCCAGCAGCCTGAGCGCCTCTGTGGGCG
ATAGAGTGACCATCACCTGCCGGGCCAGCCAGGGCATCAGAAACTACCT
GGCCTGGTATCAGCAGAAGCCTGGCAAGGCCCCTAAGCTGCTGATCTAC
GCCGCCAGCACCCTGCAGAGCGGCGTGCCCAGCAGATTCAGCGGCAGCG
GCTCCGGCACCGACTTCACCCTGACCATCAGCAGCCTGCAGCCCGAGGA
CGTGGCCACCTACTACTGCCAGCGGTACAACAGAGCCCCTTACACCTTC
GGCCAGGGCACCAAGGTGGAGATCAAGCGTACGGTGGCCGCCCCCAGCG
TGTTCATCTTCCCCCCCAGCGATGAGCAGCTCAAGAGCGGCACCGCCAG
CGTGGTGTGTCTGCTGAACAACTTCTACCCCCGGGAGGCCAAAGTGCAG
TGGAAAGTGGACAACGCCCTGCAGAGCGGCAACAGCCAGGAGAGCGTGA
CCGAGCAGGACAGCAAGGACTCCACCTACAGCCTGAGCAGCACCCTGAC
CCTGAGCAAGGCCGACTACGAGAAGCACAAAGTGTACGCCTGCGAAGTG
ACCCACCAGGGCCTGTCCAGCCCCGTGACCAAGAGCTTCAACCGGGGCG
AGTGC
SEQ ID NO: 12
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 13
GAGGTGCAGCTGGTGGAGTCTGGCGGCGGACTGGTGCAGCCCGGCAGAA
GCCTGAGACTGAGCTGTGCCGCCAGCGGCTTCACCTTCGACGACTACGC
CATGCACTGGGTGAGGCAGGCCCCTGGCAAGGGCCTGGAGTGGGTGTCC
GCCATCACCTGGAATAGCGGCCACATCGACTACGCCGACAGCGTGGAGG
GCAGATTCACCATCAGCCGGGACAACGCCAAGAACAGCCTGTACCTGCA
GATGAACAGCCTGAGAGCCGAGGACACCGCCGTGTACTACTGTGCCAAG
GTGTCCTACCTGAGCACCGCCAGCAGCCTGGACTACTGGGGCCAGGGCA
CCCTGGTGACAGTCTCGAGCGCTAGCACCAAGGGCCCCAGCGTGTTCCC
CCTGGCCCCCAGCAGCAAGAGCACCAGCGGCGGCACAGCCGCCCTGGGC
TGCCTGGTGAAGGACTACTTCCCCGAGCCTGTGACCGTGTCCTGGAATA
GCGGAGCCCTGACCTCCGGCGTGCACACCTTCCCCGCCGTGCTGCAGAG
CAGCGGCCTGTACTCCCTGAGCAGCGTGGTGACCGTGCCCAGCAGCAGC
CTGGGCACCCAGACCTACATCTGCAACGTGAACCACAAGCCCAGCAACA
CCAAAGTGGACAAGAAAGTGGAGCCCAAGAGCTGCGATAAGACCCACAC
CTGCCCCCCCTGCCCTGCCCCCGAGCTGCTGGGCGGACCTAGCGTGTTC
CTGTTCCCCCCCAAGCCTAAGGACACCCTGATGATCAGCAGGACCCCCG
AAGTGACCTGCGTGGTGGTGGATGTGAGCCACGAGGACCCTGAAGTGAA
GTTCAACTGGTACGTGGACGGCGTGGAAGTGCACAACGCCAAGACCAAG
CCCAGAGAGGAGCAGTACAACAGCACCTACCGCGTGGTGTCTGTGCTGA
CCGTGCTGCACCAGGATTGGCTGAACGGCAAGGAGTACAAGTGCAAAGT
GAGCAACAAGGCCCTGCCTGCCCCTATCGAGAAAACCATCAGCAAGGCC
AAGGGCCAGCCTAGAGAGCCCCAGGTCTACACCCTGCCTCCCTCCAGAG
ATGAGCTGACCAAGAACCAGGTGTCCCTGACCTGTCTGGTGAAGGGCTT
CTACCCCAGCGACATCGCCGTGGAGTGGGAGAGCAACGGCCAGCCCGAG
AACAACTACAAGACCACCCCCCCTGTGCTGGACAGCGATGGCAGCTTCT
TCCTGTACTCCAAGCTGACCGTGGACAAGAGCAGATGGCAGCAGGGCAA
CGTGTTCAGCTGCAGCGTGATGCACGAGGCCCTGCACAATCACTACACC
CAGAAGAGTCTGAGCCTGTCCCCTGGCAAGTCGACCGGTGAGGTGCAGC
TGTTGGTGTCTGGGGGAGGCTTGGTACAGCCTGGGGGGTCCCTGCGTCT
CTCCTGTGCAGCCTCCGGATTCACCTTTAAGGCTTATCCGATGATGTGG
GTCCGCCAGGCTCCAGGGAAGGGTCTAGAGTGGGTTTCAGAGATTTCGC
CTTCGGGTTCTTATACATACTACGCAGACTCCGTGAAGGGCCGGTTCAC
CATCTCCCGCGACAATTCCAAGAACACGCTGTATCTGCAAATGAACAGC
CTGCGTGCCGAGGACACCGCGGTATATTACTGTGCGAAAGATCCTCGGA
AGTTAGACTACTGGGGTCAGGGAACCCTGGTCACCGTCTCGAGC
SEQ ID NO: 14
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGKSTGEVQLLVSGGGLVQPGGSLRLSCAASGFTFKAYPMMW
VRQAPGKGLEWVSEISPSGSYTYYADSVKGRFTISRDNSKNTLYLQMNS
LRAEDTAVYYCAKDPRKLDYWGQGTLVTVSS
SEQ ID NO: 15
EVQLLESGGGLVQPGGSLRLSCAASGFTFGAYPMMWVRQAPGKGLEWVS
EISPSGSYTYYADSVKGRFTISRDNSKNTLYLQMNSLRAEDTAVYYCAK
DPRKFDYWGQGTLVTVSSASTKGPSEVQLVESGGGLVQPGRSLRLSCAA
SGFTFDDYAMHWVRQAPGKGLEWVSAITWNSGHIDYADSVEGRFTISRD
NAKNSLYLQMNSLRAEDTAVYYCAKVSYLSTASSLDYWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
KSLTCLVGFYPSDIAVEWESNGQPENNYKTTPPVLDSDGSFFLYSKLTV
DKSRWQQGNVFSCSVMHEALHNHYTQKSLSLSPGK
SEQ ID NO: 16
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGKSTGDIQMTQSPSSLSASVGDRVTITCRASQWIGPELRWY
QQKPGKAPKLLIYHTSILQSGVPSRFSGSGSGTDFTLTISSLQPEDFAT
YYCQQYMFQPMTFGQGTKVEIKR
SEQ ID NO: 17
EVQLLESGGGLVQPGGSLRLSCAASGFTFAHETMVWVRQAPGKGLEWVS
HIPPDGQDPFYADSVKGRFTISRDNSKNTLYLQMNSLRAEDTAVYHCAL
LPKRGPWFDYWGQGTLVTVSS
SEQ ID NO: 18
EVVAATPTSLLISWRHPHFPTRYYRITYGETGGNSPVQEFTVPLQPPTA
TISGLKPGVDYTITVYAVTDGRNGRLLSIPISINYRT
SEQ ID NO: 19
DGGGIRRSMSGTWYLKAMTVDREFPEMNLESVTPMTLTLLKGHNLEAKV
TMLISGRCQEVKAVLGRTKERKKYTADGGKHVAYIIPSAVRDHVIFYSE
GQLHGKPVRGVKLVGRDPKNNLEALEDFEKAAGARGLSTESILIPRQSE
TCSPG
SEQ ID NO: 20
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDYWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
SEQ ID NO: 21
DIQMTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 22
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDVWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
SEQ ID NO: 23
EVQLLESGGGLVQPGGSLRLSCAASGFTFGAYPMMWVRQAPGKGLEWVS
EISPSGSYTYYADSVKGRFTISRDNSKNTLYLQMNSLRAEDTAVYYCAK
DPRKFDYWGQGTLVTVSS
SEQ ID NO: 24
EVQLLVSGGGLVQPGGSLRLSCAASGFTFKAYPMMWVRQAPGKGLEWVS
EISPSGSYTYYADSVKGRFTISRDNSKNTLYLQMNSLRAEDTAVYYCAK
DPRKLDYWGQGTLVTVSSASTKGPSEVQLVESGGGLVQPGRSLRLSCAA
SGFTFDDYAMHWVRQAPGKGLEWVSAITWNSGHIDYADSVEGRFTISRD
NAKNSLYLQMNSLRAEDTAVYYCAKVSYLSTASSLDYWGQGTLVTVSSA
STKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPGK
SEQ ID NO: 25
STG
SEQ ID NO: 26
ATGCATGTCGCCCAGCCAGCGGTGGTGCTGGCCAGCTCCCGCGGCATTG
CCTCCTTCGTGTGCGAGTACGCCAGCCCCGGCAAGGCCACCGAGGTGCG
CGTCACGGTGCTCCGCCAGGCCGATAGCCAGGTGACCGAAGTGTGTGCC
GCTACGTACATGATGGGGAACGAGCTGACCTTCCTGGACGACTCTATCT
GCACCGGGACCTCGAGCGGGAACCAGGTGAACCTGACCATCCAGGGCCT
GCGCGCGATGGACACGGGCCTGTACATCTGCAAGGTGGAGTTGATGTAC
CCCCCCCCGTACTACCTGGGGATCGGCAACGGCACGCAGATCTACGTCA
TCGACCCCGAACCTTGCCCTGACAGCGACCAGGAGCCCAAGTCTAGTGA
CAAGACCCATACCTCTCCCCCCAGCCCCGCTCCAGAGCTGCTGGGGGGC
TCCAGCGTGTTCCTGTTTCCCCCCAAGCCTAAGGACACCCTGATGATCT
CCAGAACCCCCGAGGTGACCTGCGTGGTCGTGGATGTGAGTCACGAGGA
CCCTGAGGTGAAGTTCAACTGGTACGTGGACGGGGTGGAGGTGCATAAC
GCCAAGACCAAGCCTCGCGAGGAGCAGTACAACAGTACCTACCGCGTGG
TGTCCGTGCTCACTGTGCTGCATCAGGACTGGCTGAACGGCAAGGAGTA
TAAGTGCAAGGTGTCTAACAAGGCCTTGCCCGCCCCCATCGAGAAAACA
ATCTCCAAGGCCAAAGGGCAGCCCAGGGAACCTCAGGTGTACACCCTCC
CTCCAAGCCGTGACGAGCTGACCAAGAACCAGGTCTCTCTGACCTGCTT
GGTGAAGGGCTTCTACCCTAGCGACATCGCTGTGGAGTGGGAGTCCAAC
GGGCAGCCCGAGAACAACTACAAAACCACCCCGCCCGTGCTGGACTCTG
ACGGCTCCTTCTTCCTGTACAGCAAACTGACCGTGGACAAGTCCAGGTG
GCAGCAGGGAAACGTGTTCAGCTGCAGCGTCATGCATGAGGCCCTGCAT
AACCATTACACACAGAAGAGCCTGTCCCTGAGCCCCGGCAAGGGATCCG
AGGTGGTGGCCGCCACCCCCACCAGCCTGCTGATTTCCTGGAGGCACCC
CCACTTCCCCACACGCTACTACAGGATCACCTACGGCGAGACCGGCGGC
AACAGCCCCGTGCAGGAGTTCACCGTGCCCCTGCAGCCTCCCACTGCCA
CCATCAGCGGCCTCAAGCCCGGCGTGGACTACACCATCACCGTGTACGC
CGTCACCGACGGAAGGAACGGCAGGCTGCTGAGCATCCCCATCAGCATC
AACTACAGGACC
SEQ ID NO: 27
MHVAQPAVVLASSRGIASFVCEYASPGKATEVRVTVLRQADSQVTEVCA
ATYMMGNELTFLDDSICTGTSSGNQVNLTIQGLRAMDTGLYICKVELMY
PPPYYLGIGNGTQIYVIDPEPCPDSDQEPKSSDKTHTSPPSPAPELLGG
SSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGKGSEVVAATPTSLLISWRHPHFPTRYYRITYGETGG
NSPVQEFTVPLQPPTATISGLKPGVDYTITVYAVTDGRNGRLLSIPISI
NYRT
SEQ ID NO: 28
ATGCATGTCGCCCAGCCAGCGGTGGTGCTGGCCAGCTCCCGCGGCATTG
CCTCCTTCGTGTGCGAGTACGCCAGCCCCGGCAAGGCCACCGAGGTGCG
CGTCACGGTGCTCCGCCAGGCCGATAGCCAGGTGACCGAAGTGTGTGCC
GCTACGTACATGATGGGGAACGAGCTGACCTTCCTGGACGACTCTATCT
GCACCGGGACCTCGAGCGGGAACCAGGTGAACCTGACCATCCAGGGCCT
GCGCGCGATGGACACGGGCCTGTACATCTGCAAGGTGGAGTTGATGTAC
CCCCCCCCGTACTACCTGGGGATCGGCAACGGCACGCAGATCTACGTCA
TCGACCCCGAACCTTGCCCTGACAGCGACCAGGAGCCCAAGTCTAGTGA
CAAGACCCATACCTCTCCCCCCAGCCCCGCTCCAGAGCTGCTGGGGGGC
TCCAGCGTGTTCCTGTTTCCCCCCAAGCCTAAGGACACCCTGATGATCT
CCAGAACCCCCGAGGTGACCTGCGTGGTCGTGGATGTGAGTCACGAGGA
CCCTGAGGTGAAGTTCAACTGGTACGTGGACGGGGTGGAGGTGCATAAC
GCCAAGACCAAGCCTCGCGAGGAGCAGTACAACAGTACCTACCGCGTGG
TGTCCGTGCTCACTGTGCTGCATCAGGACTGGCTGAACGGCAAGGAGTA
TAAGTGCAAGGTGTCTAACAAGGCCTTGCCCGCCCCCATCGAGAAAACA
ATCTCCAAGGCCAAAGGGCAGCCCAGGGAACCTCAGGTGTACACCCTCC
CTCCAAGCCGTGACGAGCTGACCAAGAACCAGGTCTCTCTGACCTGCTT
GGTGAAGGGCTTCTACCCTAGCGACATCGCTGTGGAGTGGGAGTCCAAC
GGGCAGCCCGAGAACAACTACAAAACCACCCCGCCCGTGCTGGACTCTG
ACGGCTCCTTCTTCCTGTACAGCAAACTGACCGTGGACAAGTCCAGGTG
GCAGCAGGGAAACGTGTTCAGCTGCAGCGTCATGCATGAGGCCCTGCAT
AACCATTACACACAGAAGAGCCTGTCCCTGAGCCCCGGCAAGGGATCCG
AGGTGCAGCTCCTGGTCAGCGGCGGCGGCCTGGTCCAGCCCGGAGGCTC
ACTGAGGCTGAGCTGCGCCGCTAGCGGCTTCACCTTCAAGGCCTACCCC
ATGATGTGGGTCAGGCAGGCCCCCGGCAAAGGCCTGGAGTGGGTGTCTG
AGATCAGCCCCAGCGGCAGCTACACCTACTACGCCGACAGCGTGAAGGG
CAGGTTCACCATCAGCAGGGACAACAGCAAGAACACCCTGTACCTGCAG
ATGAACTCTCTGAGGGCCGAGGACACCGCCGTGTACTACTGCGCCAAGG
ACCCCAGGAAGCTGGACTATTGGGGCCAGGGCACTCTGGTGACCGTGAG
CAGC
SEQ ID NO: 29
MHVAQPAVVLASSRGIASFVCEYASPGKATEVRVTVLRQADSQVTEVCA
ATYMMGNELTFLDDSICTGTSSGNQVNLTIQGLRAMDTGLYICKVELMY
PPPYYLGIGNGTQIYVIDPEPCPDSDQEPKSSDKTHTSPPSPAPELLGG
SSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGKGSEVQLLVSGGGLVQPGGSLRLSCAASGFTFKAYP
MMWVRQAPGKGLEWVSEISPSGSYTYYADSVKGRFTISRDNSKNTLYLQ
MNSLRAEDTAVYYCAKDPRKLDYWGQGTLVTVSS
SEQ ID NO: 30
QVQLVESGGGVVQPGRSLRLSCAASGFIFSSYAMHWVRQAPGNGLEWVA
FMSYDGSNKKYADSVKGRFTISRDNSKNTLYLQMNSLRAEDTAVYYCAR
DRGIAAGGNYYYYGMDVWGQGTTVTVSS
SEQ ID NO: 31
EIVLTQSPATLSLSPGERATLSCRASQSVYSYLAWYQQKPGQAPRLLIY
DASNRATGIPARFSGSGSGTDFTLTISSLEPEDFAVYYCQQRSNWPPFT
FGPGTKVDIKR
SEQ ID NO: 32
EVKLEESGGGLVQPGGSMKLSCVASGFIFSNHWMNWVRQSPEKGLEWVA
EIRSKSINSATHYAESVKGRFTISRDDSKSAVYLQMTDLRTEDTGVYYC
SRNYYGSTYDYWGQGTTLTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
SEQ ID NO: 33
DILLTQSPAILSVSPGERVSFSCRASQFVGSSIHWYQQRTNGSPRLLIK
YASESMSGIPSRFSGSGSGTDFTLSINTVESEDIADYYCQQSHSWPFTF
GSGTNLEVKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 34
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGDEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK
SEQ ID NO: 35
DIQMTQSPSSLSASVGDRVTITCRASQSIDSYLHWYQQKPGKAPKLLIY
SASELQSGVPSRFSGSGSGTDFTLTISSLQPEDFATYYCQQVVWRPFTF
GQGTKVEIKR
SEQ ID NO: 36
DIQMTQSPSSLSASVGDRVTITCTASQSIDSYLHWYQQKPGKAPKLLIY
SASNLETGVPSRFSGSGSGTDFTLTISSLQPEDFATYYCQQVVWRPFTF
GQGTKVEIKR
SEQ ID NO: 37
DIQMTQSPSSLSASVGDRVTITCRASQAIDSYLHWYQQKPGKAPKLLIY
SASNLETGVPSRFSGSGSGTDFTLTISSLLIPEDFATYYCQQVVWRPFT
FGQGTKVEIKR
SEQ ID NO: 38
DIVMTQSPSSLSASVGDRVTLTCTASQSVSNDVVWYQQRPGKAPKLLIY
SAFNRYTGVPSRFSGRGYGTDFTLTISSLQPEDVAVYYCQQDYNSPRTF
GQGTKLEVKRGGGGSGGGGSGGGGSSGGGSQVQLVQSGAEVKKPGASVK
VSCTASGYTFTHYGMNWVRQAPGKGLEWMGWINTYTGEPTYADKFKDRF
TFSLETSASTVYMELTSLTSDDTAVYYCARERGDAMDYWGQGTLVTVSS
SEQ ID NO: 39
EVQLVESGGGLVQPGGSLRLSCAASGYDFTHYGMNWVRQAPGKGLEWVG
WINTYTGEPTYAADFKRRFTFSLDTSKSTAYLQMNSLRAEDTAVYYCAK
YPYYYGTSHWYFDVWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHL
SEQ ID NO: 40
DIQLTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 41
QVQLVQSGAEVKKPGASVKVSCKASGGTFSSYAISWVRQAPGQGLEWMG
GFDPEDGETIYAQKFQGRVTMTEDTSTDTAYMELSSLRSEDTAVYYCAT
GRSMFRGVIIPFNGMDVWGQGTTVTVSS
SEQ ID NO: 42
DIRMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIKR
SEQ ID NO: 43
SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEKDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 44
DIQMTQSPSSLSASVGDRVTITCRASQWIGPELRWYQQKPGKAPKLLIY
HTSILQSGVPSRFSGSGSGTDFTLTISSLQPEDFATYYCQQYMFQPMTF
GQGTKVEIRG
SEQ ID NO: 45
EVVAATPTSLLISWRHPHFPTRYYRITYGETGGNSPVQEFTVPLQPPTA
TISGLKPGVDYTITVYAVTDGRNGRLLSIPISINYRT
SEQ ID NO: 46
ATGGGCTGGTCCTGCATCATCCTGTTTCTGGTGGCCACCGCCACCGGCG
TGCACAGCGAGGTGCAGCTGGTGGAGTCTGGCGGCGGACTGGTGCAGCC
CGGCAGAAGCCTGAGACTGAGCTGTGCCGCCAGCGGCTTCACCTTCGAC
GACTACGCCATGCACTGGGTGAGGCAGGCCCCTGGCAAGGGCCTGGAGT
GGGTGTCCGCCATCACCTGGAATAGCGGCCACATCGACTACGCCGACAG
CGTGGAGGGCAGATTCACCATCAGCCGGGACAACGCCAAGAACAGCCTG
TACCTGCAGATGAACAGCCTGAGAGCCGAGGACACCGCCGTGTACTACT
GTGCCAAGGTGTCCTACCTGAGCACCGCCAGCAGCCTGGACTACTGGGG
CCAGGGCACCCTGGTGACAGTCTCGAGCGCTAGCACCAAGGGCCCCAGC
GTGTTCCCCCTGGCCCCCAGCAGCAAGAGCACCAGCGGCGGCACAGCCG
CCCTGGGCTGCCTGGTGAAGGACTACTTCCCCGAGCCTGTGACCGTGTC
CTGGAATAGCGGAGCCCTGACCTCCGGCGTGCACACCTTCCCCGCCGTG
CTGCAGAGCAGCGGCCTGTACTCCCTGAGCAGCGTGGTGACCGTGCCCA
GCAGCAGCCTGGGCACCCAGACCTACATCTGCAACGTGAACCACAAGCC
CAGCAACACCAAAGTGGACAAGAAAGTGGAGCCCAAGAGCTGCGATAAG
ACCCACACCTGCCCCCCCTGCCCTGCCCCCGAGCTGGCCGGCGCCCCTA
GCGTGTTCCTGTTCCCCCCCAAGCCTAAGGACACCCTGATGATCAGCAG
GACCCCCGAAGTGACCTGCGTGGTGGTGGATGTGAGCCACGAGGACCCT
GAAGTGAAGTTCAACTGGTACGTGGACGGCGTGGAAGTGCACAACGCCA
AGACCAAGCCCAGAGAGGAGCAGTACAACAGCACCTACCGCGTGGTGTC
TGTGCTGACCGTGCTGCACCAGGATTGGCTGAACGGCAAGGAGTACAAG
TGCAAAGTGAGCAACAAGGCCCTGCCTGCCCCTATCGAGAAAACCATCA
GCAAGGCCAAGGGCCAGCCTAGAGAGCCCCAGGTCTACACCCTGCCTCC
CTCCAGAGATGAGCTGACCAAGAACCAGGTGTCCCTGACCTGTCTGGTG
AAGGGCTTCTACCCCAGCGACATCGCCGTGGAGTGGGAGAGCAACGGCC
AGCCCGAGAACAACTACAAGACCACCCCCCCTGTGCTGGACAGCGATGG
CAGCTTCTTCCTGTACTCCAAGCTGACCGTGGACAAGAGCAGATGGCAG
CAGGGCAACGTGTTCAGCTGCAGCGTGATGCACGAGGCCCTGCACAATC
ACTACACCCAGAAGAGTCTGAGCCTGTCCCCTGGCAAGTCGACCGGTGA
AGGTGCGCTGCTGGTGTCTGGCGGCGGACTGGTGCAGCCTGGCGGCAGC
CTGAGACTGAGCTGCGCCGCCAGCGGCTTCACCTTCAAGGCCTACCCCA
TGATGTGGGTGCGGCAGGCCCCTGGCAAGGGCCTGGAATGGGTGTCCGA
GATCAGCCCCAGCGGCAGCTACACCTACTACGCCGACAGCGTGAAGGGC
CGGTTCACCATCAGCCGGGACAACAGCAAGAACACCCTGTACCTGCAGA
TGAACAGCCTGCGGGCCGAGGACACCGCCGTGTACTACTGCGCCAAGGA
CCCCCGGAAGCTGGACTACTGGGGCCAGGGCACCCTGGTGACCGTGAGC
AGC
SEQ ID NO: 47
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELAGAPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGKSTGEVQLLVSGGGLVQPGGSLRLSCAASGFTFKAYPMMW
VRQAPGKGLEWVSEISPSGSYTYYADSVKGRFTISRDNSKNTLYLQMNS
LRAEDTAVYYCAKDPRKLDYWGQGTLVTVSS
SEQ ID NO: 48
CTGCCCGCTCAGGTGGCCTTCACTCCCTACGCCCCAGAGCCCGGCTCTA
CCTGCAGGCTGAGGGAGTACTACGACCAGACCGCCCAGATGTGCTGCAG
CAAGTGCAGCCCCGGCCAGCACGCCAAAGTGTTCTGCACCAAGACCAGC
GACACCGTGTGCGATAGCTGCGAGGACAGCACCTACACCCAGCTGTGGA
ACTGGGTCCCCGAGTGCCTGAGCTGCGGCTCTAGGTGTAGCAGCGACCA
GGTCGAGACCCAGGCCTGCACCAGGGAACAGAACCGGATCTGCACATGC
AGGCCCGGCTGGTACTGCGCCCTCAGCAAACAGGAGGGCTGCAGGCTGT
GTGCCCCCCTCAGGAAGTGCAGGCCCGGGTTTGGCGTGGCCAGGCCCGG
AACCGAGACTAGCGACGTGGTGTGCAAACCCTGCGCCCCCGGCACCTTC
AGCAATACCACTAGCAGCACCGACATCTGCAGGCCTCACCAGATCTGCA
ACGTGGTGGCCATTCCCGGCAACGCAAGCATGGACGCCGTGTGCACCAG
CACCAGCCCCACCAGGTCAATGGCCCCTGGAGCCGTGCATCTGCCCCAG
CCCGTGAGCACCAGAAGCCAGCACACCCAGCCTACCCCCGAGCCCAGCA
CCGCCCCTAGCACCAGCTTCCTGCTGCCTATGGGCCCCTCCCCTCCCGC
CGAGGGCTCAACCGGCGACGAACCCAAGAGCTGCGACAAGACCCACACC
TGCCCCCCCTGCCCCGCACCAGAACTCCTGGGCGGACCCAGCGTGTTCC
TGTTCCCCCCCAAGCCCAAGGACACCCTGATGATCAGCAGGACCCCCGA
GGTGACCTGTGTGGTGGTGGACGTGAGCCACGAGGACCCCGAGGTGAAG
TTCAACTGGTACGTGGACGGCGTGGAGGTGCACAACGCCAAGACCAAGC
CCAGGGAGGAGCAGTACAACAGCACCTACAGGGTGGTGAGCGTCCTGAC
CGTGCTGCACCAGGACTGGCTGAACGGCAAGGAGTACAAGTGCAAGGTG
AGCAACAAGGCCCTGCCCGCCCCCATCGAGAAGACCATCAGCAAGGCCA
AAGGCCAGCCCAGGGAGCCACAGGTGTACACACTGCCCCCCAGCAGGGA
GGAGATGACCAAGAACCAGGTGAGCCTGACCTGCCTGGTGAAGGGCTTC
TATCCCAGCGATATCGCCGTGGAGTGGGAGAGCAACGGCCAGCCCGAGA
ACAACTACAAGACCACCCCCCCCGTCCTGGACTCCGACGGGAGCTTCTT
CCTGTACAGCAAGCTGACCGTGGACAAGAGCAGGTGGCAGCAGGGCAAC
GTGTTCAGCTGCAGCGTGATGCACGAGGCCCTGCACAACCACTACACCC
AGAAGTCCCTGAGCCTGAGCCCCGGCAAGTCGACCGGTGAGGTGCAGCT
GCTGGTGTCTGGCGGCGGACTGGTGCAGCCTGGCGGCAGCCTGAGACTG
AGCTGCGCCGCCAGCGGCTTCACCTTCAAGGCCTACCCCATGATGTGGG
TGCGGCAGGCCCCTGGCAAGGGCCTGGAATGGGTGTCCGAGATCAGCCC
CAGCGGCAGCTACACCTACTACGCCGACAGCGTGAAGGGCCGGTTCACC
ATCAGCCGGGACAACAGCAAGAACACCCTGTACCTGCAGATGAACAGCC
TGCGGGCCGAGGACACCGCCGTGTACTACTGCGCCAAGGACCCCCGGAA
GCTGGACTACTGGGGCCAGGGCACCCTGGTGACCGTGAGCAGC
SEQ ID NO: 49
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGDEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGKSTGEVQLLVSGGGLVQPGGSLRL
SCAASGFTFKAYPMMWVRQAPGKGLEWVSEISPSGSYTYYADSVKGRFT
ISRDNSKNTLYLQMNSLRAEDTAVYYCAKDPRKLDYWGQGTLVTVSS
SEQ ID NO: 50
CTGCCCGCTCAGGTGGCCTTCACTCCCTACGCCCCAGAGCCCGGCTCTA
CCTGCAGGCTGAGGGAGTACTACGACCAGACCGCCCAGATGTGCTGCAG
CAAGTGCAGCCCCGGCCAGCACGCCAAAGTGTTCTGCACCAAGACCAGC
GACACCGTGTGCGATAGCTGCGAGGACAGCACCTACACCCAGCTGTGGA
ACTGGGTCCCCGAGTGCCTGAGCTGCGGCTCTAGGTGTAGCAGCGACCA
GGTCGAGACCCAGGCCTGCACCAGGGAACAGAACCGGATCTGCACATGC
AGGCCCGGCTGGTACTGCGCCCTCAGCAAACAGGAGGGCTGCAGGCTGT
GTGCCCCCCTCAGGAAGTGCAGGCCCGGGTTTGGCGTGGCCAGGCCCGG
AACCGAGACTAGCGACGTGGTGTGCAAACCCTGCGCCCCCGGCACCTTC
AGCAATACCACTAGCAGCACCGACATCTGCAGGCCTCACCAGATCTGCA
ACGTGGTGGCCATTCCCGGCAACGCAAGCATGGACGCCGTGTGCACCAG
CACCAGCCCCACCAGGTCAATGGCCCCTGGAGCCGTGCATCTGCCCCAG
CCCGTGAGCACCAGAAGCCAGCACACCCAGCCTACCCCCGAGCCCAGCA
CCGCCCCTAGCACCAGCTTCCTGCTGCCTATGGGCCCCTCCCCTCCCGC
CGAGGGCTCAACCGGCGACGAACCCAAGAGCTGCGACAAGACCCACACC
TGCCCCCCCTGCCCCGCACCAGAACTCCTGGGCGGACCCAGCGTGTTCC
TGTTCCCCCCCAAGCCCAAGGACACCCTGATGATCAGCAGGACCCCCGA
GGTGACCTGTGTGGTGGTGGACGTGAGCCACGAGGACCCCGAGGTGAAG
TTCAACTGGTACGTGGACGGCGTGGAGGTGCACAACGCCAAGACCAAGC
CCAGGGAGGAGCAGTACAACAGCACCTACAGGGTGGTGAGCGTCCTGAC
CGTGCTGCACCAGGACTGGCTGAACGGCAAGGAGTACAAGTGCAAGGTG
AGCAACAAGGCCCTGCCCGCCCCCATCGAGAAGACCATCAGCAAGGCCA
AAGGCCAGCCCAGGGAGCCACAGGTGTACACACTGCCCCCCAGCAGGGA
GGAGATGACCAAGAACCAGGTGAGCCTGACCTGCCTGGTGAAGGGCTTC
TATCCCAGCGATATCGCCGTGGAGTGGGAGAGCAACGGCCAGCCCGAGA
ACAACTACAAGACCACCCCCCCCGTCCTGGACTCCGACGGGAGCTTCTT
CCTGTACAGCAAGCTGACCGTGGACAAGAGCAGGTGGCAGCAGGGCAAC
GTGTTCAGCTGCAGCGTGATGCACGAGGCCCTGCACAACCACTACACCC
AGAAGTCCCTGAGCCTGAGCCCCGGCAAGACCGTGGCGGCGCCCAGCAC
GGTGGCCGCCCCCTCCACCGTCGCCGCGCCAAGCACCGTGGCTGCTCCG
CTCGACCGGTGAGGTGCAGTGCTGGTGTCTGGCGGCGGACTGGTGCAGC
CTGGCGGCAGCCTGAGACTGAGCTGCGCCGCCAGCGGCTTCACCTTCAA
GGCCTACCCCATGATGTGGGTGCGGCAGGCCCCTGGCAAGGGCCTGGAA
TGGGTGTCCGAGATCAGCCCCAGCGGCAGCTACACCTACTACGCCGACA
GCGTGAAGGGCCGGTTCACCATCAGCCGGGACAACAGCAAGAACACCCT
GTACCTGCAGATGAACAGCCTGCGGGCCGAGGACACCGCCGTGTACTAC
TGCGCCAAGGACCCCCGGAAGCTGGACTACTGGGGCCAGGGCACCCTGG
TGACCGTGAGCAGC
SEQ ID NO: 51
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGDEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGKTVAAPSTVAAPSTVAAPSTVAAP
STGEVQLLVSGGGLVQPGGSLRLSCAASGFTFKAYPMMWVRQAPGKGLE
WVSEISPSGSYTYYADSVKGRFTISRDNSKNTLYLQMNSLRAEDTAVYY
CAKDPRKLDYWGQGTLVTVSS
SEQ ID NO: 52
ATTATGGGATCCACCGGCGAGGTGCAGCTGTTGGTGT
SEQ ID NO: 53
GCTGGGGCCCTTGGTGCTAGCGCTCGAGACGGTGACCAGG
SEQ ID NO: 54
CTCGAGCGCTAGCACCAAGGGCCCCAGCGACATCCAGATGACCC
SEQ ID NO: 55
TTATGTCAAGCTTTTACCGTTTGATTTCCACCTTGGT
SEQ ID NO: 56
ATTATGGGATCCACCGGCGACATCCAGATGACCCAGTCTCC
SEQ ID NO: 57
GCGCCGCCACCGTACGTTTGATTTCCACCTTGGTCCC
SEQ ID NO: 58
CAAACGTACGGTGGCGGCGCCGAGCGAGGTGCAGCTGTTGGTGTC
SEQ ID NO: 59
TTATGTCAAGCTTTTAGCTCGAGACGGTGACCAG
SEQ ID NO: 60
GGTGGAAATCAAACGTACGGTGGCGGCGCCGAGCGA
SEQ ID NO: 61
GAGGTGCAGCTGTTGGTGTCTGGGGGAGGCTTGGTACAGCCTGGGGGGT
CCCTGCGTCTCTCCTGTGCAGCCTCCGGATTCACCTTTAAGGCTTATCC
GATGATGTGGGTCCGCCAGGCTCCAGGGAAGGGTCTAGAGTGGGTTTCA
GAGATTTCGCCTTCGGGTTCTTATACATACTACGCAGACTCCGTGAAGG
GCCGGTTCACCATCTCCCGCGACAATTCCAAGAACACGCTGTATCTGCA
AATGAACAGCCTGCGTGCCGAGGACACCGCGGTATATTACTGTGCGAAA
GATCCTCGGAAGTTAGACTACTGGGGTCAGGGAACCCTGGTCACCGTCT
CGAGCGCTAGCACCAAGGGCCCCAGCGACATCCAGATGACCCAGTCTCC
ATCCTCTCTGTCTGCATCTGTAGGAGACCGTGTCACCATCACTTGCCGG
GCAAGTCAGAGCATTGATAGTTATTTACATTGGTACCAGCAGAAACCAG
GGAAAGCCCCTAAGCTCCTGATCTATAGTGCATCCGAGTTGCAAAGTGG
GGTCCCATCACGTTTCAGTGGCAGTGGATCTGGGACAGATTTCACTCTC
ACCATCAGCAGTCTGCAACCTGAAGATTTTGCTACGTACTACTGTCAAC
AGGTTGTGTGGCGTCCTTTTACGTTCGGCCAAGGGACCAAGGTGGAAAT
CAAACGG
SEQ ID NO: 62
EVQLLVSGGGLVQPGGSLRLSCAASGFTFKAYPMMWVRQAPGKGLEWVS
EISPSGSYTYYADSVKGRFTISRDNSKNTLYLQMNSLRAEDTAVYYCAK
DPRKLDYWGQGTLVTVSSASTKGPSDIQMTQSPSSLSASVGDRVTITCR
ASQSIDSYLHWYQQKPGKAPKLLIYSASELQSGVPSRFSGSGSGTDFTL
TISSLQPEDFATYYCQQVVWRPFTFGQGTKVEIKR
SEQ ID NO: 63
GACATCCAGATGACCCAGTCTCCATCCTCTCTGTCTGCATCTGTAGGAG
ACCGTGTCACCATCACTTGCCGGGCAAGTCAGAGCATTGATAGTTATTT
ACATTGGTACCAGCAGAAACCAGGGAAAGCCCCTAAGCTCCTGATCTAT
AGTGCATCCGAGTTGCAAAGTGGGGTCCCATCACGTTTCAGTGGCAGTG
GATCTGGGACAGATTTCACTCTCACCATCAGCAGTCTGCAACCTGAAGA
TTTTGCTACGTACTACTGTCAACAGGTTGTGTGGCGTCCTTTTACGTTC
GGCCAAGGGACCAAGGTGGAAATCAAACGTACGGTGGCGGCGCCGAGCG
AGGTGCAGCTGTTGGTGTCTGGGGGAGGCTTGGTACAGCCTGGGGGGTC
CCTGCGTCTCTCCTGTGCAGCCTCCGGATTCACCTTTAAGGCTTATCCG
ATGATGTGGGTCCGCCAGGCTCCAGGGAAGGGTCTAGAGTGGGTTTCAG
AGATTTCGCCTTCGGGTTCTTATACATACTACGCAGACTCCGTGAAGGG
CCGGTTCACCATCTCCCGCGACAATTCCAAGAACACGCTGTATCTGCAA
ATGAACAGCCTGCGTGCCGAGGACACCGCGGTATATTACTGTGCGAAAG
ATCCTCGGAAGTTAGACTACTGGGGTCAGGGAACCCTGGTCACCGTCTC
GAGC
SEQ ID NO: 64
DIQMTQSPSSLSASVGDRVTITCRASQSIDSYLHWYQQKPGKAPKLLIY
SASELQSGVPSRFSGSGSGTDFTLTISSLQPEDFATYYCQQVVWRPFTF
LGQGTKVEIKRTVAAPSEVQLLVSGGGVQPGGSLRLSCAASGFTFKAYP
MMWVRQAPGKGLEWVSEISPSGSYTYYADSVKGRFTISRDNSKNTLYLQ
MNSLRAEDTAVYYCAKDPRKLDYWGQGTLVTVSS
SEQ ID NO: 65
EVQLLVSGGGLVQPGGSLRLSCAASGFTFKAYPMMWVRQAPGKGLEWVS
EISPSGSYTYYADSVKGRFTISRDNSKNTLYLQMNSLRAEDTAVYYCAK
DPRKLDYWGQGTLVTVSSASTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
GSEQKLISEEDLN
SEQ ID NO: 66
GSTVAAPSGSTVAAPSGS
SEQ ID NO: 67
GSTVAAPSGSTVAAPSGSTVAAPSGS
SEQ ID NO: 68
GSTVAAPSGSTVAAPSGSTVAAPSGSTVAAPSGS
SEQ ID NO: 69
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELAGAPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGKGSTVAAPSGSEVQLLVSGGGLVQPGGSLRLSCAASGFTF
KAYPMMWVRQAPGKGLEWVSEISPSGSYTYYADSVKGRFTISRDNSKNT
LYLQMNSLRAEDTAVYYCAKDPRKLDYWGQGTLVTVSS
SEQ ID NO: 70
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELAGAPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGKGSTVAAPSGSTVAAPSGSEVQLLVSGGGLVQPGGSLRLS
CAASGFTFKAYPMMWVRQAPGKGLEWVSEISPSGSYTYYADSVKGRFTI
SRDNSKNTLYLQMNSLRAEDTAVYYCAKDPRKLDYWGQGTLVTVSS
SEQ ID NO: 71
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELAGAPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGKGSTVAAPSGSTVAAPSGSTVAAPSGSEVQLLVSGGGLVQ
PGGSLRLSCAASGFTFKAYPMMWVRQAPGKGLEWVSEISPSGSYTYYAD
SVKGRFTISRDNSKNTLYLQMNSLRAEDTAVYYCAKDPRKLDYWGQGTL
VTVSS
SEQ ID NO: 72
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELAGAPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGKGSTVAAPSGSTVAAPSGSTVAAPSGSTVAAPSGSEVQLL
VSGGGLVQPGGSLRLSCAASGFTFKAYPMMWVRQAPGKGLEWVSEISPS
GSYTYYADSVKGRFTISRDNSKNTLYLQMNSLRAEDTAVYYCAKDPRKL
DYWGQGTLVTVSS
SEQ ID NO: 73
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGDEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGKTVAAPSEVQLLVSGGGLVQPGGS
LRLSCAASGFTFKAYPMMWVRQAPGKGLEWVSEISPSGSYTYYADSVKG
RFTISRDNSKNTLYLQMNSLRAEDTAVYYCAKDPRKLDYWGQGTLVTVS
S
SEQ ID NO: 74
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGDEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGKTVAAPSDIQMTQSPSSLSASVGD
RVTITCRASQWIGPELRWYQQKPGKAPKLLIYHTSILQSGVPSRFSGSG
SGTDFTLTISSLQPEDFATYYCQQYMFQPMTFGQGTKVEIKR
SEQ ID NO: 75
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGDEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGKTVAAPSDGGGIRRSMSGTWYLKA
MTVDREFPEMNLESVTPMTLTLLKGHNLEAKVTMLISGRCQEVKAVLGR
TKERKKYTADGGKHVAYIIPSAVRDHVIFYSEGQLHGKPVRGVKLVGRD
PKNNLEALEDFEKAAGARGLSTESILIPRQSETCSPG
SEQ ID NO: 76
EVKLEESGGGLVQPGGSMKLSCVASGFIFSNHWMNWVRQSPEKGLEWVA
EIRSKSINSATHYAESVKGRFTISRDDSKSAVYLQMTDLRTEDTGVYYC
SRNYYGSTYDYWGQGTTLTVSSASTKGPSEVQLVESGGGLVQPGGSLRL
SCAASGYTFTNYGMNWVRQAPGKGLEWVGWINTYTGEPTYAADFKRRFT
FSLDTSKSTAYLQMNSLRAEDTAVYYCAKYPHYYGSSHWYFDVWGQGTL
VTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFL
YSKLTVDKSRWQQGNVFSCSV
SEQ ID NO: 77
DILLTQSPAILSVSPGERVSFSCRASQFVGSSIHWYQQRTNGSPRLLIK
YASESMSGIPSRFSGSGSGTDFTLSINTVESEDIADYYCQQSHSWPFTF
GSGTNLEVKRTVAAPSDIQMTQSPSSLSASVGDRVTITCSASQDISNYL
NWYQQKPGKAPKVLIYFTSSLHSGVPSRFSGSGSGTDFTLTISSLQPED
FATYYCQQYSTVPWTFGQGTKVEIKRTVAAPSVFIFPPSDEQLKSGTAS
VVCLLNNFYPREAKVQWKVDNALQSGNSQESVTEQDSKDSTYSLSSTLT
LSKADYEKHKVYACEVTHQGLSSPVTKSFNRGEC
SEQ ID NO: 78
EVKLEESGGGLVQPGGSMKLSCVASGFIFSNHWMNWVRQSPEKGLEWVA
EIRSKSINSATHYAESVKGRFTISRDDSKSAVYLQMTDLRTEDTGVYYC
SRNYYGSTYDYWGQGTTLTVSSASTKGPSQVQLVQSGAEVKKPGASVKV
SCKASGGTFSSYAISWVRQAPGQGLEWMGGFDPEDGETIYAQKFQGRVT
MTEDTSTDTAYMELSSLRSEDTAVYYCATGRSMFRGVIIPFNGMDVWGQ
GTTVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO: 79
DILLTQSPAILSVSPGERVSFSCRASQFVGSSIHWYQQRTNGSPRLLIK
YASESMSGIPSRFSGSGSGTDFTLSINTVESEDIADYYCQQSHSWPFTF
GSGTNLEVKRTVAAPSDIRMTQSPSSLSASVGDRVTITCRASQSISSYL
NWYQQKPGKAPKLLIYAASSLQSGVPSRFSGSGSGTDFTLTISSLQPED
FATYYCQQSYSTPLTFGGGTKVEIKRTVAAPSVFIFPPSDEQLKSGTAS
VVCLLNNFYPREAKVQWKVDNALQSGNSQESVTEQDSKDSTYSLSSTLT
LSKADYEKHKVYACEVTHQGLSSPVTKSFNRGEC
SEQ ID NO: 80
EVKLEESGGGLVQPGGSMKLSCVASGFIFSNHWMNWVRQSPEKGLEWVA
EIRSKSINSATHYAESVKGRFTISRDDSKSAVYLQMTDLRTEDTGVYYC
SRNYYGSTYDYWGQGTTLTVSSASTKGPSEVQLVESGGGLVQPGGSLRL
SCAASGYDFTHYGMNWVRQAPGKGLEWVGWINTYTGEPTYAADFKRRFT
FSLDTSKSTAYLQMNSLRAEDTAVYYCAKYPYYYGTSHWYFDVWGQGTL
VTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTK
VDKKVEPKSCDKTHL
SEQ ID NO: 81
DILLTQSPAILSVSPGERVSFSCRASQFVGSSIHWYQQRTNGSPRLLIK
YASESMSGIPSRFSGSGSGTDFTLSINTVESEDIADYYCQQSHSWPFTF
GSGTNLEVKRTVAAPSDIQLTQSPSSLSASVGDRVTITCSASQDISNYL
NWYQQKPGKAPKVLIYFTSSLHSGVPSRFSGSGSGTDFTLTISSLQPED
FATYYCQQYSTVPWTFGQGTKVEIKRTVAAPSVFIFPPSDEQLKSGTAS
VVCLLNNFYPREAKVQWKVDNALQSGNSQESVTEQDSKDSTYSLSSTLT
LSKADYEKHKVYACEVTHQGLSSPVTKSFNRGEC
SEQ ID NO: 82
EVKLEESGGGLVQPGGSMKLSCVASGFIFSNHWMNWVRQSPEKGLEWVA
EIRSKSINSATHYAESVKGRFTISRDDSKSAVYLQMTDLRTEDTGVYYC
SRNYYGSTYDYWGQGTTLTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGKTVAAPSEVQLLVSGGGLVQPGGSLRLSCAASGFTFKAYPM
MWVRQAPGKGLEWVSEISPSGSYTYYADSVKGRFTISRDNSKNTLYLQM
NSLRAEDTAVYYCAKDPRKLDYWGQGTLVTVSS
SEQ ID NO: 83
EVKLEESGGGLVQPGGSMKLSCVASGFIFSNHWMNWVRQSPEKGLEWVA
EIRSKSINSATHYAESVKGRFTISRDDSKSAVYLQMTDLRTEDTGVYYC
SRNYYGSTYDYWGQGTTLTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGKTVAAPSDIQMTQSPSSLSASVGDRVTITCRASQWIGPELR
WYQQKPGKAPKLLIYHTSILQSGVPSRFSGSGSGTDFTLTISSLQPEDF
ATYYCQQYMFQPMTFGQGTKVEIKR
SEQ ID NO: 84
EVKLEESGGGLVQPGGSMKLSCVASGFIFSNHWMNWVRQSPEKGLEWVA
EIRSKSINSATHYAESVKGRFTISRDDSKSAVYLQMTDLRTEDTGVYYC
SRNYYGSTYDYWGQGTTLTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGKTVAAPSDGGGIRRSMSGTWYLKAMTVDREFPEMNLESVTP
MTLTLLKGHNLEAKVTMLISGRCQEVKAVLGRTKERKKYTADGGKHVAY
IIPSAVRDHVIFYSEGQLHGKPVRGVKLVGRDPKNNLEALEDFEKAAGA
RGLSTESILIPRQSETCSPG
SEQ ID NO: 85
DILLTQSPAILSVSPGERVSFSCRASQFVGSSIHWYQQRTNGSPRLLIK
YASESMSGIPSRFSGSGSGTDFTLSINTVESEDIADYYCQQSHSWPFTF
GSGTNLEVKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSEVQLLVSGGGLVQPGGSLRLSCAAS
GFTFKAYPMMWVRQAPGKGLEWVSEISPSGSYTYYADSVKGRFTISRDN
SKNTLYLQMNSLRAEDTAVYYCAKDPRKLDYWGQGTLVTVSS
SEQ ID NO: 86
DILLTQSPAILSVSPGERVSFSCRASQFVGSSIHWYQQRTNGSPRLLIK
YASESMSGIPSRFSGSGSGTDFTLSINTVESEDIADYYCQQSHSWPFTF
GSGTNLEVKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSDIQMTQSPSSLSASVGDRVTITCRA
SQWIGPELRWYQQKPGKAPKLLIYHTSILQSGVPSRFSGSGSGTDFTLT
ISSLQPEDFATYYCQQYMFQPMTFGQGTKVEIKR
SEQ ID NO: 87
DILLTQSPAILSVSPGERVSFSCRASQFVGSSIHWYQQRTNGSPRLLIK
YASESMSGIPSRFSGSGSGTDFTLSINTVESEDIADYYCQQSHSWPFTF
GSGTNLEVKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSDGGGIRRSMSGTWYLKAMTVDREFP
EMNLESVTPMTLTLLKGHNLEAKVTMLISGRCQEVKAVLGRTKERKKYT
ADGGKHVAYIIPSAVRDHVIFYSEGQLHGKPVRGVKLVGRDPKNNLEAL
EDFEKAAGARGLSTESILIPRQSETCSPG
SEQ ID NO: 88
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSEVQLVESGGGLVQPGGSLR
LSCAASGYTFTNYGMNWVRQAPGKGLEWVGWINTYTGEPTYAADFKRRF
TFSLDTSKSTAYLQMNSLRAEDTAVYYCAKYPHYYGSSHWYFDVWGQGT
LVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 89
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSDIQMTQSPSSLSASVGDRVTITCSASQDISNYL
NWYQQKPGKAPKVLIYFTSSLHSGVPSRFSGSGSGTDFTLTISSLQPED
FATYYCQQYSTVPWTFGQGTKVEIKRTVAAPSVFIFPPSDEQLKSGTAS
VVCLLNNFYPREAKVQWKVDNALQSGNSQESVTEQDSKDSTYSLSSTLT
LSKADYEKHKVYACEVTHQGLSSPVTKSFNRGEC
SEQ ID NO: 90
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSQVQLVQSGAEVKKPGASVK
VSCKASGGTFSSYAISWVRQAPGQGLEWMGGFDPEDGETIYAQKFQGRV
TMTEDTSTDTAYMELSSLRSEDTAVYYCATGRSMFRGVIIPFNGMDVWG
QGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO: 91
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSDIRMTQSPSSLSASVGDRVTITCRASQSISSYL
NWYQQKPGKAPKLLIYAASSLQSGVPSRFSGSGSGTDFTLTISSLQPED
FATYYCQQSYSTPLTFGGGTKVEIKRTVAAPSVFIFPPSDEQLKSGTAS
VVCLLNNFYPREAKVQWKVDNALQSGNSQESVTEQDSKDSTYSLSSTLT
LSKADYEKHKVYACEVTHQGLSSPVTKSFNRGEC
SEQ ID NO: 92
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSEVQLVESGGGLVQPGGSLR
LSCAASGYDFTHYGMNWVRQAPGKGLEWVGWINTYTGEPTYAADFKRRF
TFSLDTSKSTAYLQMNSLRAEDTAVYYCAKYPYYYGTSHWYFDVWGQGT
LVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNT
KVDKKVEPKSCDKTHL
SEQ ID NO: 93
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSDIQLTQSPSSLSASVGDRVTITCSASQDISNYL
NWYQQKPGKAPKVLIYFTSSLHSGVPSRFSGSGSGTDFTLTISSLQPED
FATYYCQQYSTVPWTFGQGTKVEIKRTVAAPSVFIFPPSDEQLKSGTAS
VVCLLNNFYPREAKVQWKVDNALQSGNSQESVTEQDSKDSTYSLSSTLT
LSKADYEKHKVYACEVTHQGLSSPVTKSFNRGEC
SEQ ID NO: 94
EVQLVESGGGLVQPGRSLRLSCAASGFTFDDYAMHWVRQAPGKGLEWVS
AITWNSGHIDYADSVEGRFTISRDNAKNSLYLQMNSLRAEDTAVYYCAK
VSYLSTASSLDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGKTVAAPSDGGGIRRSMSGTWYLKAMTVDREFPEMNLESVT
PMTLTLLKGHNLEAKVTMLISGRCQEVKAVLGRTKERKKYTADGGKHVA
YIIPSAVRDHVIFYSEGQLHGKPVRGVKLVGRDPKNNLEALEDFEKAAG
ARGLSTESILIPRQSETCSPG
SEQ ID NO: 95
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSEVQLLVSGGGLVQPGGSLRLSCAAS
GFTFKAYPMMWVRQAPGKGLEWVSEISPSGSYTYYADSVKGRFTISRDN
SKNTLYLQMNSLRAEDTAVYYCAKDPRKLDYWGQGTLVTVSS
SEQ ID NO: 96
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSDIQMTQSPSSLSASVGDRVTITCRA
SQWIGPELRWYQQKPGKAPKLLIYHTSILQSGVPSRFSGSGSGTDFTLT
ISSLQPEDFATYYCQQYMFQPMTFGQGTKVEIKR
SEQ ID NO: 97
DIQMTQSPSSLSASVGDRVTITCRASQGIRNYLAWYQQKPGKAPKLLIY
AASTLQSGVPSRFSGSGSGTDFTLTISSLQPEDVATYYCQRYNRAPYTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSDGGGIRRSMSGTWYLKAMTVDREFP
EMNLESVTPMTLTLLKGHNLEAKVTMLISGRCQEVKAVLGRTKERKKYT
ADGGKHVAYIIPSAVRDHVIFYSEGQLHGKPVRGVKLVGRDPKNNLEAL
EDFEKAAGARGLSTESILIPRQSETCSPG
SEQ ID NO: 98
QVQLVESGGGVVQPGRSLRLSCAASGFIFSSYAMHWVRQAPGNGLEWVA
FMSYDGSNKKYADSVKGRFTISRDNSKNTLYLQMNSLRAEDTAVYYCAR
DRGIAAGGNYYYYGMDVWGQGTTVTVSSASTKGPSEVQLVESGGGLVQP
GGSLRLSCAASGYTFTNYGMNWVRQAPGKGLEWVGWINTYTGEPTYAAD
FKRRFTFSLDTSKSTAYLQMNSLRAEDTAVYYCAKYPHYYGSSHWYFDV
WGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNH
KPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 99
EIVLTQSPATLSLSPGERATLSCRASQSVYSYLAWYQQKPGQAPRLLIY
DASNRATGIPARFSGSGSGTDFTLTISSLEPEDFAVYYCQQRSNWPPFT
FGPGTKVDIKRTVAAPSDIQMTQSPSSLSASVGDRVTITCSASQDISNY
LNWYQQKPGKAPKVLIYFTSSLHSGVPSRFSGSGSGTDFTLTISSLQPE
DFATYYCQQYSTVPWTFGQGTKVEIKRTVAAPSVFIFPPSDEQLKSGTA
SVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 100
QVQLVESGGGVVQPGRSLRLSCAASGFIFSSYAMHWVRQAPGNGLEWVA
FMSYDGSNKKYADSVKGRFTISRDNSKNTLYLQMNSLRAEDTAVYYCAR
DRGIAAGGNYYYYGMDVWGQGTTVTVSSASTKGPSQVQLVQSGAEVKKP
GASVKVSCKASGGTFSSYAISWVRQAPGQGLEWMGGFDPEDGETIYAQK
FQGRVTMTEDTSTDTAYMELSSLRSEDTAVYYCATGRSMFRGVIIPFNG
MDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K
SEQ ID NO: 101
EIVLTQSPATLSLSPGERATLSCRASQSVYSYLAWYQQKPGQAPRLLIY
DASNRATGIPARFSGSGSGTDFTLTISSLEPEDFAVYYCQQRSNWPPFT
FGPGTKVDIKRTVAAPSDIRMTQSPSSLSASVGDRVTITCRASQSISSY
LNWYQQKPGKAPKLLIYAASSLQSGVPSRFSGSGSGTDFTLTISSLQPE
DFATYYCQQSYSTPLTFGGGTKVEIKRTVAAPSVFIFPPSDEQLKSGTA
SVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 102
QVQLVESGGGVVQPGRSLRLSCAASGFIFSSYAMHWVRQAPGNGLEWVA
FMSYDGSNKKYADSVKGRFTISRDNSKNTLYLQMNSLRAEDTAVYYCAR
DRGIAAGGNYYYYGMDVWGQGTTVTVSSASTKGPSEVQLVESGGGLVQP
GGSLRLSCAASGYDFTHYGMNWVRQAPGKGLEWVGWINTYTGEPTYAAD
FKRRFTFSLDTSKSTAYLQMNSLRAEDTAVYYCAKYPYYYGTSHWYFDV
WGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNH
KPSNTKVDKKVEPKSCDKTHL
SEQ ID NO: 103
EIVLTQSPATLSLSPGERATLSCRASQSVYSYLAWYQQKPGQAPRLLIY
DASNRATGIPARFSGSGSGTDFTLTISSLEPEDFAVYYCQQRSNWPPFT
FGPGTKVDIKRTVAAPSDIQLTQSPSSLSASVGDRVTITCSASQDISNY
LNWYQQKPGKAPKVLIYFTSSLHSGVPSRFSGSGSGTDFTLTISSLQPE
DFATYYCQQYSTVPWTFGQGTKVEIKRTVAAPSVFIFPPSDEQLKSGTA
SVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 104
QVQLVESGGGVVQPGRSLRLSCAASGFIFSSYAMHWVRQAPGNGLEWVA
FMSYDGSNKKYADSVKGRFTISRDNSKNTLYLQMNSLRAEDTAVYYCAR
DRGIAAGGNYYYYGMDVWGQGTTVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGKTVAAPSEVQLLVSGGGLVQPGGSLRLSCAASGFT
FKAYPMMWVRQAPGKGLEWVSEISPSGSYTYYADSVKGRFTISRDNSKN
TLYLQMNSLRAEDTAVYYCAKDPRKLDYWGQGTLVTVSS
SEQ ID NO: 105
QVQLVESGGGVVQPGRSLRLSCAASGFIFSSYAMHWVRQAPGNGLEWVA
FMSYDGSNKKYADSVKGRFTISRDNSKNTLYLQMNSLRAEDTAVYYCAR
DRGIAAGGNYYYYGMDVWGQGTTVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGKTVAAPSDIQMTQSPSSLSASVGDRVTITCRASQW
IGPELRWYQQKPGKAPKLLIYHTSILQSGVPSRFSGSGSGTDFTLTISS
LQPEDFATYYCQQYMFQPMTFGQGTKVEIKR
SEQ ID NO: 106
QVQLVESGGGVVQPGRSLRLSCAASGFIFSSYAMHWVRQAPGNGLEWVA
FMSYDGSNKKYADSVKGRFTISRDNSKNTLYLQMNSLRAEDTAVYYCAR
DRGIAAGGNYYYYGMDVWGQGTTVTVSSASTKGPSVFPLAPSSKSTSGG
TAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGKTVAAPSDGGGIRRSMSGTWYLKAMTVDREFPEMN
LESVTPMTLTLLKGHNLEAKVTMLISGRCQEVKAVLGRTKERKKYTADG
GKHVAYIIPSAVRDHVIFYSEGQLHGKPVRGVKLVGRDPKNNLEALEDF
EKAAGARGLSTESILIPRQSETCSPG
SEQ ID NO: 107
EIVLTQSPATLSLSPGERATLSCRASQSVYSYLAWYQQKPGQAPRLLIY
DASNRATGIPARFSGSGSGTDFTLTISSLEPEDFAVYYCQQRSNWPPFT
FGPGTKVDIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGECTVAAPSEVQLLVSGGGLVQPGGSLRLSCAA
SGFTFKAYPMMWVRQAPGKGLEWVSEISPSGSYTYYADSVKGRFTISRD
NSKNTLYLQMNSLRAEDTAVYYCAKDPRKLDYWGQGTLVTVSS
SEQ ID NO: 108
EIVLTQSPATLSLSPGERATLSCRASQSVYSYLAWYQQKPGQAPRLLIY
DASNRATGIPARFSGSGSGTDFTLTISSLEPEDFAVYYCQQRSNWPPFT
FGPGTKVDIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGECTVAAPSDIQMTQSPSSLSASVGDRVTITCR
ASQWIGPELRWYQQKPGKAPKLLIYHTSILQSGVPSRFSGSGSGTDFTL
TISSLQPEDFATYYCQQYMFQPMTFGQGTKVEIKR
SEQ ID NO: 109
EIVLTQSPATLSLSPGERATLSCRASQSVYSYLAWYQQKPGQAPRLLIY
DASNRATGIPARFSGSGSGTDFTLTISSLEPEDFAVYYCQQRSNWPPFT
FGPGTKVDIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGECTVAAPSDGGGIRRSMSGTWYLKAMTVDREF
PEMNLESVTPMTLTLLKGHNLEAKVTMLISGRCQEVKAVLGRTKERKKY
TADGGKHVAYIIPSAVRDHVIFYSEGQLHGKPVRGVKLVGRDPKNNLEA
LEDFEKAAGARGLSTESILIPRQSETCSPG
SEQ ID NO: 110
QVQLVQSGAEVKKPGASVKVSCTASGYTFTHYGMNWVRQAPGKGLEWMG
WINTYTGEPTYADKFKDRFTFSLETSASTVYMELTSLTSDDTAVYYCAR
ERGDAMDYWGQGTLVTVSSASTKGPSEVQLVESGGGLVQPGGSLRLSCA
ASGYTFTNYGMNWVRQAPGKGLEWVGWINTYTGEPTYAADFKRRFTFSL
DTSKSTAYLQMNSLRAEDTAVYYCAKYPHYYGSSHWYFDVWGQGTLVTV
SSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 111
DIVMTQSPSSLSASVGDRVTLTCTASQSVSNDVVWYQQRPGKAPKLLIY
SAFNRYTGVPSRFSGRGYGTDFTLTISSLQPEDVAVYYCQQDYNSPRTF
GQGTKLEVKRTVAAPSDIQMTQSPSSLSASVGDRVTITCSASQDISNYL
NWYQQKPGKAPKVLIYFTSSLHSGVPSRFSGSGSGTDFTLTISSLQPED
FATYYCQQYSTVPWTFGQGTKVEIKRTVAAPSVFIFPPSDEQLKSGTAS
VVCLLNNFYPREAKVQWKVDNALQSGNSQESVTEQDSKDSTYSLSSTLT
LSKADYEKHKVYACEVTHQGLSSPVTKSFNRGEC
SEQ ID NO: 112
QVQLVQSGAEVKKPGASVKVSCTASGYTFTHYGMNWVRQAPGKGLEWMG
WINTYTGEPTYADKFKDRFTFSLETSASTVYMELTSLTSDDTAVYYCAR
ERGDAMDYWGQGTLVTVSSASTKGPSQVQLVQSGAEVKKPGASVKVSCK
ASGGTFSSYAISWVRQAPGQGLEWMGGFDPEDGETIYAQKFQGRVTMTE
DTSTDTAYMELSSLRSEDTAVYYCATGRSMFRGVIIPFNGMDVWGQGTT
VTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 113
DIVMTQSPSSLSASVGDRVTLTCTASQSVSNDVVWYQQRPGKAPKLLIY
SAFNRYTGVPSRFSGRGYGTDFTLTISSLQPEDVAVYYCQQDYNSPRTF
GQGTKLEVKRTVAAPSEIVLTQSPATLSLSPGERATLSCRASQSVYSYL
AWYQQKPGQAPRLLIYDASNRATGIPARFSGSGSGTDFTLTISSLEPED
FAVYYCQQRSNWPPFTFGPGTKVDIKRTVAAPSDIRMTQSPSSLSASVG
DRVTITCRASQSISSYLNWYQQKPGKAPKLLIYAASSLQSGVPSRFSGS
GSGTDFTLTISSLQPEDFATYYCQQSYSTPLTFGGGTKVEIKRTVAAPS
VFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRG
EC
SEQ ID NO: 114
QVQLVQSGAEVKKPGASVKVSCTASGYTFTHYGMNWVRQAPGKGLEWMG
WINTYTGEPTYADKFKDRFTFSLETSASTVYMELTSLTSDDTAVYYCAR
ERGDAMDYWGQGTLVTVSSASTKGPSEVQLVESGGGLVQPGGSLRLSCA
ASGYDFTHYGMNWVRQAPGKGLEWVGWINTYTGEPTYAADFKRRFTFSL
DTSKSTAYLQMNSLRAEDTAVYYCAKYPYYYGTSHWYFDVWGQGTLVTV
SSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDK
KVEPKSCDKTHL
SEQ ID NO: 115
DIVMTQSPSSLSASVGDRVTLTCTASQSVSNDVVWYQQRPGKAPKLLIY
SAFNRYTGVPSRFSGRGYGTDFTLTISSLQPEDVAVYYCQQDYNSPRTF
GQGTKLEVKRTVAAPSDIQLTQSPSSLSASVGDRVTITCSASQDISNYL
NWYQQKPGKAPKVLIYFTSSLHSGVPSRFSGSGSGTDFTLTISSLQPED
FATYYCQQYSTVPWTFGQGTKVEIKRTVAAPSVFIFPPSDEQLKSGTAS
VVCLLNNFYPREAKVQWKVDNALQSGNSQESVTEQDSKDSTYSLSSTLT
LSKADYEKHKVYACEVTHQGLSSPVTKSFNRGEC
SEQ ID NO: 116
DIVMTQSPSSLSASVGDRVTLTCTASQSVSNDVVWYQQRPGKAPKLLIY
SAFNRYTGVPSRFSGRGYGTDFTLTISSLQPEDVAVYYCQQDYNSPRTF
GQGTKLEVKRGGGGSGGGGSGGGGSSGGGSQVQLVQSGAEVKKPGASVK
VSCTASGYTFTHYGMNWVRQAPGKGLEWMGWINTYTGEPTYADKFKDRF
TFSLETSASTVYMELTSLTSDDTAVYYCARERGDAMDYWGQGTLVTVSS
ASTKGPSEVQLLVSGGGLVQPGGSLRLSCAASGFTFKAYPMMWVRQAPG
KGLEWVSEISPSGSYTYYADSVKGRFTISRDNSKNTLYLQMNSLRAEDT
AVYYCAKDPRKLDYWGQGTLVTVSS
SEQ ID NO: 117
DIVMTQSPSSLSASVGDRVTLTCTASQSVSNDVVWYQQRPGKAPKLLIY
SAFNRYTGVPSRFSGRGYGTDFTLTISSLQPEDVAVYYCQQDYNSPRTF
GQGTKLEVKRGGGGSGGGGSGGGGSSGGGSQVQLVQSGAEVKKPGASVK
VSCTASGYTFTHYGMNWVRQAPGKGLEWMGWINTYTGEPTYADKFKDRF
TFSLETSASTVYMELTSLTSDDTAVYYCARERGDAMDYWGQGTLVTVSS
ASTKGPSDIQMTQSPSSLSASVGDRVTITCRASQWIGPELRWYQQKPGK
APKLLIYHTSILQSGVPSRFSGSGSGTDFTLTISSLQPEDFATYYCQQY
MFQPMTFGQGTKVEIKR
SEQ ID NO: 118
DIVMTQSPSSLSASVGDRVTLTCTASQSVSNDVVWYQQRPGKAPKLLIY
SAFNRYTGVPSRFSGRGYGTDFTLTISSLQPEDVAVYYCQQDYNSPRTF
GQGTKLEVKRGGGGSGGGGSGGGGSSGGGSQVQLVQSGAEVKKPGASVK
VSCTASGYTFTHYGMNWVRQAPGKGLEWMGWINTYTGEPTYADKFKDRF
TFSLETSASTVYMELTSLTSDDTAVYYCARERGDAMDYWGQGTLVTVSS
ASTKGPSDGGGIRRSMSGTWYLKAMTVDREFPEMNLESVTPMTLTLLKG
HNLEAKVTMLISGRCQEVKAVLGRTKERKKYTADGGKHVAYIIPSAVRD
HVIFYSEGQLHGKPVRGVKLVGRDPKNNLEALEDFEKAAGARGLSTESI
LIPRQSETCSPG
SEQ ID NO: 119
DIQMTQSPSSLSASVGDRVTITCRASQSIDSYLHWYQQKPGKAPKLLIY
SASELQSGVPSRFSGSGSGTDFTLTISSLQPEDFATYYCQQVVWRPFTF
GQGTKVEIKRTVAAPSDIQMTQSPSSLSASVGDRVTITCRASQWIGPEL
RWYQQKPGKAPKLLIYHTSILQSGVPSRFSGSGSGTDFTLTISSLQPED
FATYYCQQYMFQPMTFGQGTKVEIKR
SEQ ID NO: 120
DIQMTQSPSSLSASVGDRVTITCRASQSIDSYLHWYQQKPGKAPKLLIY
SASELQSGVPSRFSGSGSGTDFTLTISSLQPEDFATYYCQQVVWRPFTF
GQGTKVEIKRTVAAPSDGGGIRRSMSGTWYLKAMTVDREFPEMNLESVT
PMTLTLLKGHNLEAKVTMLISGRCQEVKAVLGRTKERKKYTADGGKHVA
YIIPSAVRDHVIFYSEGQLHGKPVRGVKLVGRDPKNNLEALEDFEKAAG
ARGLSTESILIPRQSETCSPG
SEQ ID NO: 121
VSDVPRDLEVVAATPTSLLISWDTHNAYNGYYRITYGETGGNSPVREFT
VPHPEVTATISGLKPGVDDTITVYAVTNHHMPLRIFGPISINHRTTVAA
PSEVQLLVSGGGLVQPGGSLRLSCAASGFTFKAYPMMWVRQAPGKGLEW
VSEISPSGSYTYYADSVKGRFTISRDNSKNTLYLQMNSLRAEDTAVYYC
AKDPRKLDYWGQGTLVTVSS
SEQ ID NO: 122
VSDVPRDLEVVAATPTSLLISWDTHNAYNGYYRITYGETGGNSPVREFT
VPHPEVTATISGLKPGVDDTITVYAVTNHHMPLRIFGPISINHRTTVAA
PSDIQMTQSPSSLSASVGDRVTITCRASQWIGPELRWYQQKPGKAPKLL
IYHTSILQSGVPSRFSGSGSGTDFTLTISSLQPEDFATYYCQQYMFQPM
TFGQGTKVEIKR
SEQ ID NO: 123
VSDVPRDLEVVAATPTSLLISWDTHNAYNGYYRITYGETGGNSPVREFT
VPHPEVTATISGLKPGVDDTITVYAVTNHHMPLRIFGPISINHRTTVAA
PSDGGGIRRSMSGTWYLKAMTVDREFPEMNLESVTPMTLTLLKGHNLEA
KVTMLISGRCQEVKAVLGRTKERKKYTADGGKHVAYIIPSAVRDHVIFY
SEGQLHGKPVRGVKLVGRDPKNNLEALEDFEKAAGARGLSTESILIPRQ
SETCSPG
SEQ ID NO: 124
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDVWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGKTVAAPSDIVMTQSPSSLSASVGDRVTLTCTASQSVSN
DVVWYQQRPGKAPKLLIYSAFNRYTGVPSRFSGRGYGTDFTLTISSLQP
EDVAVYYCQQDYNSPRTFGQGTKLEVKRGGGGSGGGGSGGGGSSGGGSQ
VQLVQSGAEVKKPGASVKVSCTASGYTFTHYGMNWVRQAPGKGLEWMGW
INTYTGEPTYADKFKDRFTFSLETSASTVYMELTSLTSDDTAVYYCARE
RGDAMDYWGQGTLVTVSS
SEQ ID NO: 125
DIQMTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSDIVMTQSPSSLSASVGDRVTLTCTA
SQSVSNDVVWYQQRPGKAPKLLIYSAFNRYTGVPSRFSGRGYGTDFTLT
ISSLQPEDVAVYYCQQDYNSPRTFGQGTKLEVKRGGGGSGGGGSGGGGS
SGGGSQVQLVQSGAEVKKPGASVKVSCTASGYTFTHYGMNWVRQAPGKG
LEWMGWINTYTGEPTYADKFKDRFTFSLETSASTVYMELTSLTSDDTAV
YYCARERGDAMDYWGQGTLVTVSS
SEQ ID NO: 126
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDVWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGKTVAAPSDIQMTQSPSSLSASVGDRVTITCRASQSIDS
YLHWYQQKPGKAPKLLIYSASELQSGVPSRFSGSGSGTDFTLTISSLQP
EDFATYYCQQVVWRPFTFGQGTKVEIKR
SEQ ID NO: 127
DIQMTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSDIQMTQSPSSLSASVGDRVTITCRA
SQSIDSYLHWYQQKPGKAPKLLIYSASELQSGVPSRFSGSGSGTDFTLT
ISSLQPEDFATYYCQQVVWRPFTFGQGTKVEIKR
SEQ ID NO: 128
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDVWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGKTVAAPSVSDVPRDLEVVAATPTSLLISWDTHNAYNGY
YRITYGETGGNSPVREFTVPHPEVTATISGLKPGVDDTITVYAVTNHHM
PLRIFGPISINHRT
SEQ ID NO: 129
DIQMTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECTVAAPSVSDVPRDLEVVAATPTSLLISWDTH
NAYNGYYRITYGETGGNSPVREFTVPHPEVTATISGLKPGVDDTITVYA
VTNHHMPLRIFGPISINHRT
SEQ ID NO: 130
SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEKDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGKTVAAPSDIV
MTQSPSSLSASVGDRVTLTCTASQSVSNDVVWYQQRPGKAPKLLIYSAF
NRYTGVPSRFSGRGYGTDFTLTISSLQPEDVAVYYCQQDYNSPRTFGQG
TKLEVKRGGGGSGGGGSGGGGSSGGGSQVQLVQSGAEVKKPGASVKVSC
TASGYTFTHYGMNWVRQAPGKGLEWMGWINTYTGEPTYADKFKDRFTFS
LETSASTVYMELTSLTSDDTAVYYCARERGDAMDYWGQGTLVTVSS
SEQ ID NO: 131
SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEKDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGKTVAAPSDIQ
MTQSPSSLSASVGDRVTITCRASQSIDSYLHWYQQKPGKAPKLLIYSAS
ELQSGVPSRFSGSGSGTDFTLTISSLQPEDFATYYCQQVVWRPFTFGQG
TKVEIKR
SEQ ID NO: 132
SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEKDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGKTVAAPSVSD
VPRDLEVVAATPTSLLISWDTHNAYNGYYRITYGETGGNSPVREFTVPH
PEVTATISGLKPGVDDTITVYAVTNHHMPLRIFGPISINHRT
SEQ ID NO: 133
EVQLLVSGGGLVQPGGSLRLSCAASGFTFKAYPMMWVRQAPGKGLEWVS
EISPSGSYTYYADSVKGRFTISRDNSKNTLYLQMNSLRAEDTAVYYCAK
DPRKLDYWGQGTLVTVSSASTKGPSDIVMTQSPSSLSASVGDRVTLTCT
ASQSVSNDVVWYQQRPGKAPKLLIYSAFNRYTGVPSRFSGRGYGTDFTL
TISSLQPEDVAVYYCQQDYNSPRTFGQGTKLEVKRGGGGSGGGGSGGGG
SSGGGSQVQLVQSGAEVKKPGASVKVSCTASGYTFTHYGMNWVRQAPGK
GLEWMGWINTYTGEPTYADKFKDRFTFSLETSASTVYMELTSLTSDDTA
VYYCARERGDAMDYWGQGTLVTVSS
SEQ ID NO: 134
EVQLLVSGGGLVQPGGSLRLSCAASGFTFKAYPMMWVRQAPGKGLEWVS
EISPSGSYTYYADSVKGRFTISRDNSKNTLYLQMNSLRAEDTAVYYCAK
DPRKLDYWGQGTLVTVSSASTKGSPVSDVPRDLEVVAATPTSLLISWDT
HNAYNGYYRITYGETGGNSPVREFTVPHPEVTATISGLKPGVDDTITVY
AVTNHHMPLRIFGPISINHRT
SEQ ID NO: 135
DIQMTQSPSSLSASVGDRVTITCRASQWIGPELRWYQQKPGKAPKLLIY
HTSILQSGVPSRFSGSGSGTDFTLTISSLQPEDFATYYCQQYMFQPMTF
GQGTKVEIKRTVAAPSDIVMTQSPSSLSASVGDRVTLTCTASQSVSNDV
VWYQQRPGKAPKLLIYSAFNRYTGVPSRFSGRGYGTDFTLTISSLQPED
VAVYYCQQDYNSPRTFGQGTKLEVKRGGGGSGGGGSGGGGSSGGGSQVQ
LVQSGAEVKKPGASVKVSCTASGYTFTHYGMNWVRQAPGKGLEWMGWIN
TYTGEPTYADKFKDRFTFSLETSASTVYMELTSLTSDDTAVYYCARERG
DAMDYWGQGTLVTVSS
SEQ ID NO: 136
DIQMTQSPSSLSASVGDRVTITCRASQWIGPELRWYQQKPGKAPKLLIY
HTSILQSGVPSRFSGSGSGTDFTLTISSLQPEDFATYYCQQYMFQPMTF
GQGTKVEIKRTVAAPSDIQMTQSPSSLSASVGDRVTITCRASQSIDSYL
HWYQQKPGKAPKLLIYSASELQSGVPSRFSGSGSGTDFTLTISSLQPED
FATYYCQQVVWRPFTFGQGTKVEIKR
SEQ ID NO: 137
DIQMTQSPSSLSASVGDRVTITCRASQWIGPELRWYQQKPGKAPKLLIY
HTSILQSGVPSRFSGSGSGTDFTLTISSLQPEDFATYYCQQYMFQPMTF
GQGTKVEIKRTVAAPSVSDVPRDLEVVAATPTSLLISWDTHNAYNGYYR
ITYGETGGNSPVREFTVPHPEVTATISGLKPGVDDTITVYAVTNHHMPL
RIFGPISINHRT
SEQ ID NO: 138
DGGGIRRSMSGTWYLKAMTVDREFPEMNLESVTPMTLTLLKGHNLEAKV
TMLISGRCQEVKAVLGRTKERKKYTADGGKHVAYIIPSAVRDHVIFYSE
GQLHGKPVRGVKLVGRDPKNNLEALEDFEKAAGARGLSTESILIPRQSE
TCSPGTVAAPSDIVMTQSPSSLSASVGDRVTLTCTASQSVSNDVVWYQQ
RPGKAPKLLIYSAFNRYTGVPSRFSGRGYGTDFTLTISSLQPEDVAVYY
CQQDYNSPRTFGQGTKLEVKRGGGGSGGGGSGGGGSSGGGSQVQLVQSG
AEVKKPGASVKVSCTASGYTFTHYGMNWVRQAPGKGLEWMGWINTYTGE
PTYADKFKDRFTFSLETSASTVYMELTSLTSDDTAVYYCARERGDAMDY
WGQGTLVTVSS
SEQ ID NO: 139
DGGGIRRSMSGTWYLKAMTVDREFPEMNLESVTPMTLTLLKGHNLEAKV
TMLISGRCQEVKAVLGRTKERKKYTADGGKHVAYIIPSAVRDHVIFYSE
GQLHGKPVRGVKLVGRDPKNNLEALEDFEKAAGARGLSTESILIPRQSE
TCSPGTVAAPSDIQMTQSPSSLSASVGDRVTITCRASQSIDSYLHWYQQ
KPGKAPKLLIYSASELQSGVPSRFSGSGSGTDFTLTISSLQPEDFATYY
CQQVVWRPFTFGQGTKVEIKR
SEQ ID NO: 140
DGGGIRRSMSGTWYLKAMTVDREFPEMNLESVTPMTLTLLKGHNLEAKV
TMLISGRCQEVKAVLGRTKERKKYTADGGKHVAYIIPSAVRDHVIFYSE
GQLHGKPVRGVKLVGRDPKNNLEALEDFEKAAGARGLSTESILIPRQSE
TCSPGTVAAPSVSDVPRDLEVVAATPTSLLISWDTHNAYNGYYRITYGE
TGGNSPVREFTVPHPEVTATISGLKPGVDDTITVYAVTNHHMPLRIFGP
ISINHRT
SEQ ID NO: 141
GAGGTGCAGCTGGTGGAGTCTGGCGGCGGACTGGTGCAGCCCGGCAGAA
GCCTGAGACTGAGCTGTGCCGCCAGCGGCTTCACCTTCGACGACTACGC
CATGCACTGGGTGAGGCAGGCCCCTGGCAAGGGCCTGGAGTGGGTGTCC
GCCATCACCTGGAATAGCGGCCACATCGACTACGCCGACAGCGTGGAGG
GCAGATTCACCATCAGCCGGGACAACGCCAAGAACAGCCTGTACCTGCA
GATGAACAGCCTGAGAGCCGAGGACACCGCCGTGTACTACTGTGCCAAG
GTGTCCTACCTGAGCACCGCCAGCAGCCTGGACTACTGGGGCCAGGGCA
CCCTGGTGACAGTCTCGAGCGCTAGCACCAAGGGCCCCAGCGTGTTCCC
CCTGGCCCCCAGCAGCAAGAGCACCAGCGGCGGCACAGCCGCCCTGGGC
TGCCTGGTGAAGGACTACTTCCCCGAGCCTGTGACCGTGTCCTGGAATA
GCGGAGCCCTGACCTCCGGCGTGCACACCTTCCCCGCCGTGCTGCAGAG
CAGCGGCCTGTACTCCCTGAGCAGCGTGGTGACCGTGCCCAGCAGCAGC
CTGGGCACCCAGACCTACATCTGCAACGTGAACCACAAGCCCAGCAACA
CCAAAGTGGACAAGAAAGTGGAGCCCAAGAGCTGCGATAAGACCCACAC
CTGCCCCCCCTGCCCTGCCCCCGAGCTGGCCGGCGCCCCTAGCGTGTTC
CTGTTCCCCCCCAAGCCTAAGGACACCCTGATGATCAGCAGGACCCCCG
AAGTGACCTGCGTGGTGGTGGATGTGAGCCACGAGGACCCTGAAGTGAA
GTTCAACTGGTACGTGGACGGCGTGGAAGTGCACAACGCCAAGACCAAG
CCCAGAGAGGAGCAGTACAACAGCACCTACCGCGTGGTGTCTGTGCTGA
CCGTGCTGCACCAGGATTGGCTGAACGGCAAGGAGTACAAGTGCAAAGT
GAGCAACAAGGCCCTGCCTGCCCCTATCGAGAAAACCATCAGCAAGGCC
AAGGGCCAGCCTAGAGAGCCCCAGGTCTACACCCTGCCTCCCTCCAGAG
ATGAGCTGACCAAGAACCAGGTGTCCCTGACCTGTCTGGTGAAGGGCTT
CTACCCCAGCGACATCGCCGTGGAGTGGGAGAGCAACGGCCAGCCCGAG
AACAACTACAAGACCACCCCCCCTGTGCTGGACAGCGATGGCAGCTTCT
TCCTGTACTCCAAGCTGACCGTGGACAAGAGCAGATGGCAGCAGGGCAA
CGTGTTCAGCTGCAGCGTGATGCACGAGGCCCTGCACAATCACTACACC
CAGAAGAGTCTGAGCCTGTCCCCTGGCAAGGGATCCACCGTGGCCGCTC
CCAGCGGATCAGAGGTGCAGCTGCTGGTGTCTGGCGGCGGACTGGTGCA
GCCTGGCGGCAGCCTGAGACTGAGCTGCGCCGCCAGCGGCTTCACCTTC
AAGGCCTACCCCATGATGTGGGTGCGGCAGGCCCCTGGCAAGGGCCTGG
AATGGGTGTCCGAGATCAGCCCCAGCGGCAGCTACACCTACTACGCCGA
CAGCGTGAAGGGCCGGTTCACCATCAGCCGGGACAACAGCAAGAACACC
CTGTACCTGCAGATGAACAGCCTGCGGGCCGAGGACACCGCCGTGTACT
ACTGCGCCAAGGACCCCCGGAAGCTGGACTACTGGGGCCAGGGCACCCT
GGTGACCGTGAGCAGC
SEQ ID NO: 142
GAGGTGCAGCTGGTGGAGTCTGGCGGCGGACTGGTGCAGCCCGGCAGAA
GCCTGAGACTGAGCTGTGCCGCCAGCGGCTTCACCTTCGACGACTACGC
CATGCACTGGGTGAGGCAGGCCCCTGGCAAGGGCCTGGAGTGGGTGTCC
GCCATCACCTGGAATAGCGGCCACATCGACTACGCCGACAGCGTGGAGG
GCAGATTCACCATCAGCCGGGACAACGCCAAGAACAGCCTGTACCTGCA
GATGAACAGCCTGAGAGCCGAGGACACCGCCGTGTACTACTGTGCCAAG
GTGTCCTACCTGAGCACCGCCAGCAGCCTGGACTACTGGGGCCAGGGCA
CCCTGGTGACAGTCTCGAGCGCTAGCACCAAGGGCCCCAGCGTGTTCCC
CCTGGCCCCCAGCAGCAAGAGCACCAGCGGCGGCACAGCCGCCCTGGGC
TGCCTGGTGAAGGACTACTTCCCCGAGCCTGTGACCGTGTCCTGGAATA
GCGGAGCCCTGACCTCCGGCGTGCACACCTTCCCCGCCGTGCTGCAGAG
CAGCGGCCTGTACTCCCTGAGCAGCGTGGTGACCGTGCCCAGCAGCAGC
CTGGGCACCCAGACCTACATCTGCAACGTGAACCACAAGCCCAGCAACA
CCAAAGTGGACAAGAAAGTGGAGCCCAAGAGCTGCGATAAGACCCACAC
CTGCCCCCCCTGCCCTGCCCCCGAGCTGGCCGGCGCCCCTAGCGTGTTC
CTGTTCCCCCCCAAGCCTAAGGACACCCTGATGATCAGCAGGACCCCCG
AAGTGACCTGCGTGGTGGTGGATGTGAGCCACGAGGACCCTGAAGTGAA
GTTCAACTGGTACGTGGACGGCGTGGAAGTGCACAACGCCAAGACCAAG
CCCAGAGAGGAGCAGTACAACAGCACCTACCGCGTGGTGTCTGTGCTGA
CCGTGCTGCACCAGGATTGGCTGAACGGCAAGGAGTACAAGTGCAAAGT
GAGCAACAAGGCCCTGCCTGCCCCTATCGAGAAAACCATCAGCAAGGCC
AAGGGCCAGCCTAGAGAGCCCCAGGTCTACACCCTGCCTCCCTCCAGAG
ATGAGCTGACCAAGAACCAGGTGTCCCTGACCTGTCTGGTGAAGGGCTT
CTACCCCAGCGACATCGCCGTGGAGTGGGAGAGCAACGGCCAGCCCGAG
AACAACTACAAGACCACCCCCCCTGTGCTGGACAGCGATGGCAGCTTCT
TCCTGTACTCCAAGCTGACCGTGGACAAGAGCAGATGGCAGCAGGGCAA
CGTGTTCAGCTGCAGCGTGATGCACGAGGCCCTGCACAATCACTACACC
CAGAAGAGTCTGAGCCTGTCCCCTGGCAAGGGATCCACAGTGGCTGCAC
CTTCCGGGTCAACCGTCGCCGCCCCCAGCGGAAGCGAGGTGCAGCTGCT
GGTGTCTGGCGGCGGACTGGTGCAGCCTGGCGGCAGCCTGAGACTGAGC
TGCGCCGCCAGCGGCTTCACCTTCAAGGCCTACCCCATGATGTGGGTGC
GGCAGGCCCCTGGCAAGGGCCTGGAATGGGTGTCCGAGATCAGCCCCAG
CGGCAGCTACACCTACTACGCCGACAGCGTGAAGGGCCGGTTCACCATC
AGCCGGGACAACAGCAAGAACACCCTGTACCTGCAGATGAACAGCCTGC
GGGCCGAGGACACCGCCGTGTACTACTGCGCCAAGGACCCCCGGAAGCT
GGACTACTGGGGCCAGGGCACCCTGGTGACCGTGAGCAGC
SEQ ID NO: 143
GAGGTGCAGCTGGTGGAGTCTGGCGGCGGACTGGTGCAGCCCGGCAGAA
GCCTGAGACTGAGCTGTGCCGCCAGCGGCTTCACCTTCGACGACTACGC
CATGCACTGGGTGAGGCAGGCCCCTGGCAAGGGCCTGGAGTGGGTGTCC
GCCATCACCTGGAATAGCGGCCACATCGACTACGCCGACAGCGTGGAGG
GCAGATTCACCATCAGCCGGGACAACGCCAAGAACAGCCTGTACCTGCA
GATGAACAGCCTGAGAGCCGAGGACACCGCCGTGTACTACTGTGCCAAG
GTGTCCTACCTGAGCACCGCCAGCAGCCTGGACTACTGGGGCCAGGGCA
CCCTGGTGACAGTCTCGAGCGCTAGCACCAAGGGCCCCAGCGTGTTCCC
CCTGGCCCCCAGCAGCAAGAGCACCAGCGGCGGCACAGCCGCCCTGGGC
TGCCTGGTGAAGGACTACTTCCCCGAGCCTGTGACCGTGTCCTGGAATA
GCGGAGCCCTGACCTCCGGCGTGCACACCTTCCCCGCCGTGCTGCAGAG
CAGCGGCCTGTACTCCCTGAGCAGCGTGGTGACCGTGCCCAGCAGCAGC
CTGGGCACCCAGACCTACATCTGCAACGTGAACCACAAGCCCAGCAACA
CCAAAGTGGACAAGAAAGTGGAGCCCAAGAGCTGCGATAAGACCCACAC
CTGCCCCCCCTGCCCTGCCCCCGAGCTGGCCGGCGCCCCTAGCGTGTTC
CTGTTCCCCCCCAAGCCTAAGGACACCCTGATGATCAGCAGGACCCCCG
AAGTGACCTGCGTGGTGGTGGATGTGAGCCACGAGGACCCTGAAGTGAA
GTTCAACTGGTACGTGGACGGCGTGGAAGTGCACAACGCCAAGACCAAG
CCCAGAGAGGAGCAGTACAACAGCACCTACCGCGTGGTGTCTGTGCTGA
CCGTGCTGCACCAGGATTGGCTGAACGGCAAGGAGTACAAGTGCAAAGT
GAGCAACAAGGCCCTGCCTGCCCCTATCGAGAAAACCATCAGCAAGGCC
AAGGGCCAGCCTAGAGAGCCCCAGGTCTACACCCTGCCTCCCTCCAGAG
ATGAGCTGACCAAGAACCAGGTGTCCCTGACCTGTCTGGTGAAGGGCTT
CTACCCCAGCGACATCGCCGTGGAGTGGGAGAGCAACGGCCAGCCCGAG
AACAACTACAAGACCACCCCCCCTGTGCTGGACAGCGATGGCAGCTTCT
TCCTGTACTCCAAGCTGACCGTGGACAAGAGCAGATGGCAGCAGGGCAA
CGTGTTCAGCTGCAGCGTGATGCACGAGGCCCTGCACAATCACTACACC
CAGAAGAGTCTGAGCCTGTCCCCTGGCAAGGGATCCACCGTCGCCGCAC
CAAGCGGGTCAACAGTGGCCGCTCCCTCCGGCAGCACTGTGGCTGCCCC
CAGCGGAAGCGAGGTGCAGCTGCTGGTGTCTGGCGGCGGACTGGTGCAG
CCTGGCGGCAGCCTGAGACTGAGCTGCGCCGCCAGCGGCTTCACCTTCA
AGGCCTACCCCATGATGTGGGTGCGGCAGGCCCCTGGCAAGGGCCTGGA
ATGGGTGTCCGAGATCAGCCCCAGCGGCAGCTACACCTACTACGCCGAC
AGCGTGAAGGGCCGGTTCACCATCAGCCGGGACAACAGCAAGAACACCC
TGTACCTGCAGATGAACAGCCTGCGGGCCGAGGACACCGCCGTGTACTA
CTGCGCCAAGGACCCCCGGAAGCTGGACTACTGGGGCCAGGGCACCCTG
GTGACCGTGAGCAGC
SEQ ID NO: 144
GAGGTGCAGCTGGTGGAGTCTGGCGGCGGACTGGTGCAGCCCGGCAGAA
GCCTGAGACTGAGCTGTGCCGCCAGCGGCTTCACCTTCGACGACTACGC
CATGCACTGGGTGAGGCAGGCCCCTGGCAAGGGCCTGGAGTGGGTGTCC
GCCATCACCTGGAATAGCGGCCACATCGACTACGCCGACAGCGTGGAGG
GCAGATTCACCATCAGCCGGGACAACGCCAAGAACAGCCTGTACCTGCA
GATGAACAGCCTGAGAGCCGAGGACACCGCCGTGTACTACTGTGCCAAG
GTGTCCTACCTGAGCACCGCCAGCAGCCTGGACTACTGGGGCCAGGGCA
CCCTGGTGACAGTCTCGAGCGCTAGCACCAAGGGCCCCAGCGTGTTCCC
CCTGGCCCCCAGCAGCAAGAGCACCAGCGGCGGCACAGCCGCCCTGGGC
TGCCTGGTGAAGGACTACTTCCCCGAGCCTGTGACCGTGTCCTGGAATA
GCGGAGCCCTGACCTCCGGCGTGCACACCTTCCCCGCCGTGCTGCAGAG
CAGCGGCCTGTACTCCCTGAGCAGCGTGGTGACCGTGCCCAGCAGCAGC
CTGGGCACCCAGACCTACATCTGCAACGTGAACCACAAGCCCAGCAACA
CCAAAGTGGACAAGAAAGTGGAGCCCAAGAGCTGCGATAAGACCCACAC
CTGCCCCCCCTGCCCTGCCCCCGAGCTGGCCGGCGCCCCTAGCGTGTTC
CTGTTCCCCCCCAAGCCTAAGGACACCCTGATGATCAGCAGGACCCCCG
AAGTGACCTGCGTGGTGGTGGATGTGAGCCACGAGGACCCTGAAGTGAA
GTTCAACTGGTACGTGGACGGCGTGGAAGTGCACAACGCCAAGACCAAG
CCCAGAGAGGAGCAGTACAACAGCACCTACCGCGTGGTGTCTGTGCTGA
CCGTGCTGCACCAGGATTGGCTGAACGGCAAGGAGTACAAGTGCAAAGT
GAGCAACAAGGCCCTGCCTGCCCCTATCGAGAAAACCATCAGCAAGGCC
AAGGGCCAGCCTAGAGAGCCCCAGGTCTACACCCTGCCTCCCTCCAGAG
ATGAGCTGACCAAGAACCAGGTGTCCCTGACCTGTCTGGTGAAGGGCTT
CTACCCCAGCGACATCGCCGTGGAGTGGGAGAGCAACGGCCAGCCCGAG
AACAACTACAAGACCACCCCCCCTGTGCTGGACAGCGATGGCAGCTTCT
TCCTGTACTCCAAGCTGACCGTGGACAAGAGCAGATGGCAGCAGGGCAA
CGTGTTCAGCTGCAGCGTGATGCACGAGGCCCTGCACAATCACTACACC
CAGAAGAGTCTGAGCCTGTCCCCTGGCAAGGGATCCACCGTCGCCGCAC
CAAGCGGATCTACCGTCGCAGCCCCTTCCGGGTCAACAGTGGCCGCTCC
CTCCGGCAGCACTGTGGCTGCCCCCAGCGGAAGCGAGGTGCAGCTGCTG
GTGTCTGGCGGCGGACTGGTGCAGCCTGGCGGCAGCCTGAGACTGAGCT
GCGCCGCCAGCGGCTTCACCTTCAAGGCCTACCCCATGATGTGGGTGCG
GCAGGCCCCTGGCAAGGGCCTGGAATGGGTGTCCGAGATCAGCCCCAGC
GGCAGCTACACCTACTACGCCGACAGCGTGAAGGGCCGGTTCACCATCA
GCCGGGACAACAGCAAGAACACCCTGTACCTGCAGATGAACAGCCTGCG
GGCCGAGGACACCGCCGTGTACTACTGCGCCAAGGACCCCCGGAAGCTG
GACTACTGGGGCCAGGGCACCCTGGTGACCGTGAGCAGC
SEQ ID NO: 145
PASGS
SEQ ID NO: 146
PASPASGS
SEQ ID NO: 147
PASPASPASGS
SEQ ID NO: 148
GGGGSGGGGS
SEQ ID NO: 149
GGGGSGGGGSGGGGS
SEQ ID NO: 150
TVAAPSTVAAPSGS
SEQ ID NO: 151
TVAAPSTVAAPSTVAAPSGS
SEQ ID NO: 152
GSTVAAPSGSTVAAPSGSTVAAPSGSTVAAPSGSTVAAPSGS
SEQ ID NO: 153
GSTVAAPSGSTVAAPSGSTVAAPSGSTVAAPSGSTVAAPSGSTVAAPSG
S
SEQ ID NO: 154
PAVPPPGS
SEQ ID NO: 155
PAVPPPPAVPPPGS
SEQ ID NO: 156
PAVPPPPAVPPPPAVPPPGS
SEQ ID NO: 157
TVSDVPGS
SEQ ID NO: 158
TVSDVPTVSDVPGS
SEQ ID NO: 159
TVSDVPTVSDVPTVSDVPGS
SEQ ID NO: 160
TGLDSPGS
SEQ ID NO: 161
TGLDSPTGLDSPGS
SEQ ID NO: 162
TGLDSPTGLDSPTGLDSPGS
SEQ ID NO: 163
QVQLVQSGAEVKKPGASVKVSCKASGYTFTDYYMNWVRQAPGQGLEWMG
NINPNNGGTNYNQKFKDRVTMTTDTSTSTAYMELRSLRSDDTAVYYCAR
WILYYGRSKWYFDVWGRGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGKGSEVVAATPTSLLISWRHPHFPTRYYRITYGETGGNS
PVQEFTVPLQPPTATISGLKPGVDYTITVYAVTDGRNGRLLSIPISINY
RT
SEQ ID NO: 164
DIVMTQSPLSLPVTPGEPASISCRSSQSIVQSNGDTYLEWYLQKPGQSP
QLLIYRVSNRFSGVPDRFSGSGSGTDFTLKISRVEAEDVGVYYCFQGSH
VPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
SEQ ID NO: 165
MHVAQPAVVLASSRGIASFVCEYASPGKATEVRVTVLRQADSQVTEVCA
ATYMMGNELTFLDDSICTGTSSGNQVNLTIQGLRAMDTGLYICKVELMY
PPPYYLGIGNGTQIYVIDPEPCPDSDQEPKSSDKTHTSPPSPAPELLGG
SSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALH
NHYTQKSLSLSPGKGSGVQLLESGGGLVQPGGSLRLSCAASGFVFPWYD
MGWVRQAPGKGLEWVSSIDWHGKITYYADSVKGRFTISRDNSKNTLYLQ
MNSLRAEDTAVYYCATAEDEPGYDYWGQGTLVTVSS

1. A composition comprising a TNFα antagonist and a VEGF antagonist for use in preventing or treating a disease of the eye.

2. The composition of claim 1, wherein the TNFα antagonist and the VEGF antagonist are antigen binding proteins.

3. The composition of claim 1 or claim 2, wherein the TNFα antagonist and the VEGF antagonist are present in the form of a dual targeting protein.

4. The composition of claim 3, wherein the dual targeting protein comprises at least one paired VH/VL domain which binds TNFα or a TNFα receptor, and at least one paired VH/VL domain which binds VEGF or a VEGF receptor.

5. The composition of claim 4, wherein the dual targeting molecule is a DVD-Ig.

6. The composition of claim 3 or 4, wherein the dual targeting protein is a bispecific antibody.

7. The composition of claim 3, wherein the dual targeting protein is a dAb-dAb in-line fusion.

8. The composition of claim 3, wherein the dual targeting protein is a receptor-Fc fusion which is linked to one or more epitope binding domains.

9. The composition of claim 2, wherein the TNFα antagonist is an anti-TNFα antibody.

10. The composition of claim 2, wherein the VEGF antagonist is an anti-VEGF antibody.

11. The composition of claim 3, wherein the TNFα antagonist portion of the dual targeting protein is an anti-TNF antibody and wherein the VEGF antagonist portion of the dual targeting protein is an anti-VEGF epitope binding domain.

12. The composition of claim 3, wherein the VEGF antagonist portion of the dual targeting protein is an anti-VEGF antibody and the TNFα antagonist portion of the dual targeting protein is an anti-TNF epitope binding domain.

13. The composition of claim 8, 11 or 12, wherein the epitope binding domain is a dAb.

14. The composition of claim 13, wherein the dAb is a human dAb.

15. The composition of claim 8, 11 or 12, wherein the epitope binding domain is derived from a non-Ig scaffold.

16. The composition of claim 15 wherein the epitope binding domain is selected from CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin).

17. The composition according to claim 8 or any one of claims 11 to 16, wherein the epitope binding domain is directly attached to the antigen binding protein with a linker consisting of from 1 to 30 amino acids.

18. The composition according to claim 17, wherein the linker is selected from those set out in SEQ ID NO: 3-8 and 25, or any combination or multiple thereof.

19. The composition according to any one of claims 11 to 18, wherein the epitope binding domain is linked to the N-terminus of the antigen binding protein heavy chain.

20. The composition according to any one of claims 11 to 18, wherein the epitope binding domain is linked to the N-terminus of the antigen binding protein light chain.

21. The composition according to any one of claims 11 to 18, wherein the epitope binding domain is linked to the C-terminus of the antigen binding protein heavy chain.

22. The composition according to any one of claims 11 to 18, wherein the epitope binding domain is linked to the C-terminus of the antigen binding protein light chain.

23. A composition according to any one of claims 2 to 7, or 8 to 22, wherein the antigen binding protein comprises the CDRH1, CDRH2 and CDRH3 contained in the heavy chain set out in SEQ ID NO:10 and the CDRL1, CDRL2 and CDRL3 contained in the light chain set out in SEQ ID NO:12.

24. The composition according to claim 23 which comprises the heavy chain sequence of SEQ ID NO:14, 15, 47, 69, 70, 71 or 72 and the light chain sequence of SEQ ID NO:12.

25. The composition as claimed in any one of claims 3-8 or 11-24, wherein the composition is to be administered intravitreally every 4-6 weeks.

26. The composition as claimed in any one of claims 1-25, wherein the composition comprises a further active agent, optionally an anti-inflammatory agent.

27. Use of a composition as defined in any one of claims 1-26 for the manufacture of a medicament for use in preventing or treating a disease of the eye.

28. A TNFα antagonist selected from the group consisting of adalimumab, infliximab, etanercept, ESBA105, PEP1-5-19, PEP1-5-490, PEP1-5-493, an adnectin of SEQ ID NO:2, golimumab, certolizumab, ALK-6931, and an antibody comprising a heavy chain of SEQ ID NO:30 and a light chain of SEQ ID NO:31, for use in preventing or treating an eye disease, wherein the TNFα antagonist is to be administered in combination with a VEGF antagonist selected from the group consisting of bevacizumab, ranibizumab, r84, aflibercept, CT01, DOM15-10-11, DOM15-26-593, PRS-050, PRS-051, MP0012, CT-322, ESBA903, EPI-0030, EPI-0010, and DMS1571.

29. A VEGF antagonist selected from the group consisting of bevacizumab, ranibizumab, r84, aflibercept, CT01, DOM15-10-11, DOM15-26-593, PRS-050, PRS-051, MP0012, CT-322, ESBA903, EPI-0030, EPI-0010 and DMS1571, for use in preventing or treating an eye disease, wherein the VEGF antagonist is to be administered in combination with a TNFα antagonist selected from the group consisting of adalimumab, infliximab, etanercept, ESBA105, PEP1-5-19, PEP1-5-490, PEP1-5-493, an adnectin of SEQ ID NO:2, golimumab, certolizumab, ALK-6931, and an antibody comprising a heavy chain of SEQ ID NO:30 and a light chain of SEQ ID NO:31

30. A TNFα antagonist as claimed in claim 28 or the VEGF antagonist as claimed in claim 29, wherein the TNFα antagonist is adalimumab and the VEGF antagonist is ranibizumab.

31. A pharmaceutical composition comprising a composition as claimed in any one of claims 1 to 24 and a pharmaceutically acceptable carrier.

32. A pharmaceutical compositions as claimed in claim 31, wherein the composition comprises a further active agent, optionally an anti-inflammatory agent.

33. A polynucleotide sequence encoding an antigen binding protein as claimed in any one of claims 2 to 24.

34. A polynucleotide sequence encoding a heavy chain or light chain of a composition according to any one of claims 5, 6 or 9 to 24.

35. A polynucleotide sequence as claimed in claim 34, wherein the sequence is as set forth in SEQ ID NO: 11, 13, or 46.

36. A recombinant transformed or transfected host cell comprising one or more polynucleotide sequences as claimed in any one of claims 33-35.

37. A method for the production of a composition according to any one of claims 2 to 24 which method comprises the step of culturing a host cell of claim 36 and isolating the antigen binding protein.

38. A composition as claimed in any one of claims 1 to 24, which is for delivery via the intravitreal route.

39. A composition as claimed in any one of claims 1 to 24, which is for delivery via the periocular route.

40. A composition according to claim 39 which is for delivery via trans-scleral, subconjunctival, sub-tenon, peribulbar, topical, retrobulbar route or which is for delivery to the inferior, superior or lateral rectus muscle.

41. A composition according to any one of claims 1 to 24 wherein the disease of the eye is diabetic macula edema, cystoid macula edema, uveitis, AMD (Age related macular degeneration), choroidal neovascular AMD, diabetic retinopathy, retinal vein occlusion and other maculopathies and ocular vasculopathies.

42. A method of preventing or treating a patient afflicted with an eye disease comprising administering a prophylactically or therapeutically effective amount of a composition or dual targeting protein according to any one of claims 1 to 24 systemically or topically to the eye of the patient.

43. The method of claim 42, wherein said patient is suffering from at least one of the following diseases or disorders: diabetic macula edema, cystoid macula edema, uveitis, AMD (Age related macular degeneration), choroidal neovascular AMD, diabetic retinopathy, retinal vein occlusion and other maculopathies and ocular vasculopathies.

44. A dual targeting antigen binding molecule comprising a TNFα antagonist portion, a VEGF antagonist portion and a linker connecting said TNFα antagonist portion to said VEGF antagonist portion, wherein:
the TNFα antagonist portion comprises an amino acid sequence of any one of the TNFα antagonists listed in table 1;
the VEGF antagonist portion comprises an amino acid sequence of any one of the VEGF antagonists listed in table 2;
the linker is an amino acid sequence from 1-150 amino acids in length; and
the dual targeting molecule is not DMS4000 or DMS4031.

45. A dual targeting antigen binding molecule comprising a TNFα antagonist portion, a VEGF antagonist portion and a linker connecting said TNFα antagonist portion to said VEGF antagonist portion, wherein:
the TNFα antagonist portion comprises an amino acid sequence of any one of the TNFα antagonists listed in table 1;
the VEGF antagonist portion comprises an amino acid sequence of any one of the VEGF antagonists listed in table 2;
the linker is an amino acid sequence from 1-150 amino acids in length; and
wherein the dual targeting antigen binding molecule is for use in preventing or treating a disease of the eye and is to be administered intravitreally every 4-6 weeks.

46. A dual targeting molecule as claimed in claim 44 or 45, wherein the linker is selected from those set out in SEQ ID NO: 3-8, 25, 66-68, and 145-162 or any combination or multiple thereof.

47. A dual targeting antigen binding molecule as claimed in any one of claims 44-46, consisting of an amino acid sequence of SEQ ID NO:62 or SEQ ID NO 64.

48. An antigen binding protein comprising the heavy chain sequence of SEQ ID NO:69, 70, 71 or 72 and the light chain sequence of SEQ ID NO:12.

49. A pharmaceutical composition comprising an antigen binding protein as claimed in claim 48 and a further active agent, optionally an anti-inflammatory agent

50. A polynucleotide sequence encoding the antigen binding protein of claim 48.

51. A polynucleotide sequence as claimed in claim 50, wherein the polynucleotide comprises SEQ ID NO:141, 142, 143 or 144 and SEQ ID NO:11.
